SEQUENCE LISTING (49 sequences)

SEQ ID NO: 1 - DNA, 3726 bp, synthetic construct
  misc_feature 1..3726: note = cDNA of AtCPL3
  source 1..3726: mol_type = other DNA
atgcttgtagctcgatctggttgttctagaaccctaattcgaatgggcaacgatgaaaacttgatggtaatggtggatgttgaggaaggtgagattcctgattctgtcaacactgagattgaagttaagcacaagagtactactactacggccgatgttggcggagacgtcgatgttggggttgtggctggaggaagaggaggaggtggtggtggttctaatggcaactctagggtttggacaatggaggagttgatttctcagtatcctgcttaccgtccatatgcgaattcgggtttatctaatttagcttgggctcgagctgtgcagaacaaaccttttaatgaaggtttggtaatggattatgaaccaagagagagtgataagattgtgattgaagatagtgatgatgagaaagaagaaggtgagttggaggaaggtgaaattgatttggtcgacaatgcttctgatgacaatttggttgagaaggatactgaatctgttgtgttgataagtgctgataaagttgaagatgatcgaattctaaaggagagagatttggagaagaaagtgaaactaattcgtggtgttttggagagtacttcgttggtagaagcacagaccggatttgaaggagtttgttccagaatattgggggcgttagagtctttgcgagagctggtttcagataatgatgattttccgaagagggatactttagttcaattgtcatttgcttctcttcaaaccattaactatgtgttttgctcgatgaacaatatttccaaggagcgtaataaggagactatgtcaagattgctgactcttgtaaatgaccatttttcccaatttctctcattcaaccagaaaaatgagatagagaccatgaatcaggatttaagccgttctgctattgcagtttttgctggaaccagcagtgaagagaatgttaatcaaatgactcagccgagtaatggtgattcttttcttgccaaaaagctgacttcagaaagtacacatcgaggagccgcctacttaaggagtaggttgcctatgctgcctcttctagaccttcataaggatcatgatgcagacagccttccgtcgcccacaagggaaacaacaccaagtttacctgtaaatggtcgccatacaatggttagaccaggttttcccgttggtagagagagccaaacgactgagggtgccaaagtctattcatatgagagtgatgcccgtaaagcagtttctacctaccagcaaaaatttggtcttaattcagtgtttaagacagatgaccttccaagcccaaccccatcaggagaacctaatgatggcaatggagacgttggtggagaggtttccagttctgttgttaagagctcgaacccggggagtcacctaatttatgggcaagacgttcctctgccctccaattttaattctagaagcatgcctgttgcaaattctgtttctagcactgttccaccacatcatctgtcaattcatgctatttctgcaccaactgcctctgatcagacagtgaaaccttctgcaaagagtcgagatccaagactaaggcttgcgaaacctgatgctgccaatgtaaccatttattcgtactcgtctggcgacgctagaaatctttcaaaagtagagctttctgcagacttggtgaacccaagaaaacaaaaagccgctgatgaatttttaattgatgggcctgcatggaaaagacaaaagagtgatacggatgcaccaaaagcagctggaactggtggctggctagaggatacagaatcatcgggacttctaaaactggaatccaagcccaggctaattgagaacggtgtaacatctatgacatcaagtgttatgcccacgagtgctgtttctgtgagccaaaaagtacggacagcttcaactgatactgcatcattgcaatcccttttgaaggatattgcagtaaatcctacaatgctactgaatctcctgaaaatgggagaaagacaaaaggtacctgaaaaagctattcagaaacccatggatccaagaagagcagcacaactccctggctcttccgtacaaccaggggtatcaacaccgcttagtatacctgcatcaaatgctttagctgctaattctttaaactcaggagtacttcaggattcttcccaaaacgcccctgcagccgaatctggaagcattcgcatgaaacctcgtgatcctcgccgaatcctgcatggaagtactcttcaaagaacggactcttcaatggaaaagcagaccaaagtgaatgatccttccactctaggaaccttgactatgaagggtaaggcagaagatttggaaacacctccccagcttgatccacggcaaaatattagccagaatggtaccagcaaaatgaaaatttcgggtgaacttctcagtgggaagacaccagacttttcaacacaattcaccaaaaacctgaaaagtattgctgatatggttgtcgtatcacaacaacttggcaatcccccagcaagtatgcattcggtacagcttaagacggagagagatgttaaacataatccttcaaatcccaatgcccaggatgaggatgtgtcagtttcagcagcatcagtaacggctgcagctggtcccactcgttccatgaacagttggggagatgtggaacacctatttgaaggatatgatgacattcagagagtagctattcaaagagagagagttcgtaggttagaggaacagaataaaatgtttgcatctcaaaagctctctcttgtcttggatatagaccacacccttctcaattcagctaagtttaatgaggttgaatcccgccacgaggagatattaagaaagaaggaagaacaagatcgtgagaaaccatatagacatctctttcgtttcctgcacatgggaatgtggactaaactaagaccagggatttggaattttttggagaaggctagcaagctgtacgagttacatctttacactatgggaaacaaattgtatgctacagagatggccaagctgcttgatcccaaaggggttctatttaatggacgggtcatatcgaaaggagatgatggagatcctcttgatggagacgaacgagtacctaagagcaaagatttagaaggagttatgggtatggaatcgtctgtggtgatcatagatgactctgtccgagtgtggcctcaacacaaaatgaatttaatagctgttgaaagatatctttatttcccttgtagtagacggcaatttgggcttcttggtccttctcttcttgagttagatcgtgatgaggtacctgaggagggcacattggcatcttcattagcggttattgagaaaatacatcaaaatttcttctcgcacacttcattagatgaagttgatgtgagaaatattttagcttctgagcaacgaaagatattggctggttgtaggattgtatttagtcggataatcccagtgggtgaagccaaaccgcacttgcatcccctgtggcaaactgctgagcagtttggtgctgtctgcacaacccaggtggatgaacatgtcactcatgttgtcacaaattctcttggaaccgacaaggtaaattgggcactaaccagaggtagatttgttgttcatcctggctgggtggaagcatcagcttttctgtaccagagagcaaatgagaacttatatgccatcaacccgtaa

SEQ ID NO: 2 - DNA, 3774 bp, synthetic construct
  misc_feature 1..3774: note = cDNA of GmCPL3_1 (18g282300.2)
  source 1..3774: mol_type = other DNA
atgatttttggatcgttattggattgtgagaaattgggaaaattggagaagatggggaaggaggtagaggatgttgaagagggtgagatttcggatactgcttcggtggaagagatttcagcggaggatttcaataagcaagatgttaaggtgttaaataataacaataagcccaatggaagtgatgctagggtttgggctgttcatgatctttactcaaagtaccctaccatatgtcgtggctatgcatcgggtttgtataaccttgcttgggcacaggccgtgcagaacaagcctttgaatgatatttttgtgatggaagtggactctgatgccaatgctaacagtaacagcaacaactccaatcgactggcctctgttgctgtgaatcctaaggatgtggtggtggtggatgtggacaaagaggaaggggagttagaggagggtgagattgatgccgacgcggaacctgaaggagaagcagagagtgttgtggctgttcctgttgtttctgattcagagaagcttgatgatgtgaagagggatgtttctaattctgagcagcttggtgtgaggggtgttctggagggtgttaccgttgctaatgtggcggagtcgtttgctcaaacttgcagtaagctgcaaaatgctcttcctgaagtgctctctagacctgctgattctgagagggatgatctcgttcgcctgtcatttaacgctactgaagtggtttattctgtgttttgctccatggactctttaaaaaaggaacagaacaaggatagcatattaagattactttcttttgtgaaggatcaacaacaggctcaattattttctccagagcatataaaagagatccagggcatgatgactgcaattgattattttggtgctttagttaatagtgaggctattggcaaggagaaagaattgcagaccactgtgcagacccatgagataaagactcaggaaaatcaagctgtagaagctgctgaattgatttcttataataagcctttgcatagtgacataattggggcatcacatgctttaaaatttggacaaaatagtattaaaggtagaggggttctgctccctctgttagaccttcacaaggatcatgatgctgacagtttaccatcaccaacccgagaagcaccctcatgcttccctgtgaataaattactttccgttggagagcctatggttagttctgggtcagcagctgctaagccggagtctggaaagatggaacttgatagtgaaggttctaaatttcatctctatgaaactgatgctttgaaagctgtttccacatatcaacagaagtttggtcgaagttccctttttacaaatgataaatttccaagtccaactccttcaggtgactgtgaggatgagattgttgatacaaacgaggaggtctctagtgcttctactggtgattttttaacaagcactaagccaactcttttggatctgccacctgtttctgctacttccacagataggtccagcttgcatggattcattagttctagagttgatgcagcaggtcctgggtctttgccagtgaaaagctctgcaaagaatagagatcctaggcttcgtttcgttaattctgatgcaagtgccgtggataacccatctacattgatacataatatgcctaaagtggaatatgctggaacaacaatctcaaggaaacaaaaggctgctgaagaaccttctttggatgttactgtatcaaaaagacaaaaaagtccattggaaaatactgagcataatatgagcgaagtaagaactggaattggtggttggttggaagagcatactgggcctggagctcagtttatagagaggaatcatttaatggacaaatttggacctgaaccccaaaagactttgaatacagtcagtagttcttgtactggttctgataatttcaatgcaacaagcattagaaatgagcaggcaccaattacaagtagtaatgtgctagcttccttacctgctctattgaaaggtgcagctgtaaatccaaccatgctggttaacttacttagaatagcagaagcccagaagaaatctgctgattctgctacaaatatgctgttgcatccaacaagctcaaattcggctatgggaacagactcaactgcgagtattggttcatcaatggccactggtcttcttcaaagttctgttggaatgcttccagtttcatcacaatcaacttccatgacacaaacccttcaagatgattcaggaaagattcgcatgaaaccccgtgatccccggcgcattctccacactaataatactatccagaagagtgggaacttggggaatgagcaattcaaagccattgtatcccccgtgtctaacaaccagggaactggggacaatgtcaatgcccagaagcttgagggtagggtggatagtaaattagtgcctactcaaccaagtgcacaacctgatattgctcgacaattcgcccggaatctgaaaaacattgctgatattatgtctgtttcccaagaatcatcaactcacactcctgttgctcaaattttttcttcagcatctgttccccttacttcagatagaggggaacagaaatctgttgtgtcaaactctcagaacctggaggctggcatggtatcagctcatgaaacagctgcatcaggtacctgtcgatcccagaatacatggggagatgttgagcatctttttgaaggttatgatgagcagcagaaggctgctatacagagagagagagcaaggagaattgaagaacagaataaaatgtttgctgctcgaaaattgtgccttgtattggatctagatcacacactacttaattctgctaagtttgtggaagttgatcctgtgcatgatgagatattgagaaagaaagaagagcaggaccgtgagaaaccacacagacatctttttcgctttcctcatatgggaatgtggactaaactcagaccaggaatctggaacttcttggagaaggccagtaagctctatgagctgcatctttacactatgggaaacaagctatatgcaacagaaatggcaaaggtgcttgatccaaagggacttttgtttgctggaagagttatctctagaggtgatgatactgattcagttgatggtgaggagagggctcccaaaagcaaagatttggaaggcgttttgggtatggaatcatctgttgtaattatagatgattctgtgagggtctggcctcataacaaactgaacctgatagtggtggaaaggtatacatacttcccctgtagtagacgtcagtttggactgcctggcccttcccttctcgagattgatcatgatgagagacctgaagctggaactctggcatcctctctggcagttattgagaaaatacatcaaatcttttttgcttctcgatccttagaagaagtggatgtcagaaatatactagcatcagagcagagaaaaatcttagctggttgtcgcatagtatttagtagggtatttcctgttggtgaagcaaatcctcaccttcacccattatggcagacagctgaacagtttggtgctttttgcaccaatcagattgacgaacaggttactcacgttgttgcaaattcccctgggactgataaggtgaattgggctctaaacaacggaagatttgttgtccatcctggctgggtggaagcatcagcattgctttataggagggcgaatgagcaagattttgccattaaaccataa

SEQ ID NO: 3 - DNA, 3786 bp, synthetic construct
  misc_feature 1..3786: note = cDNA of GmCPL3_2 (08g257900.1)
  source 1..3786: mol_type = other DNA
atggtttttggatcgttattggattgtgaggtattgggaaaattggagaagatggggaaggaggctgaggatgtggaagagggtgagatttcggatactgcttcggtggaagagatttcagccgaggatttcaataagcaagatgttaagttgttaaataataacaataagcccaatggaagtgatgctagggtttgggctgttcatgatctttactcaaagtaccctactatatgtcgcggctatgcatcgggcttgtataatcttgcttgggcacaggccgtgcagaataagcctttgaatgatatttttgtgatggaagtggactctgatgccaatgctaacagtaaccgcaacagctcccatcgactggcctctgttgctgtgaatcctaaggatgtggtggtggtggatgtggacaaagaggaaggggagttggaggagggtgagatcgatgctgatgcggaacccgaaggagaagcagagagtgttgttgtggctgtttctgattctgagaagcttgatgatgtgaagatggatgtttctgattccgagcagcttggtgcgaggggtgttctggagggtgttaccgttgctaatgtggttgagtcgtttgctcaaacttgcagtaagctgcaaaatacccttcctgaagtgctctctagacctgctggttctgagaaggatgatctcgttcgcttgtcatttaatgctactgaagtggtttattctgtgttttgctctatggactcttcggagaaagaacagaacaaggatagcatattaagattgctttcttttgtgaaggatcaacaacaggctcaattattttctccagagcatgtaaaagagattcagggcatgatgactgcaattgattctgttggtgctttagtaaatagtgaggctattggcaaggagaaagaattgcagaccactgagataaagactcaagaaaattcagctgtagaagtgcagatccatgagataaagactcaggaaaatcaggctgtagaagctgctgaattgatttcttatagtaagcctttgcatagggacataactgggacatcacaggctttaaaatttggacaaaatagtattaaaggcagaggggttctgctccctctgttagaccttcacaaggatcatgatgcagacagtttaccatcaccaactcgagaagcaccttcctgcttccctgtgaataagttactttctgttggagagtctatggttagatctgggtcagcatctgctaagatggaacttgatagtgaaggttctaaatttcatctatatgaaactgatgctttgaaagctgtttccacatatcaacagaagtttggtcgaagttccctttttacaaatgataaatttccaagtccaactccttcaggtgactgtgaagatgaggttgttgatacaaacgaggaggtctctagtgcttctactggtgattttttaacaagcactaaaccaactcttttggatcagccacctgtttctgctacttccatggataggtccagcatgcatggattcattagttctagagtggatgcaacaggtcctgggtctttcccggtgaaaagctctgcaaagaatagagatcccaggcttcgtttcattaattctgatgcaagtgctgtggataacctgtctacattgataaataatatgtctaaagtggaatattctggaacaacaatctcaaggaaacaaaaggctgctgaagaaccttctttggatgttactgtatcaaaaagactaaaaagttctttggaaaatactgagcataatatgagcgaagtaagaactggaagtggtggttggttggaagagaatactgggcctggagctcagttgatagagaggaatcatttaatggacaaatttggacccgaagccaaaaagactttgaatacagtcagtagttcctgtactggttctgataatttcaatgcaacaagcattagaaatgagcaggcaccaattacagctagtaatgtgctagcttccttacctgctctattgaaagaagcatctgtaaatccaatcatgcttgttaacatacttagattagctgaagcccagaagaaatctgctgattctgctgcaattatgctgctgcatccaacaagctcaaatccggcaatgggaacagactcaacagcgagtattggttcatcaatggccactggtcttcttcaaagttctgttggaatgcttcctgtttcatcacaatcaacttccacggcacaaaccctacaagatgattcaggaaagattcgcatgaaaccccgtgatccacggcgcattctccacactaataatactatccagaagagtggggacttggggaatgagcaattcaaagccattgtatcccctgtgtctaacaaccagagaacaggggacaatgtcaatgccccgaagcttgagggtagggtggataataaattagtgcctactcaatcaagtgcacaacctgatattgctcgacaattcacccggaatctgaaaaacattgctgatattatgtctgtttcccaagaatcatcaactcacactcctgtttctcaaaatttttcttctgcatctgttccgcttacttcagatagaggggaacaaaaatccgttgtgtcaagctctcagaacctgcaggctgacatggcatctgctcacgaaacagctgcatcagttacctctcgatcccagagtacatggggagatgttgagcatctttttgaaggttatgacgagcagcagaaggctgctatacagagagagagggccaggaggattgaagaacagaataaaatgtttgctgctcggaaattgtgccttgtattggacctagaccacacgctacttaattctgccaagtttgtggaagttgatcctctgcacgatgagatattgagaaagaaagaagagcaggaccgtgagaaacctcacagacatctttttcgctttcctcatatgggaatgtggactaaactcagaccaggaatctggaacttcttggagaaggctagtaagctctatgagttgcatctttacactatgggaaacaagctatatgcgacagaaatggcaaaggtgcttgatccaaagggagttttgtttgctggaagagttatctctagaggtgatgatactgattcagttgatggtgaggagagggttcccaaaagcaaagatttggaaggcgttttgggtatggaatcatctgttgtaattatagatgattctgtgagggtctggcctcataacaaactgaacttgatagtggtggaaaggtatacatacttcccctgtagtagacgtcagtttggactgcctggcccttcccttcttgagattgatcatgatgagagacctgaagctggaactctggcatcctctttggcagttattgagaaaatacaccaaatcttttttgcctctcaatccttagaagaagtggatgtcagaaatatacttgcatcagaacagagaaaaatcttagctggttgtcgcatagtatttagcagggtatttcctgttggtgaagcaaatcctcaccttcatccattgtggcagacagctgaacaatttggtgctgtttgcaccaaccagattgatgaacaggttactcacgtagttgcaaattcccctgggactgataaggtgaattgggctcttaacaacggaagatttgttgtccatcctggctgggtggaagcatcagcattgctgtataggagggcgaatgagcaagattttgccattaaaccttaa

SEQ ID NO: 4 - DNA, 3759 bp, synthetic construct
  misc_feature 1..3759: note = cDNA of BvCPL3 (g12062.t1)
  source 1..3759: mol_type = other DNA
atgaaagtatcaggtggtgaagatggtgaaattccagaatcagacgccattgttgagatgaaacatcaatcaaagactacgacgacaacgacgacaacgataacatcggattcatcgaataaaaaagataataacagttattggatgagagatctgtacaagtattcatcgtacagaggatatggcgctgcgtcaggtttatacaatttagcatgggcgcaagcagtacagaataagccattgaatgaggtacttgttgagctggatgataagaaaaacaataaaaatgcaagtactgatgatactagtgttaataaggaacaaggtgaagtacaacaacattgtgttgaaagtaaagaggtttttgaggtggttgacagtgagaaagaagaaggagaattggaagaaggcgaaatcgattttgattctgatgatactggtaataatcataatagtaatggtaataaagttcaggatgattttggtggattggagatggatgatggtgagttggagaatcaagtgtcttctattcgtaaagttcttcataatgttactgttgctgaagctcataaatcatttgatattgtgtgtgctcgattgaggacttctttggagacgttgagagagttggttttgcatacatggtttccttccaaagacgcgctcattcaacaagcttttgctgcaattcagtgtgtatactctgtttacagttctatgagccctaccttaagagatcagaataaggataggatgtcaaggttgcttacttttgtcatggaccttagttctgttctttttacaccggaacagaggaaagaggtggagggtatgattacctctgttaaccccccgattgttccagtgaaacccaagtcgagggacaggcaggaagaattgcctgttacagagaaggctatcctcaccgattctaatacattgactgtgaatactggtgacaataagtcagacttattgaagaaagtgggacccgaattgtctgtttatcagtcggaaaaaaagaacactgacattttatcagaagcaatgagacattttccgtctagcttaaaggtcagaagcagtttcggccctctgcttgatctccataaagttcatgatgaagacagccttccatctcctaccagcaagacaatgccttccttgcctttctttgaaacggctcctcctcgagtagtgcatggtttgcagaaatctggtgtgcacccttatgaaactgaagcagtgaaagctgtgtcaagctaccaacaaagatttggtcggagtacattcttagcaacggacatgcttcccagtccaactccttctgaagatggcaatgaaggaggagctgatgactctaatgaagaggtttctagttctaatgcctatactaatgttgttagtaggacaacaaattcctctgtagtgccacaaccagttgtttcttctgctgcttatacaagtagttcaaccatgcaaggggtgatcagtggtactagtgctgagagttctagtgtcgggtcttctccttcactgcgagcctctgcaaagagccgagatcctaggcttcggcatcttaatcctaattttggctcgttagaccttagtttttgtccgtctcctatggttcccagttcagcatctaaattggagccgctaggggaaattatgaaatcaaagaagaccaaggcacttgaggggcgccttctggatggtcctactgccaagagaccaagaaatggcctggaaactgaagatatgtccatgaatgcaaatcaagtaaaaactctccagggaagtactcgaatggagacttctagttctagtattttgggaccacagtcttccagcagaggactcttgggcccagctattgatcctcggaaaccaggaagtggtaccgtttcctccgggattactacaaacaatcctagcatggcagtaaataaaacggcaaagccctcaatgaatgtcagtggttctccgtccttgcagtcactgttgaaagacattgcagggaatccaggagcatggatgaatattattaaggagcagaacaaatccagtgaacctttacaaagtgtgtcacattcaatgaactcaaattcaatattaggagcagcaccctctgctattgctgttccacctatatcatctggggtgggacagacaagtgcaggattgcttcaggtcccttctccgaaagtggtcacgagctcacaggatgattcggcaaaacttcgcatgaaaccccgtgaccctcgtcgtgccctgcatgccaatatggcccaaaggactggcagttctgtgccagaacagcctaaggtaaatggagtacacaacacaacaacacaaggactccaggagaatatcaatgctcaaagatatgttaatggaacgagtccaagtgctgcgtcatctcagaccccaatcctacctgatataactaagcaatttacaaagaacttgaaaaacattgctgacattatttcttccccacaaacgtcaagtatacagtctccgctagcagtaagttctttgtcggcgcaagctaattcggatacgacaagtattagttctggtggacaagcaagttgtagttctggtggacccgtgattactggtaatcagcgtactgtgtctgcattgaggcccgaagaagttgtctcgggccgtccacaatcacagaacaattggggagatgttgagcatctctttgacggatatgatgatcagcaaaaggcagctattcaacaagagagagctagaaggcttgatgagcagaacaaaatgtttgctgatcgcaagttgtgtcttgtgctggatctggatcacacgcttcttaattcagcaaagttttcagaggtagatcctgtgcatgatgaaattctgaggaagaaagaggaacaagatcgtgaaaagcctcgacgacatctctttcgttttccacatatggcaatgtggacaaaattacgacctggtatttggaatttcttagagaaggctagtaagctttttgaactgcatctctataccatggggaacaagttgtatgccactgaaatggcaaaagtgcttgatccaaagggaactctatttgctggacgagttatctcacggggtgatgatggggatcctattgatggtgatgagagggtaccgaagagcaaggatttagagggtgttatgggtatggaatcttctgttgtaataatagatgattctgctagagtttggccacataacaagctaaacttgatagtcgtggaaaggtatacttactttccttgtagtaggaaacaatttgggctcccaggaccttcacttcttgagattgatcatgatgagagaccagaagagggaactctggcatcttctttggcagtcattgagaaaatacaccaaaactttttctctcacaagtcccttgatgatgtggatgtgagaaatatattgggagctgagcagcggaagattcttgctggttgccgaatcttgtttagcagggttttccctgttggggaagctaatccccatttacacccactttggcagacggcagagcagtttggtgctgtgtgcaccaatcagctagatgaacaagttacccacgtagtggcaaattctttagggactgacaaggttaattgggctctgtcaactaaaagatttgttgtacaccctagctgggttgaagcttctgcattgctttacagaagggttaatgagcaagactttgccatcaaaacttga

SEQ ID NO: 5 - DNA, 3759 bp, synthetic construct
  misc_feature 1..3759: note = cDNA of TACPL3-A
  source 1..3759: mol_type = other DNA
atgcgcgtgactctcacgcccaaggacgaggattggctggtcgtgctgatgacgagggagcgcccgcggtcggcggtggtcgcgcctgggggggacgtcttcaccgccggtggcggcggggagacgtccgatggggactcctcagagtcgctggaagaaattagtgccgccgactttaaggagtcgtccagtggcaccgctgctccgtcggcgtcgtcgcagagatctagggtttggatggggtacaccatgtcccggagttatgcaccggcgttccacagctttgcttgggcgcaagctgtgcagaacaagcctctcgttccgcggcctgctgccgacgaggacgaggtagagcacctcgtcgacacctcggacgaggagaaggaagagggcgagattgaggagggggaggccgtacagtccacttcccctccaattaagcagcctgagaccatcgacttggactctgatgcgcaggacaagtcagagtcagtggacatggagcagacccgtttggccgttgaggcggctgatgagctggattttgaccagcgcgtggggagtatactggaggagcttgagaggctttccattgaggaagctgagaagtcatttgaggcttcgtgtgcccgcctgcggtcttgctttgagagccttaagccgctgttcccagagagcggtagcccgatgcctatgcttgatgctcttgtgcaacaggcttttgttggaatcgacaccatcaccactgtagctaattcatatgcgatgccgaagagggagcagaacaagaacatgctgttgaagctgctgtttcacataaagaacagatattcagacatgctggcactcaaccagcgagatgagctcgatagtcgtgtgagacagttagtttttgtagatggagaagacaatgccggttccaattgtagcaccaaaacagtgaatgtggttgttccatctggacaggttccatcagatagactgccagtcgagtcaggagcagcaaatccacctaggggctctagtttccccagctgggagataccggcgaataatagaattgttagccccttgttggaccttcatgcagattatgacgagaacagcttaccctcacccacccgagttagtgccccaccttttcctgtgccaaagcccattgggtttggagtatttccaatggcacctgacagatatttttcggcagaaagaattgatccttcaaaaaattttctttatccatgtgtgaatgatgcgctaaaggatgtttcctcgtaccgacagaagtatggcccgacgtctacctttgcaagtgatgatcttccaagcccaaccccatctgatgatggggataaatctggagacaaagaaggtgatatatttggtgaagtttcaagcttttcagcttctaataagtctgctccgccaagtgggaatctgatgcctgcttcccgacctagtgcagttatcagcagcaatgacagttttgcaggtggtcctccaggttatgctaaacaaattgaacagtctgtttcaggacccagccatgctcttaagccttcagctaaaagtagagatccaaggctcaggtttttgaaccgtgattctggtggtactgcagatgcaaatatacatgtaaatttggcagagccaaatgcttccaaagatgggaccttggggggtgttgtatcagataatagccggaagcacaaggcaactggccaacctctcatggatgaaaccgtgttaaaaagagctagggagagtactgggagtcccagagacattctggtaccacctggtagagatggaagtaacatcagctcctattcaggtgacagggttcaatcaaataagcatacagggcttgaaactaagacagccaggaatcctagtattaggaccagtagtcaacttattagcaatgtaagtagtatcccagacagtactggaactctccaagcctcccaacctaattcagttccacagaccagtgcagctcctattgtttcattgcctgcagtgttaaaggacattgctgtgaacccgactgtgctcatgcattggattcaaatggaacatcagaagaggtccgcatcagagcctcagcctgcttcaggtatcatctctagtggcatgatcaataatgtcactgctgggatggttataccacctggcaatgctctgaagaccgcagaagttgcacacattccttcttataggccacaagcaacatcgcaaacagcctctgtgaattcacaaaatgaccctggagtaatacgtatgaaggcgcgtgatccccgtcgtgtcctccacaataacacatcacagaagaacgatactctgaactctgatcaagccaaaagcaatggtatcgccctgccggccttccaggacagcaaagacaatttgattaaccgtcaacaactggcagagcaacttcagactactgtgttgccatctcaaccagtctcattatccagcattgctcgacagtccaccatgagcgcgagtaaggtcgatcctgtctctaattcacagttagctgcttcatcactcattgctcctcaagaaagtttagtcagcgtaaatagggcagatccaagagtagctgctggacagaatgattccaataatgctgcccctgctacaacacttggtaccaggccaccagctaaccagtggggtgatcttgatgatctccttaacggttatgatgaccagcagaaggctctcatacagaaggaaagggcaagacggatcatggaacagcacacgatgttttcatcgaggaaactttgtttagtgcttgatttggatcacactctcctcaattctgcgaagtttatagaagtggatcctattcatgaagagattttgcggaagaaagaggaacaagactgggaaaggtcagagcggcatctgttccgattccatcatatgcaaatgtggactaaactaagaccaggaatatggaattttctcgagaaggcgagcaagctttacgagttacatctgtacacgatggggaacaagctgtatgctactgagatggctaaggttcttgatcctagtggaaccctgtttgcagggagagtaatctcaaggggtggtgatggtatctcaagaggtggtgacggtgatacatttgacagcgatgaccgtgtaccaaaaagtaaagatcttgatggggtattggggatggaatctgcagtagtgatcatcgatgactctgtgagagtctggccccacaacaaaaacaatatgattgttgtagagagatacacctatttcccttgcagcagacggcaatttggccttcctggaccatcacttcttgaaattgatcgtgatgaaaggcctgaggatggcactcttgcttcttcgttggcggttattgggcgcattcatcaaaacttcttttctcatcccaacctcaatgatgctgatgtgcgcagcatactatcatctgagcagcggaggatccttgccggctgccgtattgtctttagccggattttccctgttggagaggctaacccccacttgcatcctctctggcagactgcagagcagttcggtgcagtgtgcacgaaccagattgacgatcgggttactcatgttgtcgccaactcactaggaaccgacaaggtgaattgggcactacaaacaggcagattcgtcgttcatccaggatgggtagaagcttcagcacttctataccggcgtgccaatgaacacgattttgcagtaaaataa

SEQ ID NO: 6 - DNA, 3732 bp, synthetic construct
  misc_feature 1..3732: note = cDNA of TACPL3-B
  source 1..3732: mol_type = other DNA
atgcgcgtgacgctcacgcccaaggacgaggattggctggtcgagctgatgacgagggagcgcccgcggtcggcggtggtcgcgcctgggggggacgtcttcaccgccagtggcggcggggagacgtctgatggggactcctcagagtcgctggaagaaattagtgccgccgactttaaggagtcgtccagtggcaccgccgctgcgtcggcgtcgtcgcagagatctagggtttggatgggatacaccatgtcccggagttatgcgccggcgttccacagctttgcttgggcgcaggctgtgcagaacaagcctctcgttccgcggcctgccgacgaggacgaagtagagcatctcgtggacgcctcggaggaggagaaggaagagggcgagattgaggagggggaggccgtacagtccacttcccctccaattaagcagcctgaggccatcgacttggactctgatgcgcaggacaagtcagagtcagtggccatggagcagacccctttggccgtcgaggcggctgatgagctggattttgaccagcgcgtggggagtatactggaggagcttgagaggctttccattgaggaagctgagaagtcatttgagggttcgtgtgtccgcctgcggtcttgttttgagagccttaagccgctgttcccagagagcggtagcccgatgcctatgcttgatgctcttgtgcaacaggcttttgttggaatcgacaccatcaccactgtagctaattcatatgcgatgccgaagagggagcagaacaagaacatgctgttgaagctgctgtttcacataaagaacagatattcagacatgctggcactcaaccagcgagatgagcttgatagtcgtgtgagacagttagtttttgtagatggagaagaccatgccggttccaattgtagctccaaaacagtgaatttggttgttccatctggacaggttccatcagatagactgccagtcgagtcaggagcagcaaatccacttgggggctctagtttccctagctgggagataccggcgaataatagaatggttagccccttgttggaccttcatgcagattatgacgagaacagcttaccctcacccacccgagatagtgcaccaccttttcctgtgccaaagcccattggatttggattatttccaatggcacctgacagatatttttcggcggaaagagttgatccttcgaaaaaagttctgtatccatgtgtgaatgatgcgctaaaggatgtttcctcgtaccgacagaagtatggccagacatctaccttcgcaagtgatgatcttccaagcccaaccccatctgatgatggtgataaatctggagacaaagaaggtgatatatttggtgaagtttcaagcttttcagcttctaatctgatacctgcctcccgacctagtgcagttatcagcagcaatgacagttttgcaggtggtcctccaggctatgctaaacaaattgaacagtctgtttcaggacccagccatgctcttaagccttcagctaaaagtagagatccaaggctcaggtttttgaaccgtgattctggtggtactgcagatgcaaatagacatgtaaatttggcagagccaaatgcttccaaagatgggaccttggggggtgttgtatcagataatagccggaagcacaaggcaactggccaacctctcacggatgaaaccgtgttaaaaagagctagggagagtactgggaatcccagagacatgcaggtaccacctggtagagatggaagtaacattagctcctattcaggtaacagggttcaatcaaatcagcataaagggcttgaaactaaggcagccgggaatcctagtattaggaccagcagtcaacttattagcaatgtaagtagtatcccagacagtactggaactctccaagcctcccaacctaattcagttccacagaccagtgcagctcctattgtttcattgcctgcagtgttaaaggacattgctgtgaacccgactgtgctcatgcattggattcaaatggaacatcagaagtggtcagcatcagagcctcagcctgcttcaggtatcatctctagtggcatgatcaataatgtcactgctgggatggttataccacctggcaatgctccgaagaccgcagaagttgcacacattccttcttataggccacaagcaacatcgcaaacagcctctgtgaattcacaaaatgaccctggagtaatacgtatgaaggcccgtgatccccgtcgtgtcctccacaataacacatcacagaagaacgatactccgaactctgatcaagccaaaagcaatggaatcgccctgccggccttccaggacagcaaagacaatttgattaaccgtgaacaactggcagagcaacttcagactactgtgttgccatctcaaccagtctcattatccagcattgctcgacagtccaccatgagcgcgagtaaggtggatcctgtctctaattcacagttagctgcttcatcgctcattgctcctcaagaaactttagtcagcgtaaatagggcagatccaagagtagctgctggacagaatgattccaatgatgctgcccctgctacaacacttggtaccaggccaccagctaaccagtggggtgatcttgatgatctcctcaacggttatgatgaccagcagaaggctctcatacagaaggaaagggcaagacggatcatggaacagcacacgatgttttcatcgaggaaactttgtttagtgcttgatttggatcacactctcctcaattctgctaagtttatagaagtggatcctattcatgaagagattttgcggaagaaagaggaacaagaccgggaaaggtcagagcggcatctgttccgattccatcatatgcaaatgtggactaaactaagaccaggaatatggaattttctcgagaaggcgagcaagctttatgagttacatctgtacacgatggggaacaagctgtatgctactgagatggctaaggttcttgatcctagtggaaccctgtttgcagggagagtcatctcaagaggtggtgatggcatctcaagaggtggtgatggtgatacatttgacagcgatgaccgtgtaccaaaaagtaaagatcttgatggggtattggggatggaatctgcagtagtgatcatcgacgactctgtgagagtctggccccacaacaaaaacaatatgattgttgtagagagatacacttatttcccctgcagcagacggcaatttggccttcctggaccatcacttcttgaaattgatcgtgatgaaaggccggaggatggcactcttgcttcttcgttggcggttattgggcgcattcatcaaaacttcttttcccatcccaacctcaatgatgctgatgtgcgcagcatactatcatctgagcagcggaggatccttgcgggctgccgtattgtctttagccggattttccctgttggagaggctaacccccacttgcatcctctctggcagactgcagagcagttcggtgcagtgtgcacgaaccagattgacgatcgggttactcatgttgttgccaactcactaggaaccgacaaggtgaattgggcactacaaacaggcagattcgtcgttcacccaggatgggtagaagcttcagcacttctataccggcgtgccaatgaacacgattttgcagtaaaataa

SEQ ID NO: 7 - DNA, 3759 bp, synthetic construct
  misc_feature 1..3759: note = cDNA of TACPL3-D
  source 1..3759: mol_type = other DNA
atgcgcgtgacgctcacgcccaaggacgaggattggctggtcgtgctgatgacgagggagcgcccgcggtcggcggtggtcgcgcctgggggggacgtcttcaccgccggtggcggcggggagacgtccgatggggactcctcagagtcgctggaagaaattagtgccgccgactttaaggagtcgtccagtggcaccgccgctgcgtcggcgtcggcgcagagatctagggtttggatgggatacaccatgtccaggagttatgcgccggcgttccacagctttgcttgggcgcaggctgtgcagaacaagcctctcgttccgcggcctgttgccgacgaggacgaggtagagcacctcgtcgacgcctcggacgaggagaaggaagagggcgagattgaggagggggaggccgtacagtccacttcccctccaattaagcagcctgagaccatcgacttggactctgatgcgcaggacaagtcagagtcagtggccatggagcagacccctttggccttcgaggcagctgatgagctggattttgaccagcgcgtggggagtatactggaggagcttgagaggctttccattgaggaagctgagaagtcatttgagggttcgtgtgcccgcctgcggtcttgctttgagagccttaagccgctgttcccagagagcggtagcccgatgcctatgcttgatgctcttgtgcaacaggcttttgttggaatcgacaccatcaccactgtagctaattcatatgcgatgccgaagagggagcagaacaagaacatgctcttgaagctgttgtttcacataaagaacagatattcagacatgctggcactcagccagcgagatgagctcgatagtcgtgtgagacagttagtttttgtagatggagaagacaatgccggttccaattgtagcaccaaaacagtgaatgtggttgttcaatctggacaggttccatcagatagactgccagtcgagtcaggagcagcaaatccacttaggggctctagtttccctagctgggagataccggcgaataatagaatggttagccccttgttggaccttcatgcagattatgacgagaacagcttaccctcacccacccgagatagtgcaccaccttttcctgtgccaaagcccattggatttggagtatttccaatggcacctgacagatattttttggcggaaagagttgatccttcaaaaaaagttctgtatacatgtgtgaatgatgcgctaaaggatgtttcctcgtaccgacagaagtatggccagacatctacctttgcaagtgatgatcttccaagcccaaccccatctgatgatggggataaatctggagacaaagaaggtgatatatttggtgaagtttcaagcttttcagcttctaataagtctgctccgccaagtgggaatctgatacctgcctcccgacctagtgcagttatcagcagcaatgacagttttgcaggtggtcctccaggctatgctaaacaaattgaacagtctgtttcaggacccagccatgctcttaagccttcagctaaaagtagagatccaaggctcaggtttttgaaccgtgattctggtggtactgcagatgcaaatagacatgtaaattttgcagagccaaatgcttccaaagatgggaccttggggggtgttgtatcagataatagccggaagcacaaggcaactggccaacctctcacgggtgaaaccgtgttaaaaagagctagggagagtactgggaatcccagagacatgcaggtaccacctagtagagatggaagtaacattagctcctattcaggtgacagggttcaatcaaatcagcataaagggcttgaaactaaggcagccgggaatcctagtattaggaccagtagtcaacttattagcaacgtaagtagtatcccagacagtactggaactctccaagcctcccagcctaattcagttccacagaccagtgcagctcctattgtttcattgcccgcagtgttaaaggacatcgctgtgaacccgactgtgctcatgcattggattcaaatggaacatcagaagaggtcagcatcagagcctcagcctgcttcaggtattatctctagtggcatgatcaataatgtcactgctgggatggttataccacctggcaatgctctgaagaccgcagaagttgcacacattccttcttataggccacaagcaacatcgcaaacagcctctgtgaattcacaaaatgaccctggagtaatacgtatgaaggcccgtgatccccgtcgtgtcctccacaataacacatcacagaagaacgatactccgaactctgatcaagccaaaagcaatggaatcaccctgccggccttccaggacagcaaagacaatttgattaaccgtgaacaactggcagagcaacttcagactactgtgttgccatctcaaccagtctcattatccagcattgctggacagtccaccatgagcgcgagtaaggtggatcctgtctctaattcacagttagctgcttcatcactcattgctcctcaagaaactttagtcagcgtaaatagggcagatccaagagtagctgctggacagaatgattccaatgatgctgcccctgctacaacacttggtaccaggccaccagctaaccagtggggtgatcttgatgatctcctcaacggttatgatgaccagcagaaggctctcatacagaaggaaagggcaagacggatcatggaacagcacacgatgttttcatcgaggaaactttgtttagtgcttgatttggatcacactctcctcaattctgcgaagtttatagaagtggatcctattcatgaagagattttgcggaagaaagaggaacaagaccgggaaaggtcagagcggcatctgttccgattccatcatatgcaaatgtggactaaactaagaccaggaatatggaattttctcgagaaggcgagcaagctttacgagttacatctgtacacgatggggaacaagctgtatgctactgagatggctaaggttcttgatcctagtggaaccctgtttgcagggagagtcatctcaagaggtggtgatggcatctcaagaggtggtgatggtgatacatttgacagcgatgaccgtgtaccaaaaagtaaagatcttgatggggttttggggatggaatctgcagtagtgatcatcgacgattctgtgagagtctggccccacaacaaaaacaatatgattgttgtagagagatacacctatttcccctgcagcagacggcaatttggccttcctggaccatcacttcttgaaattgatcgtgatgaaaggccggaggatggcactcttgcttcttcgttggcggttattgggcgcattcatcaaaacttcttttctcatcccaacctcaatgatgctgatgtgcgcagcatactatcatctgagcagcggaggatccttgccggctgccgtattgtctttagccggattttccctgttggagaggctaacccccacttgcatcctctctggcagactgcagagcagttcggtgcagtgtgcacgaaccagattgacgatcaggttactcatgtcgttgccaactcactaggaaccgacaaggtgaattgggcactacaaacaggcagattcgtcgttcacccaggatgggtagaagcttcagcacttctataccggcgtgccaatgaacacgattttgcagtaaaataa

SEQ ID NO: 8 - DNA, 3594 bp, synthetic construct
  misc_feature 1..3594: note = cDNA of SbCPL3 (Sb05g019010.1)
  source 1..3594: mol_type = other DNA
atggcgcgggagcggccgcggtccacggtggttgcggcgggcggagatttggtcactgctcccggcggcggcgaggggtcggatggggactcggcggggtcgatagaggagatttcagctgacgacttcaggaaggactcgtcgtcggcgttgggcggccccgccgccgcggcggctgcggggcagagatctaggtcttgggtgggtccgcccgccgtgggttacatggcgcggaactttgggcacgccttcaacagcttcgcgtggtcgcaggccgtgcggaacaagccgctggggttacagccgccgcctgcctcggacgaggacgaggtggagcatgccgtggacgcgtccgacggggagaaggaggagggcgagattgaggagggggaggctgtggaggccgaggcatcgcctgcccgcgcacagcctgagaccatcgacttggacgcggacgcggacgcgctggagaagtcagagtcgctggctggtgctgtgcctgcctctgctgctgaggaagaggaggtgaaccttgatcagcgtgtcggcagtatactggaggagcttgagatggtctccattgaggaagccgagaagtcttttgagggtgcatgtggacgactgcatacctgctttgagaacctgaagccgctgttccaggaactggagaacgggagcccgatggctatacttgaacccctcatgcagcaggcgttcattggaattgacacactcaccactgtggcgatttcgtataacttgccgaggagtgagcagaataagacaacgcttttgaagtcgttgttccacataaagaacagatattcagacatgctgactcccgagcagcgagacgagttggacagccgcgtgaggaagttagtttttggagaaaaagacaatgtcagtgacccaagtacaagtagtggcaccaatgcgataaatgttttggctccatctgggcaggtctcatcctcaggaggactgccatttgaatcaggtgcagcaaatccatttagtagcttgccgaggttggaagtacctgccaaaaggattagtcccttgttggatcttcatgcagattatgatgaaaacagcttaccatcgccaaccagggataatgcaccgccttttcctgtgccaaagcctattggttttggagcatttccaatggtgcctgagaaattatctttcccagaaagagttgagcctgcaaagaattcgttatatccatccttaaatgatcctctgaaggctgtctcctcatatcagcagaagtatgggcagaaatctgtatttccaagtgatgatctgccaagtccaactccatctggtgatgagggtaaatctgcagataaaggtggtgacatatttagtgaggtttccagcttccctgttccaaagagtattgcattgccaagtacaagccagatgcctgcttctcaacctagcacagtcagcagcagtggtattagttatgcatctggtccacctggttttgctaaacaaattgagcagccggttgcaggtccaaatcacgcaataaaggcagcatcgaaaagtagagatccaaggctcaggtttttgaaccgtgattctgctggtgctacagatgtgaatcggcgcgcaaatttttcagaactgaaggatgggaacttgggtggagcgtcagttggtaaccgtaaacacaaagcaattgatgatcctcaggtggatgaaaatgtgttaaaaagatttaggggcggaactgcaaatccgagagacttgcaacctacagggaatcctaatcagcttatgaacattagggctcctacaaatagtagcggtatcaatatgaaaactttgcagccccctcaaactactgctccacatgtcagtgcagctcctgctgtcccagtgccttctatgttgttaaaggacattgctgtgaacccaacattgcttatgcatttgatccaaatggaacatcaaaagaagtcagcatcagaaactcagggtggcatgtctagtgggatgagcaacaatggaattgcaggcatggttttcacacctggcaatgctccaaagaccacagaagctgcacaagtcccctctgttaggccacaggttccagcgcagacaccttctttgaactcacaaaatgatggtggaatccttcgcatgaagccccgtgatccacgacgcatcctacacaataacgtagcacagaaatctgatgcaatggtcttggagcaagtgaaaactaatggaattacccagccagactctcagggcaccaaggaccagactagttcaatgccttctcaaccaactttgccatctagcgttgcaaggccattcacaaacacaaaacatgttgatcctgtctctaattcacagttagctgctacagctataatggctcctacacaacaagctttgggcagcataaataaggtggatccaagactagcagttgaacagaatgggcaaaatgctgatgcaacaacaactgatgcttctgcaacagaacttgaagctacacagcctgttagcccatggggtaatcttgatcatctccttgatggatatgatgataagcaaaaagctctgatacagaaggaaagggcaagacgaataacggaacagcacaaaatgttctcagcgcgaaagctatgcctggtgcttgatttggatcacacccttcttaattctgcaaagtttatagaagtggaacccattcatgaagagatgttacggaagaaagaggagcaagacaggactttgccagaacgtcatctctaccgttttcatcatatgaatatgtggacgaagttgaggccaggaatatggaactttcttgagaaggctagtaacctattcgagttgcatttgtacactatgggaaacaaactgtatgctactgagatggccaaggttcttgatcctactggaaccttgtttgctggacgagtcatatcaagaggtgatgatggcgatccctttgacagtgatgagcgagtgccgaaaagtaaagatttggatggggtactgggtatggaatctgcagtcgtgatcatagatgattctgtaagagtctggcctcataacaggcacaatttgatagttgtagaaagatacacctatttcccctgcagcagacgtcaatttggccttcctggaccatcacttttagagattgacagagacgagaggcctgaggatggtacccttgcttcttcacttgcggtcattgagagaatccaccacaatttcttttcacatcctaacctcaatgaggctgatgtgcggagcatactagcatctgagcagcgaaggatccttgctggttgtcgtattgtctttagccgggttttcccagttggcgacgccagcccccacttgcatcctctgtggcagactgcagagcagtttggtgcggtctgcacaaacctggttgatgatcgggttactcatgttgttgccaactcccctgggacagacaaggtgaattgggcattatccaaaggcaaatttgtggtgcatccaggatgggtagaagcttcagccctgttgtaccggcgcgccaacgaacatgactttgcagtcaaataa

SEQ ID NO: 9 - DNA, 3705 bp, synthetic construct
  misc_feature 1..3705: note = cDNA of ZmCPL3
  source 1..3705: mol_type = other DNA
atgcgcgtgacggtcacgcccaaggacgaggaaaggctggttgacttgatggcgcgcgagcagccgcggtcggcggtggttgcggcgggcggagatttggtcactgctgccgggggcgggggcgaggggtcggatagggactcttcggggtccatagaggagattacggctgacgacttcaagaaggactcgtcgtcggcgttgggcggcgcggcggcggcggcggggccgagatctaggtcttgggtggccccgcccgccgtgggttacatggcccggaactttcgctatgccttcaatagctttgcgtggtcgcaggccgtgcggaataagccgctggggctacaaccgcctgccccagacgacgacgaggtggagcacgccgtggacgtctccgacggagagaaggaagagggcgagattgaggagggggaggctgtggaggccttggcctcgcctgctcctgcgcagcctgagaccatcgatttggactccgacgccccggagaagtcagagtcggtggctatcgatggaagtgccagtgttgtgcctgtcccagctgctgaggaagaggaggtgaaccttgatcagcgtgtggggagtatactggaggagctcgagatggtctccattgaggaagctgagaagtcgttcgagggtgcatgcgcacgactgcatacctgcttcgagaacctgaagcctctgttccaggaactggagaacgggagcccgatggctatccttgaacccctcatgcagcaggcgtttattggaattgacacactcaccactgtggcgaatttgtataacttgccgaggagggagcagaataagacaacacttttgaagttgttgttccacataaaaaacagatattcggacatgctgacgcctgagcagcgagaagagatggacagccgcgtgaggaagttagtttttggagagaaagacaatgtcagtgacccaagtaccagttgtggcaccagtgcgataaatgtttcagctccatctgggcaggtttcaaacacaggaggactgccatttgaatcaggtgcagcaaatctatttagtagcttgccaaggttggaagtacctgccaaaaggaatagtcccttgttgaatcttcatgcagattatgatgagaacagcttaccctcgccaacccgggacaatgcaccgccttttcctgcgctaaagcctattggttttggagcatttccaatggtacctgagaaactatctttcctagacagagttgagcctacaaagaattcgttatatccacccttaaatgatcctctgaaggctgtctcttcctatcagcagaagtatgggcagaaatctgtatatccaagtgatgatctaccaagtccaactccatctggtgatgagggtaaacctgcagataaaggtggtgatatatttagtgatgtttccagcttccctgttccaaagagtattgtattaccaagtacaagtcagatgcctgcttctcaacctagcacagtcagcagcagcagtattagttatgcttcaagtacaagtcagatggctgcttctcaacctatcacagtcagcagtagcggtattagttatgcgtctggtccacctggttttgctaaacaaattgagcagtcgactgcaggtccgaatcatgcaataaaggcagcatccaaaagtagagatccgaggctcagatttttgaaccgtgattctgctggtgctacagatgtaaattggcgtgcaaatttttcagaactgaaggatgggaacttgggtggagtttcagttggtaaccgtaaacagaaagcagttgatgatcctcaggtggatgacaatgcgttaaaaagatttaggggcggaattgcgaatcagagagacatgcagcctacggggaaccccaatcagcttatgaacatcagggctcctacacatagtagcagtatcaatatgaaaactttgcaacctcctcaaactactgctccacatgtcagtgcggctcctgctgtcccattgcctcctatgttgttaaaggacattgctgtgaacccagcattgcttatgcatttgatccaaatggaacatcaaaagaagtcagcatcagaaagtcagggtggcatgtctagtgggatgaccaacaatggaattgcaggcatggttttcacacctggcaatgctccgaagatcacagaagctgcacaagtcccatctgttaggccacaggttccagtgcagacacctcctttgaactcacaaaatgatggtggaatcgttcgcatgaagccccgtgatccacggcgcatcctacacaataacatagcacagaaatctgatgcaatgagcttggagcaagtgaaaaataatggaactacacagccagactctcagggcaccaaggaccagactactccagtgccttctcaaccagccttgccgtctagcattgcaaggccattcagcagcgcaaaacatgttgatcctgtctctaattcacagttagctgctacagctattatggctccgacacaagctttgagtagcgtaaataaggtggatccaagactagcagttgaacagaatggacaaaatgctgatgcaacaacaaatggtgcttctgcaacaacacttgaagctacacagcctgttagtccatggggtgatgttgatcatctccttgatggatatgatgaccagcaaaaggctctgatacagaaggaaagggcaagacgaataacagaacaacacaaaatgttctcagcacggaagctatgcttggtgcttgatttggatcacacccttcttaattctgcaaagtttatagaagtggaaccaattcatgaagagatgttacggaagaaagaggagcaagacaggactttgcctgaacgtcatctctaccgttttcatcatatgaatatgtggacgaagctgaggccaggaatatggaactttcttcagaaggctagtaacctgtttgagttgcatttatacactatgggaaacaaactgtatgctactgagatggccaaggttcttgatcctactgggaccttgttcgctggacgagtcatatcaagaggtgatgatggtgatccttttgacagtgatgaacgagtgccaaaaagtaaagatctggatggggtactgggtatggaatctgcagttgtgatcatagatgattctgtaagagtctggcctcataacaggcacaatttgatagttgtagagagatacacctatttcccctgcagcaggcgtcaatttggccttcctggaccatcacttctagagattgacagagatgagaggcctgaggatggtacccttgcttcttcacttgcggtcattgagagaatccaccacaattttttttcacatcctaacctcaatgaggctgacgtgcggagcatactagcatctgagcagcgaaggatacttactggttgccgtattgtctttagccgggttttcccggttggagacgccagccctcacttgcatcctctgtggcagactgcagaacagtttggtgcagtctgcacaaacctggttgatgatcgggtcactcatattgttgcaaactcccctgggacagacaaggtgaattgggcattatccaaaggcaaattcgtagtgcatccaggggtagaagcttcagctctgttgtaccggcgcgccaacgaacatgactttgcagtcaaataa

SEQ ID NO: 10 - DNA, 3657 bp, synthetic construct
  misc_feature 1..3657: note = cDNA of StCPL3 (PGSC0003DMT400052897)
  source 1..3657: mol_type = other DNA
atggaagaatgtaatcgagttgaagatgtagaggagggtgagatctcagattcggcttcagttgaagaaataagtgaggatgcgttcaataggcaagacccacctactactactaagattaagattgcgagtaatgaaaatcaaaatcaaaattcgacgacgacgactagggtttggacgatgagagatgcatataaatatccaatttctagggattatgctagaggtttgtataacctggcttgggctcaggctgtgcagaataagcctttagacgaactattcgtcatgactagtgacaattccaaccagtgtgctaatgctaatgctaatgttgaatccaaggtcatcattgatgtggatgtggatgacgatgctaaagaggaaggagagttggaggaaggcgagatcgacttggatgccgctgacctagttctaaattttggaaaggaggccaatttcgttagggaacaacttcagagtgttactctagacgaaacccacaaatccttttctatggtttgttccaaattgcaaacttcattactggccttgggggaactagctctttctcaagacaagaatgacattcttattcaactctttatgactgcactccgaaccattaattctgttttttactccatgaaccaggatcagaagcaacaaaacacagatattctatctaggttgctttttcatgcaaagacacaattacctgctcttttgtcttctgagcagttaaaagaggtggatgctgtgattctctctattaaccagtcagctgttttctcaaatactcaagacaatgacaaggtcaatgggatcaaagttgttgaactgttagataagaaggtttctcataaatcctctgaaaatgcaaatcaggattttactgctgtaaacaagtatgatttaggtgctgtatctatcaaatcttcaggcctgaaggaacagagtgtgtcatttgaatctgtaaaaccaggattagccaattctaaagctaaagggttatccattcctttgctagacctccataaggaccatgatgaagatactcttccgtcgcctacacgagagattggaccacagttccctgttgcaaaagctacacaggctcatggaatggtgaaactggacttgcctatttttgcgggttctcttgagaaagggaattctttattgcatccttatgaaactgatgcccttaaagctgtttcctcttatcaacaaaaatttggtcgaagttccctttttgttagtgaaaaccttccaagtccaactccgtctgaagagggtgatagtggcaaaggggacattggtggggaggtcaccagtcttgatgttgtacataatgctagccatctgaatgaatctagcatggggcaaccaatattgtcttctgttccccagaccaatattcttgatggacaaggactgggaactgctcggactgcagatcctctgagttttctgccaaatccttctttgcgatcttctacagcaaaaagtagagatcccagactcagactggcaaccagcgatgcagttgctcagaacacgaataaaaatatcttgcctatcccagacattgatttgaaattggaggcttctttagagatgattggttcaaaaaagcagaagacagtagacctaccagtctttggtgctccattgccgaaaagacaaagaagtgaacagactgattcaatcattgtgagtgatgtgcgtccttcgactggaaatggtggttggttagaggatagagggactgctgggttgccaattacgagtagtaactgtgctacagatagcagtgacaatgatattaggaaattggagcaagtaacagctactatcgctactatacctagtgtcatagttaatgctgctgagaactttccagtgactggcattagcacgtcaacaactttgcattctttgttaaaagatatagctataaatccatcgatatggatgaatataatcaagatggaacagcagaaatctgctgatgcttctagaactactacagcacaagcttcaagttctaaatctattcttggagcagttccatcaacggatgcaatagctcccagatcttctgctattggccagagatcagtaggaatacttcagactcctacacacacagcatcagcggatgaagtggctatagtccgcatgaaacctcgtgaccctcggcgtgttcttcataatactgcagttctaaagggtggtaatgttggatcagatcaatgtaaaactggtgtagcaggcacacacgcaacgataagcaatcttggtttccaaagtcaagaggaccagttggataggaagtcagctgtgactctttcgactacaccaccagacattgctcgccaattcaccaaaaatttgaaaaatattgctgacatgatctctgtttcaccatccacatcactgtctgctgcttctcaaactcagacacaatgcctacaatctcatcagagtagatcggaaggtaaggaagcagtttctgaaccaagtgaacgggtgaatgatgctggcttagcttctgaaaaaggttctcctggttcattgcaaccacagatctcttggggagatgttgagcatctatttgaggggtacagcgaccaacagagagctgatatccagagagaaagggctaggaggcttgaggaacagaaaaaaatgttttctgttcggaagctctgtctcgtcttggacttggaccatactcttctgaattcagcaaagtttgttgaaatcgacccagttcatgaagagatattgaggaagaaagaggaacaagaccgtgaaaagccgtgtaggcacctattccggtttccacacatgggaatgtggaccaagttacggcctgggatttggaatttcttggaaaaggctagcaatctttttgagctgcatctctataccatgggtaacaagctatatgccacggagatggccaaattgctagatccaaaaggggatctgtttgctggacgagtgatctccaggggtgacgatggagatccatttgatggggatgaaagggttcctaagagtaaggacttggagggggttttgggcatggagtcagctgttgtgattattgatgattctgtgagagtctggccacataacaagctaaatttgattgttgtagagaggtatatttactttccttgcagtagacggcaatttggtctccctggtccttctcttcttgagattgatcatgatgaaagaccagaagatgggacattggcctcttgtttgggggttattcaaagaatacatcagaattttttcgcacatcggtccatagatgaagctgatgttaggaacattttagcgacagaacaaaaaaagattctggcaggttgccgtattgtcttcagcagagtgttccctgttggtgaagccaatcctcatttgcatcctctgtggcagacagctgaacagtttggtgctgtctgcactagtcaaattgatgatcaggttacccatgtggttgcaaattctcttgggacggataaggttaattgggcactttccaccggacgatttgttgttcatcctggctgggtggaggcatcagctttactttatcggagggcaaatgagcatgattttgctattaaatcttaa

SEQ ID NO: 11 - protein, 1257 aa, Glycine max
MIFGSLLDCEKLGKLEKMGKEVEDVEEGEISDTASVEEISAEDFNKQDVKVLNNNNKPNGSDARVWAVHDLYSKYPTICRGYASGLYNLAWAQAVQNKPLNDIFVMEVDSDANANSNSNNSNRLASVAVNPKDVVVVDVDKEEGELEEGEIDADAEPEGEAESVVAVPVVSDSEKLDDVKRDVSNSEQLGVRGVLEGVTVANVAESFAQTCSKLQNALPEVLSRPADSERDDLVRLSFNATEVVYSVFCSMDSLKKEQNKDSILRLLSFVKDQQQAQLFSPEHIKEIQGMMTAIDYFGALVNSEAIGKEKELQTTVQTHEIKTQENQAVEAAELISYNKPLHSDIIGASHALKFGQNSIKGRGVLLPLLDLHKDHDADSLPSPTREAPSCFPVNKLLSVGEPMVSSGSAAAKPESGKMELDSEGSKFHLYETDALKAVSTYQQKFGRSSLFTNDKFPSPTPSGDCEDEIVDTNEEVSSASTGDFLTSTKPTLLDLPPVSATSTDRSSLHGFISSRVDAAGPGSLPVKSSAKNRDPRLRFVNSDASAVDNPSTLIHNMPKVEYAGTTISRKQKAAEEPSLDVTVSKRQKSPLENTEHNMSEVRTGIGGWLEEHTGPGAQFIERNHLMDKFGPEPQKTLNTVSSSCTGSDNFNATSIRNEQAPITSSNVLASLPALLKGAAVNPTMLVNLLRIAEAQKKSADSATNMLLHPTSSNSAMGTDSTASIGSSMATGLLQSSVGMLPVSSQSTSMTQTLQDDSGKIRMKPRDPRRILHTNNTIQKSGNLGNEQFKAIVSPVSNNQGTGDNVNAQKLEGRVDSKLVPTQPSAQPDIARQFARNLKNIADIMSVSQESSTHTPVAQIFSSASVPLTSDRGEQKSVVSNSQNLEAGMVSAHETAASGTCRSQNTWGDVEHLFEGYDEQQKAAIQRERARRIEEQNKMFAARKLCLVLDLDHTLLNSAKFVEVDPVHDEILRKKEEQDREKPHRHLFRFPHMGMWTKLRPGIWNFLEKASKLYELHLYTMGNKLYATEMAKVLDPKGLLFAGRVISRGDDTDSVDGEERAPKSKDLEGVLGMESSVVIIDDSVRVWPHNKLNLIVVERYTYFPCSRRQFGLPGPSLLEIDHDERPEAGTLASSLAVIEKIHQIFFASRSLEEVDVRNILASEQRKILAGCRIVFSRVFPVGEANPHLHPLWQTAEQFGAFCTNQIDEQVTHVVANSPGTDKVNWALNNGRFVVHPGWVEASALLYRRANEQDFAIKP

SEQ ID NO: 12 - protein, 1261 aa, Glycine max
MVFGSLLDCEVLGKLEKMGKEAEDVEEGEISDTASVEEISAEDFNKQDVKLLNNNNKPNGSDARVWAVHDLYSKYPTICRGYASGLYNLAWAQAVQNKPLNDIFVMEVDSDANANSNRNSSHRLASVAVNPKDVVVVDVDKEEGELEEGEIDADAEPEGEAESVVVAVSDSEKLDDVKMDVSDSEQLGARGVLEGVTVANVVESFAQTCSKLQNTLPEVLSRPAGSEKDDLVRLSFNATEVVYSVFCSMDSSEKEQNKDSILRLLSFVKDQQQAQLFSPEHVKEIQGMMTAIDSVGALVNSEAIGKEKELQTTEIKTQENSAVEVQIHEIKTQENQAVEAAELISYSKPLHRDITGTSQALKFGQNSIKGRGVLLPLLDLHKDHDADSLPSPTREAPSCFPVNKLLSVGESMVRSGSASAKMELDSEGSKFHLYETDALKAVSTYQQKFGRSSLFTNDKFPSPTPSGDCEDEVVDTNEEVSSASTGDFLTSTKPTLLDQPPVSATSMDRSSMHGFISSRVDATGPGSFPVKSSAKNRDPRLRFINSDASAVDNLSTLINNMSKVEYSGTTISRKQKAAEEPSLDVTVSKRLKSSLENTEHNMSEVRTGSGGWLEENTGPGAQLIERNHLMDKFGPEAKKTLNTVSSSCTGSDNFNATSIRNEQAPITASNVLASLPALLKEASVNPIMLVNILRLAEAQKKSADSAAIMLLHPTSSNPAMGTDSTASIGSSMATGLLQSSVGMLPVSSQSTSTAQTLQDDSGKIRMKPRDPRRILHTNNTIQKSGDLGNEQFKAIVSPVSNNQRTGDNVNAPKLEGRVDNKLVPTQSSAQPDIARQFTRNLKNIADIMSVSQESSTHTPVSQNFSSASVPLTSDRGEQKSVVSSSQNLQADMASAHETAASVTSRSQSTWGDVEHLFEGYDEQQKAAIQRERARRIEEQNKMFAARKLCLVLDLDHTLLNSAKFVEVDPLHDEILRKKEEQDREKPHRHLFRFPHMGMWTKLRPGIWNFLEKASKLYELHLYTMGNKLYATEMAKVLDPKGVLFAGRVISRGDDTDSVDGEERVPKSKDLEGVLGMESSVVIIDDSVRVWPHNKLNLIVVERYTYFPCSRRQFGLPGPSLLEIDHDERPEAGTLASSLAVIEKIHQIFFASQSLEEVDVRNILASEQRKILAGCRIVFSRVFPVGEANPHLHPLWQTAEQFGAVCTNQIDEQVTHVVANSPGTDKVNWALNNGRFVVHPGWVEASALLYRRANEQDFAIKP

SEQ ID NO: 13 - protein, 1218 aa, Solanum tuberosum
MEECNRVEDVEEGEISDSASVEEISEDAFNRQDPPTTTKIKIASNENQNQNSTTTTRVWTMRDAYKYPISRDYARGLYNLAWAQAVQNKPLDELFVMTSDNSNQCANANANVESKVIIDVDVDDDAKEEGELEEGEIDLDAADLVLNFGKEANFVREQLQSVTLDETHKSFSMVCSKLQTSLLALGELALSQDKNDILIQLFMTALRTINSVFYSMNQDQKQQNTDILSRLLFHAKTQLPALLSSEQLKEVDAVILSINQSAVFSNTQDNDKVNGIKVVELLDKKVSHKSSENANQDFTAVNKYDLGAVSIKSSGLKEQSVSFESVKPGLANSKAKGLSIPLLDLHKDHDEDTLPSPTREIGPQFPVAKATQAHGMVKLDLPIFAGSLEKGNSLLHPYETDALKAVSSYQQKFGRSSLFVSENLPSPTPSEEGDSGKGDIGGEVTSLDVVHNASHLNESSMGQPILSSVPQTNILDGQGLGTARTADPLSFLPNPSLRSSTAKSRDPRLRLATSDAVAQNTNKNILPIPDIDLKLEASLEMIGSKKQKTVDLPVFGAPLPKRQRSEQTDSIIVSDVRPSTGNGGWLEDRGTAGLPITSSNCATDSSDNDIRKLEQVTATIATIPSVIVNAAENFPVTGISTSTTLHSLLKDIAINPSIWMNIIKMEQQKSADASRTTTAQASSSKSILGAVPSTDAIAPRSSAIGQRSVGILQTPTHTASADEVAIVRMKPRDPRRVLHNTAVLKGGNVGSDQCKTGVAGTHATISNLGFQSQEDQLDRKSAVTLSTTPPDIARQFTKNLKNIADMISVSPSTSLSAASQTQTQCLQSHQSRSEGKEAVSEPSERVNDAGLASEKGSPGSLQPQISWGDVEHLFEGYSDQQRADIQRERARRLEEQKKMFSVRKLCLVLDLDHTLLNSAKFVEIDPVHEEILRKKEEQDREKPCRHLFRFPHMGMWTKLRPGIWNFLEKASNLFELHLYTMGNKLYATEMAKLLDPKGDLFAGRVISRGDDGDPFDGDERVPKSKDLEGVLGMESAVVIIDDSVRVWPHNKLNLIVVERYIYFPCSRRQFGLPGPSLLEIDHDERPEDGTLASCLGVIQRIHQNFFAHRSIDEADVRNILATEQKKILAGCRIVFSRVFPVGEANPHLHPLWQTAEQFGAVCTSQIDDQVTHVVANSLGTDKVNWALSTGRFVVHPGWVEASALLYRRANEHDFAIKS

SEQ ID NO: 14 - protein, 1252 aa, Beta vulgaris
MKVSGGEDGEIPESDAIVEMKHQSKTTTTTTTTITSDSSNKKDNNSYWMRDLYKYSSYRGYGAASGLYNLAWAQAVQNKPLNEVLVELDDKKNNKNASTDDTSVNKEQGEVQQHCVESKEVFEVVDSEKEEGELEEGEIDFDSDDTGNNHNSNGNKVQDDFGGLEMDDGELENQVSSIRKVLHNVTVAEAHKSFDIVCARLRTSLETLRELVLHTWFPSKDALIQQAFAAIQCVYSVYSSMSPTLRDQNKDRMSRLLTFVMDLSSVLFTPEQRKEVEGMITSVNPPIVPVKPKSRDRQEELPVTEKAILTDSNTLTVNTGDNKSDLLKKVGPELSVYQSEKKNTDILSEAMRHFPSSLKVRSSFGPLLDLHKVHDEDSLPSPTSKTMPSLPFFETAPPRVVHGLQKSGVHPYETEAVKAVSSYQQRFGRSTFLATDMLPSPTPSEDGNEGGADDSNEEVSSSNAYTNVVSRTTNSSVVPQPVVSSAAYTSSSTMQGVISGTSAESSSVGSSPSLRASAKSRDPRLRHLNPNFGSLDLSFCPSPMVPSSASKLEPLGEIMKSKKTKALEGRLLDGPTAKRPRNGLETEDMSMNANQVKTLQGSTRMETSSSSILGPQSSSRGLLGPAIDPRKPGSGTVSSGITTNNPSMAVNKTAKPSMNVSGSPSLQSLLKDIAGNPGAWMNIIKEQNKSSEPLQSVSHSMNSNSILGAAPSAIAVPPISSGVGQTSAGLLQVPSPKVVTSSQDDSAKLRMKPRDPRRALHANMAQRTGSSVPEQPKVNGVHNTTTQGLQENINAQRYVNGTSPSAASSQTPILPDITKQFTKNLKNIADIISSPQTSSIQSPLAVSSLSAQANSDTTSISSGGQASCSSGGPVITGNQRTVSALRPEEVVSGRPQSQNNWGDVEHLFDGYDDQQKAAIQQERARRLDEQNKMFADRKLCLVLDLDHTLLNSAKFSEVDPVHDEILRKKEEQDREKPRRHLFRFPHMAMWTKLRPGIWNFLEKASKLFELHLYTMGNKLYATEMAKVLDPKGTLFAGRVISRGDDGDPIDGDERVPKSKDLEGVMGMESSVVIIDDSARVWPHNKLNLIVVERYTYFPCSRKQFGLPGPSLLEIDHDERPEEGTLASSLAVIEKIHQNFFSHKSLDDVDVRNILGAEQRKILAGCRILFSRVFPVGEANPHLHPLWQTAEQFGAVCTNQLDEQVTHVVANSLGTDKVNWALSTKRFVVHPSWVEASALLYRRVNEQDFAIKT

SEQ ID NO: 15 - protein, 1252 aa, Triticum aestivum
MRVTLTPKDEDWLVVLMTRERPRSAVVAPGGDVFTAGGGGETSDGDSSESLEEISAADFKESSSGTAAPSASSQRSRVWMGYTMSRSYAPAFHSFAWAQAVQNKPLVPRPAADEDEVEHLVDTSDEEKEEGEIEEGEAVQSTSPPIKQPETIDLDSDAQDKSESVDMEQTRLAVEAADELDFDQRVGSILEELERLSIEEAEKSFEASCARLRSCFESLKPLFPESGSPMPMLDALVQQAFVGIDTITTVANSYAMPKREQNKNMLLKLLFHIKNRYSDMLALNQRDELDSRVRQLVFVDGEDNAGSNCSTKTVNVVVPSGQVPSDRLPVESGAANPPRGSSFPSWEIPANNRIVSPLLDLHADYDENSLPSPTRVSAPPFPVPKPIGFGVFPMAPDRYFSAERIDPSKNFLYPCVNDALKDVSSYRQKYGPTSTFASDDLPSPTPSDDGDKSGDKEGDIFGEVSSFSASNKSAPPSGNLMPASRPSAVISSNDSFAGGPPGYAKQIEQSVSGPSHALKPSAKSRDPRLRFLNRDSGGTADANIHVNLAEPNASKDGTLGGVVSDNSRKHKATGQPLMDETVLKRARESTGSPRDILVPPGRDGSNISSYSGDRVQSNKHTGLETKTARNPSIRTSSQLISNVSSIPDSTGTLQASQPNSVPQTSAAPIVSLPAVLKDIAVNPTVLMHWIQMEHQKRSASEPQPASGIISSGMINNVTAGMVIPPGNALKTAEVAHIPSYRPQATSQTASVNSQNDPGVIRMKARDPRRVLHNNTSQKNDTLNSDQAKSNGIALPAFQDSKDNLINRQQLAEQLQTTVLPSQPVSLSSIARQSTMSASKVDPVSNSQLAASSLIAPQESLVSVNRADPRVAAGQNDSNNAAPATTLGTRPPANQWGDLDDLLNGYDDQQKALIQKERARRIMEQHTMFSSRKLCLVLDLDHTLLNSAKFIEVDPIHEEILRKKEEQDWERSERHLFRFHHMQMWTKLRPGIWNFLEKASKLYELHLYTMGNKLYATEMAKVLDPSGTLFAGRVISRGGDGISRGGDGDTFDSDDRVPKSKDLDGVLGMESAVVIIDDSVRVWPHNKNNMIVVERYTYFPCSRRQFGLPGPSLLEIDRDERPEDGTLASSLAVIGRIHQNFFSHPNLNDADVRSILSSEQRRILAGCRIVFSRIFPVGEANPHLHPLWQTAEQFGAVCTNQIDDRVTHVVANSLGTDKVNWALQTGRFVVHPGWVEASALLYRRANEHDFAVK

SEQ ID NO: 16 - protein, 1243 aa, Triticum aestivum
MRVTLTPKDEDWLVELMTRERPRSAVVAPGGDVFTASGGGETSDGDSSESLEEISAADFKESSSGTAAASASSQRSRVWMGYTMSRSYAPAFHSFAWAQAVQNKPLVPRPADEDEVEHLVDASEEEKEEGEIEEGEAVQSTSPPIKQPEAIDLDSDAQDKSESVAMEQTPLAVEAADELDFDQRVGSILEELERLSIEEAEKSFEGSCVRLRSCFESLKPLFPESGSPMPMLDALVQQAFVGIDTITTVANSYAMPKREQNKNMLLKLLFHIKNRYSDMLALNQRDELDSRVRQLVFVDGEDHAGSNCSSKTVNLVVPSGQVPSDRLPVESGAANPLGGSSFPSWEIPANNRMVSPLLDLHADYDENSLPSPTRDSAPPFPVPKPIGFGLFPMAPDRYFSAERVDPSKKVLYPCVNDALKDVSSYRQKYGQTSTFASDDLPSPTPSDDGDKSGDKEGDIFGEVSSFSASNLIPASRPSAVISSNDSFAGGPPGYAKQIEQSVSGPSHALKPSAKSRDPRLRFLNRDSGGTADANRHVNLAEPNASKDGTLGGVVSDNSRKHKATGQPLTDETVLKRARESTGNPRDMQVPPGRDGSNISSYSGNRVQSNQHKGLETKAAGNPSIRTSSQLISNVSSIPDSTGTLQASQPNSVPQTSAAPIVSLPAVLKDIAVNPTVLMHWIQMEHQKWSASEPQPASGIISSGMINNVTAGMVIPPGNAPKTAEVAHIPSYRPQATSQTASVNSQNDPGVIRMKARDPRRVLHNNTSQKNDTPNSDQAKSNGIALPAFQDSKDNLINREQLAEQLQTTVLPSQPVSLSSIARQSTMSASKVDPVSNSQLAASSLIAPQETLVSVNRADPRVAAGQNDSNDAAPATTLGTRPPANQWGDLDDLLNGYDDQQKALIQKERARRIMEQHTMFSSRKLCLVLDLDHTLLNSAKFIEVDPIHEEILRKKEEQDRERSERHLFRFHHMQMWTKLRPGIWNFLEKASKLYELHLYTMGNKLYATEMAKVLDPSGTLFAGRVISRGGDGISRGGDGDTFDSDDRVPKSKDLDGVLGMESAVVIIDDSVRVWPHNKNNMIVVERYTYFPCSRRQFGLPGPSLLEIDRDERPEDGTLASSLAVIGRIHQNFFSHPNLNDADVRSILSSEQRRILAGCRIVFSRIFPVGEANPHLHPLWQTAEQFGAVCTNQIDDRVTHVVANSLGTDKVNWALQTGRFVVHPGWVEASALLYRRANEHDFAVK

SEQ ID NO: 17 - protein, 1252 aa, Triticum aestivum
MRVTLTPKDEDWLVVLMTRERPRSAVVAPGGDVFTAGGGGETSDGDSSESLEEISAADFKESSSGTAAASASAQRSRVWMGYTMSRSYAPAFHSFAWAQAVQNKPLVPRPVADEDEVEHLVDASDEEKEEGEIEEGEAVQSTSPPIKQPETIDLDSDAQDKSESVAMEQTPLAFEAADELDFDQRVGSILEELERLSIEEAEKSFEGSCARLRSCFESLKPLFPESGSPMPMLDALVQQAFVGIDTITTVANSYAMPKREQNKNMLLKLLFHIKNRYSDMLALSQRDELDSRVRQLVFVDGEDNAGSNCSTKTVNVVVQSGQVPSDRLPVESGAANPLRGSSFPSWEIPANNRMVSPLLDLHADYDENSLPSPTRDSAPPFPVPKPIGFGVFPMAPDRYFLAERVDPSKKVLYTCVNDALKDVSSYRQKYGQTSTFASDDLPSPTPSDDGDKSGDKEGDIFGEVSSFSASNKSAPPSGNLIPASRPSAVISSNDSFAGGPPGYAKQIEQSVSGPSHALKPSAKSRDPRLRFLNRDSGGTADANRHVNFAEPNASKDGTLGGVVSDNSRKHKATGQPLTGETVLKRARESTGNPRDMQVPPSRDGSNISSYSGDRVQSNQHKGLETKAAGNPSIRTSSQLISNVSSIPDSTGTLQASQPNSVPQTSAAPIVSLPAVLKDIAVNPTVLMHWIQMEHQKRSASEPQPASGIISSGMINNVTAGMVIPPGNALKTAEVAHIPSYRPQATSQTASVNSQNDPGVIRMKARDPRRVLHNNTSQKNDTPNSDQAKSNGITLPAFQDSKDNLINREQLAEQLQTTVLPSQPVSLSSIAGQSTMSASKVDPVSNSQLAASSLIAPQETLVSVNRADPRVAAGQNDSNDAAPATTLGTRPPANQWGDLDDLLNGYDDQQKALIQKERARRIMEQHTMFSSRKLCLVLDLDHTLLNSAKFIEVDPIHEEILRKKEEQDRERSERHLFRFHHMQMWTKLRPGIWNFLEKASKLYELHLYTMGNKLYATEMAKVLDPSGTLFAGRVISRGGDGISRGGDGDTFDSDDRVPKSKDLDGVLGMESAVVIIDDSVRVWPHNKNNMIVVERYTYFPCSRRQFGLPGPSLLEIDRDERPEDGTLASSLAVIGRIHQNFFSHPNLNDADVRSILSSEQRRILAGCRIVFSRIFPVGEANPHLHPLWQTAEQFGAVCTNQIDDQVTHVVANSLGTDKVNWALQTGRFVVHPGWVEASALLYRRANEHDFAVK

SEQ ID NO: 18 - protein, 1197 aa, Sorghum bicolor
MARERPRSTVVAAGGDLVTAPGGGEGSDGDSAGSIEEISADDFRKDSSSALGGPAAAAAAGQRSRSWVGPPAVGYMARNFGHAFNSFAWSQAVRNKPLGLQPPPASDEDEVEHAVDASDGEKEEGEIEEGEAVEAEASPARAQPETIDLDADADALEKSESLAGAVPASAAEEEEVNLDQRVGSILEELEMVSIEEAEKSFEGACGRLHTCFENLKPLFQELENGSPMAILEPLMQQAFIGIDTLTTVAISYNLPRSEQNKTTLLKSLFHIKNRYSDMLTPEQRDELDSRVRKLVFGEKDNVSDPSTSSGTNAINVLAPSGQVSSSGGLPFESGAANPFSSLPRLEVPAKRISPLLDLHADYDENSLPSPTRDNAPPFPVPKPIGFGAFPMVPEKLSFPERVEPAKNSLYPSLNDPLKAVSSYQQKYGQKSVFPSDDLPSPTPSGDEGKSADKGGDIFSEVSSFPVPKSIALPSTSQMPASQPSTVSSSGISYASGPPGFAKQIEQPVAGPNHAIKAASKSRDPRLRFLNRDSAGATDVNRRANFSELKDGNLGGASVGNRKHKAIDDPQVDENVLKRFRGGTANPRDLQPTGNPNQLMNIRAPTNSSGINMKTLQPPQTTAPHVSAAPAVPVPSMLLKDIAVNPTLLMHLIQMEHQKKSASETQGGMSSGMSNNGIAGMVFTPGNAPKTTEAAQVPSVRPQVPAQTPSLNSQNDGGILRMKPRDPRRILHNNVAQKSDAMVLEQVKTNGITQPDSQGTKDQTSSMPSQPTLPSSVARPFTNTKHVDPVSNSQLAATAIMAPTQQALGSINKVDPRLAVEQNGQNADATTTDASATELEATQPVSPWGNLDHLLDGYDDKQKALIQKERARRITEQHKMFSARKLCLVLDLDHTLLNSAKFIEVEPIHEEMLRKKEEQDRTLPERHLYRFHHMNMWTKLRPGIWNFLEKASNLFELHLYTMGNKLYATEMAKVLDPTGTLFAGRVISRGDDGDPFDSDERVPKSKDLDGVLGMESAVVIIDDSVRVWPHNRHNLIVVERYTYFPCSRRQFGLPGPSLLEIDRDERPEDGTLASSLAVIERIHHNFFSHPNLNEADVRSILASEQRRILAGCRIVFSRVFPVGDASPHLHPLWQTAEQFGAVCTNLVDDRVTHVVANSPGTDKVNWALSKGKFVVHPGWVEASALLYRRANEHDFAVK

SEQ ID NO: 19 - protein, 1234 aa, Zea mays
MRVTVTPKDEERLVDLMAREQPRSAVVAAGGDLVTAAGGGGEGSDRDSSGSIEEITADDFKKDSSSALGGAAAAAGPRSRSWVAPPAVGYMARNFRYAFNSFAWSQAVRNKPLGLQPPAPDDDEVEHAVDVSDGEKEEGEIEEGEAVEALASPAPAQPETIDLDSDAPEKSESVAIDGSASVVPVPAAEEEEVNLDQRVGSILEELEMVSIEEAEKSFEGACARLHTCFENLKPLFQELENGSPMAILEPLMQQAFIGIDTLTTVANLYNLPRREQNKTTLLKLLFHIKNRYSDMLTPEQREEMDSRVRKLVFGEKDNVSDPSTSCGTSAINVSAPSGQVSNTGGLPFESGAANLFSSLPRLEVPAKRNSPLLNLHADYDENSLPSPTRDNAPPFPALKPIGFGAFPMVPEKLSFLDRVEPTKNSLYPPLNDPLKAVSSYQQKYGQKSVYPSDDLPSPTPSGDEGKPADKGGDIFSDVSSFPVPKSIVLPSTSQMPASQPSTVSSSSISYASSTSQMAASQPITVSSSGISYASGPPGFAKQIEQSTAGPNHAIKAASKSRDPRLRFLNRDSAGATDVNWRANFSELKDGNLGGVSVGNRKQKAVDDPQVDDNALKRFRGGIANQRDMQPTGNPNQLMNIRAPTHSSSINMKTLQPPQTTAPHVSAAPAVPLPPMLLKDIAVNPALLMHLIQMEHQKKSASESQGGMSSGMTNNGIAGMVFTPGNAPKITEAAQVPSVRPQVPVQTPPLNSQNDGGIVRMKPRDPRRILHNNIAQKSDAMSLEQVKNNGTTQPDSQGTKDQTTPVPSQPALPSSIARPFSSAKHVDPVSNSQLAATAIMAPTQALSSVNKVDPRLAVEQNGQNADATTNGASATTLEATQPVSPWGDVDHLLDGYDDQQKALIQKERARRITEQHKMFSARKLCLVLDLDHTLLNSAKFIEVEPIHEEMLRKKEEQDRTLPERHLYRFHHMNMWTKLRPGIWNFLQKASNLFELHLYTMGNKLYATEMAKVLDPTGTLFAGRVISRGDDGDPFDSDERVPKSKDLDGVLGMESAVVIIDDSVRVWPHNRHNLIVVERYTYFPCSRRQFGLPGPSLLEIDRDERPEDGTLASSLAVIERIHHNFFSHPNLNEADVRSILASEQRRILTGCRIVFSRVFPVGDASPHLHPLWQTAEQFGAVCTNLVDDRVTHIVANSPGTDKVNWALSKGKFVVHPGVEASALLYRRANEHDFAVK

SEQ ID NO: 20 - protein, 1241 aa, Arabidopsis thaliana
MLVARSGCSRTLIRMGNDENLMVMVDVEEGEIPDSVNTEIEVKHKSTTTTADVGGDVDVGVVAGGRGGGGGGSNGNSRVWTMEELISQYPAYRPYANSGLSNLAWARAVQNKPFNEGLVMDYEPRESDKIVIEDSDDEKEEGELEEGEIDLVDNASDDNLVEKDTESVVLISADKVEDDRILKERDLEKKVKLIRGVLESTSLVEAQTGFEGVCSRILGALESLRELVSDNDDFPKRDTLVQLSFASLQTINYVFCSMNNISKERNKETMSRLLTLVNDHFSQFLSFNQKNEIETMNQDLSRSAIAVFAGTSSEENVNQMTQPSNGDSFLAKKLTSESTHRGAAYLRSRLPMLPLLDLHKDHDADSLPSPTRETTPSLPVNGRHTMVRPGFPVGRESQTTEGAKVYSYESDARKAVSTYQQKFGLNSVFKTDDLPSPTPSGEPNDGNGDVGGEVSSSVVKSSNPGSHLIYGQDVPLPSNFNSRSMPVANSVSSTVPPHHLSIHAISAPTASDQTVKPSAKSRDPRLRLAKPDAANVTIYSYSSGDARNLSKVELSADLVNPRKQKAADEFLIDGPAWKRQKSDTDAPKAAGTGGWLEDTESSGLLKLESKPRLIENGVTSMTSSVMPTSAVSVSQKVRTASTDTASLQSLLKDIAVNPTMLLNLLKMGERQKVPEKAIQKPMDPRRAAQLPGSSVQPGVSTPLSIPASNALAANSLNSGVLQDSSQNAPAAESGSIRMKPRDPRRILHGSTLQRTDSSMEKQTKVNDPSTLGTLTMKGKAEDLETPPQLDPRQNISQNGTSKMKISGELLSGKTPDFSTQFTKNLKSIADMVVVSQQLGNPPASMHSVQLKTERDVKHNPSNPNAQDEDVSVSAASVTAAAGPTRSMNSWGDVEHLFEGYDDIQRVAIQRERVRRLEEQNKMFASQKLSLVLDIDHTLLNSAKFNEVESRHEEILRKKEEQDREKPYRHLFRFLHMGMWTKLRPGIWNFLEKASKLYELHLYTMGNKLYATEMAKLLDPKGVLFNGRVISKGDDGDPLDGDERVPKSKDLEGVMGMESSVVIIDDSVRVWPQHKMNLIAVERYLYFPCSRRQFGLLGPSLLELDRDEVPEEGTLASSLAVIEKIHQNFFSHTSLDEVDVRNILASEQRKILAGCRIVFSRIIPVGEAKPHLHPLWQTAEQFGAVCTTQVDEHVTHVVTNSLGTDKVNWALTRGRFVVHPGWVEASAFLYQRANENLYAINP

SEQ ID NO: 21 - DNA, 206 bp, synthetic construct
  misc_feature 1..206: note = silencing constructs TaCPL3_fragA
  source 1..206: mol_type = other DNA
gaaccgtgattctggtggtactgcagatgcaaatagacatgtaaatttggcagagccaaatgcttccaaagatgggaccttggggggtgttgtatcagataatagccggaagcacaaggcaactggccaacctctcacggatgaaaccgtgttaaaaagagctagggagagtactgggaatcccagagacatgcaggtaccaccta

SEQ ID NO: 22 - DNA, 201 bp, synthetic construct
  misc_feature 1..201: note = silencing constructs TaCPL3_fragB
  source 1..201: mol_type = other DNA
agcttctaatctgatacctgcctcccgacctagtgcagttatcagcagcaatgacagttttgcaggtggtcctccaggctatgctaaacaaattgaacagtctgtttcaggacccagccatgctcttaagccttcagctaaaagtagagatccaaggctcaggtttttgaaccgtgattctggtggtactgcagatgcaaa

SEQ ID NO: 23 - DNA, 201 bp, Zea mays
  source 1..201: mol_type = unassigned DNA
gaaggctgtctcttcctatcagcagaagtatgggcagaaatctgtatatccaagtgatgatctaccaagtccaactccatctggtgatgagggtaaacctgcagataaaggtggtgatatatttagtgatgtttccagcttccctgttccaaagagtattgtattaccaagtacaagtcagatgcctgcttctcaacctag

SEQ ID NO: 24 - DNA, 12867 bp, synthetic construct
  misc_feature 1..12867: note = p7U-70Subiintron-ZmCPL3_RNAi
  source 1..12867: mol_type = other DNA
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctgtttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtgcagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagtagtctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgagtggccaccatgggcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacgcgttctagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagctgatccactagaggccatggcggccgcgtcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagtcgacacgcgtaagcttgaattcctgcagcccgggggattggaaggctgtctcttcctatcagcagaagtatgggcagaaatctgtatatccaagtgatgatctaccaagtccaactccatctggtgatgagggtaaacctgcagataaaggtggtgatatatttagtgatgtttccagcttccctgttccaaagagtattgtattaccaagtacaagtcagatgcctgcttctcaacctagcgagaggtatggatagtgtatcttcatactgcatttgtttaatttgaaaatggttatctagttgcctaacaaaatatagctgggatatcatataacacatgtgcaggtgacatggaaaaaaatgcctatttttctatgcactaactattcatcatgtgacatacttccccaaaaaactaaataagccaaattttccagcttccgagtcctgaaaaagagtagtgtacctgatacaatttatagagtttttttttcgaaaagaagggatggccctcatagatagagtactaactaaaagtctacttttaccaatttcaggcctcgctaggttgagaagcaggcatctgacttgtacttggtaatacaatactctttggaacagggaagctggaaacatcactaaatatatcaccacctttatctgcaggtttaccctcatcaccagatggagttggacttggtagatcatcacttggatatacagatttctgcccatacttctgctgataggaagagacagccttccaatccccgggtacctctagacttgtacagctcgtccatgccgtacaggaacaggtggtggcggccctcggagcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacctgcatagtaaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccg

SEQ ID NO: 25 - DNA, 21 bp, synthetic construct
  misc_feature 1..21: note = M7GEP59
  source 1..21: mol_type = other DNA
ctcgtccttgggcgtgaccgt

SEQ ID NO: 26 - DNA, 21 bp, synthetic construct
  misc_feature 1..21: note = m7GEP60
  source 1..21: mol_type = other DNA
gtcactgctgccgggggcggg

SEQ ID NO: 27 - DNA, 21 bp, synthetic construct
  misc_feature 1..21: note = m7GEP61
  source 1..21: mol_type = other DNA
gctatgccttcaatagctttg

SEQ ID NO: 28 - DNA, 21 bp, synthetic construct
  misc_feature 1..21: note = m7GEP62
  source 1..21: mol_type = other DNA
cgtggtcgcaggccgtgcgga

SEQ ID NO: 29 - DNA, 21 bp, synthetic construct
  misc_feature 1..21: note = m7GEP63
  source 1..21: mol_type = other DNA
gactccgacgccccggagaag

SEQ ID NO: 30 - DNA, 21 bp, synthetic construct
  misc_feature 1..21: note = m7GEP64
  source 1..21: mol_type = other DNA
aggtgtctgagaaaaccagtt

SEQ ID NO: 31 - DNA, 21 bp, synthetic construct
  misc_feature 1..21: note = m7GEP65
  source 1..21: mol_type = other DNA
tcagacacctgaaacaaagcc

SEQ ID NO: 32 - DNA, 42 bp, Zea mays
  source 1..42: mol_type = unassigned DNA
tagccccagcggcttattccgcacggcctgcgaccacgcaaa

SEQ ID NO: 33 - DNA, 34 bp, synthetic construct
  misc_feature 1..34: note = Event GEZM054-T811
  source 1..34: mol_type = other DNA
tagccccagcggcttcggcctgcgaccacgcaaa

SEQ ID NO: 34 - DNA, 38 bp, synthetic construct
  misc_feature 1..38: note = Event GEZM054-T821
  source 1..38: mol_type = other DNA
tagccccagcggcttattccggcctgcgaccacgcaaa

SEQ ID NO: 35 - DNA, 26 bp, synthetic construct
  misc_feature 1..26: note = Event GEZM054-T868
  source 1..26: mol_type = other DNA
tagccccagcggtgcgaccacgcaaa

SEQ ID NO: 36 - DNA, 40 bp, synthetic construct
  misc_feature 1..40: note = Event GEZM054-T868
  source 1..40: mol_type = other DNA
tagccccagcggcttattccacggcctgcgaccacgcaaa

SEQ ID NO: 37 - DNA, 32 bp, synthetic construct
  misc_feature 1..32: note = Event GEZM054-T929
  source 1..32: mol_type = other DNA
tagccccagcggcttgcctgcgaccacgcaaa

SEQ ID NO: 38 - DNA, 34 bp, synthetic construct; identical to SEQ ID NO: 33
  misc_feature 1..34: note = Event GEZM054-T929
  source 1..34: mol_type = other DNA

SEQ ID NO: 39 - DNA, 28 bp, synthetic construct
  misc_feature 1..28: note = Event GEZM054-T941
  source 1..28: mol_type = other DNA
tagccccagcggcctgcgaccacgcaaa

SEQ ID NO: 40 - DNA, 31 bp, synthetic construct
  misc_feature 1..31: note = Event GEZM054-T1207
  source 1..31: mol_type = other DNA
tagccccagcggcggcctgcgaccacgcaaa

SEQ ID NO: 41 - DNA, 38 bp, synthetic construct; identical to SEQ ID NO: 34
  misc_feature 1..38: note = Event GEZM054-T1207
  source 1..38: mol_type = other DNA

SEQ ID NO: 42 - DNA, 25 bp, synthetic construct
  misc_feature 1..25: note = Event GEZM054-T1260
  source 1..25: mol_type = other DNA
tagccccggcctgcgaccacgcaaa

SEQ ID NO: 43 - DNA, 38 bp, synthetic construct; identical to SEQ ID NO: 34
  misc_feature 1..38: note = Event GEZM054-T1260
  source 1..38: mol_type = other DNA

SEQ ID NO: 44 - DNA, 37 bp, synthetic construct
  misc_feature 1..37: note = Event GEZM054-T1305
  source 1..37: mol_type = other DNA
tagccccagcggcttattccgcctgcgaccacgcaaa

SEQ ID NO: 45 - DNA, 31 bp, synthetic construct; identical to SEQ ID NO: 40
  misc_feature 1..31: note = Event GEZM054-T592
  source 1..31: mol_type = other DNA

SEQ ID NO: 46 - DNA, 32 bp, synthetic construct; identical to SEQ ID NO: 37
  misc_feature 1..32: note = Event GEZM054-T602
  source 1..32: mol_type = other DNA

SEQ ID NO: 47 - DNA, 25 bp, synthetic construct
  misc_feature 1..25: note = Event GEZM054-T626
  source 1..25: mol_type = other DNA
tagccccagcggcttaccacgcaaa

SEQ ID NO: 48 - DNA, 998 bp, Zea mays
  source 1..998: mol_type = unassigned DNA
atcctctcttttcttcgcccaaatctcgcgtgctgcctccgtcctgcccccagcgcgtgcgctccgatccgccgggtttcagcgctgcggctgtcgatttacgcgatttgtctggcagaatacagttcagagcctcgagcgctgcagatcgaggacctggtcgtcggattcgggcgccgcgggtgagacctaggcgccggagtttttgtgacggagttttggcgtggttggtggagggggggaggaaggagcggtctcatatgcgcgtgacggtcacgcccaaggacgaggaaaggctggttgacttgatggcgcgcgagcagccgcggtcggcggtggttgcggcgggcggagatttggtcactgctgccgggggcgggggcgaggggtcggatagggactcttcggggtccatagaggagattacggctgacgacttcaagaaggactcgtcgtcggcgttgggcggcgcggcggcggcggcggggccgagatctaggtcttgggtggccccgcccgccgtgggttacatggcccggaactttcgctatgccttcaatagctttgcgtggtcgcaggccgtgcggaataagccgctggggctacaaccgcctgccccagacgacgacgaggtggagcacgccgtggacgtctccgacggacagaaggaagagggcgagattgaggagggggaggctgtggaggccttcgcctcgcctgctcctgcgcagcctgagaccatcgatttggactccgacgccccggagaagtcagagtcggtggctatcgatggaagtgccagtgttgtgcctgtcccagctgctgaggaagaggaggtgaaccttgatcagcgtgtggggagtatactggaggagctcgagatggtctccattgaggaagctgagaagtatatgggcatatgcttcatgtttttccttgaacaaaggctttgtttcaggtgtctgagaaaaccagttgtaaccgctgccatgttgcag

SEQ ID NO: 49 - protein, 7 aa, synthetic construct
YSPTSPS

(19) United States
(12) Patent Application Publication
(10) Pub. No.: US 2025/0027104 A1
(43) Pub. Date: Jan. 23, 2025

(54) PATHOGEN RESISTANCE IN CROP PLANTS

(71) Applicant: KWS SAAT SE & Co. KGaA, Einbeck (DE)

(72) Inventors: Daniel Fabian STIRNWEIS, Gottingen (DE); Dietmar STAHL, Einbeck (DE); Urs Konrad FISCHER, Gottingen (DE); Christine KLAPPRODT, Osterode (DE)

(73) Assignee: KWS SAAT SE & Co. KGaA, Einbeck (DE)

(21) Appl. No.: 18/605,182

(22) Filed: Mar. 14, 2024

Related U.S. Application Data
(63) Continuation of application No. 17/433,316, filed on Aug. 24, 2021, now Pat. No. 11,932,866.

(30) Foreign Application Priority Data
Mar. 1, 2019 (EP) 19160408.1

(51) Int. Cl. C12N 15/82

(52) U.S. Cl. CPC C12N 15/8282

(57) ABSTRACT

The present invention relates to the field of plant biotechnology. Specifically, there are provided methods and nucleic acid sequences for obtaining pathogen resistance in plants and for generating resistant plants. In particular, the role of a central molecule in plant immunity is studied. Based on the mechanisms elucidated, the present invention provides strategies to specifically modulate said phosphatase-like protein family member molecule by different transient and/or stable techniques, alone or in combination, to achieve a robust increased of pathogen resistance in different target plants to obtain inherently pathogen resistant plants and plant materials to avoid severe harvest losses as caused by major plant pathogens by biological means instead of herbicide or pesticide treatment.

### CROSS-REFERENCE TO RELATED APPLICATIONS

This application is a continuation of U.S. application Ser. No. 17/433,316, filed Aug. 24, 2021, which is a U.S. National Phase of International Patent Application No. PCT/EP2020/055380, filed on Mar. 1, 2020, which claims priority to European Application No. 19160408.1, filed Mar. 1, 2019. The entire contents of these applications are incorporated herein by reference in their entirety.

### SEQUENCE LISTING

The instant application contains a Sequence Listing which has been submitted electronically in XML file format and is hereby incorporated by reference in its entirety. Said XML copy, created on Jan. 18, 2024, is named 245761_000231_SL.xml and is 121,174 bytes in size.

### Technical Field

The present invention relates to the field of plant biotechnology. Specifically, there are provided methods and nucleic acid sequences for obtaining pathogen resistance in plants and for generating resistant plants. In particular, the role of a central molecule in plant immunity is studied. Based on the mechanisms elucidated, the present invention provides strategies to specifically modulate a phosphatase-like protein family member molecule or the expression of said phosphatase-like protein family member molecule by different transient and/or stable techniques, alone or in combination, to achieve a robust increase of pathogen resistance in different target plants to obtain inherently pathogen resistant plants and plant materials to avoid severe harvest losses as caused by major plant pathogens by biological means instead of herbicide or pesticide treatment.

### Background of the Invention

Plant, unlike mammals, lack an immune system relying on mobile circulating defending cells and do not possess mechanisms like an adaptive immune system. Still, plants possess a highly efficient, two-layered innate immune system that allows them to respond to infections, e.g., by microbial pathogens (Jones and Dangl, (2006). The plant immune system. Nature, 444 (7117), 323). The first layer of defense relies on the recognition of evolutionary conserved pathogen- or microbial-associated molecular patterns (PAMPs or MAMPs) by the so-called pattern recognition receptors (PRRs). PAMPs or MAMPs are invariant structures broadly represented among microbial taxa and have essential roles in microbial physiology. Only an extremely selective group of molecules have been found to function as PAMPs (Gaudet and Gray-Owen, (2016). Heptose sounds the alarm: Innate sensing of a bacterial sugar stimulates immunity. PLoS pathogens, 12(9), e1005807). It was also found that conserved molecules from nematodes can elicit plant defenses and pathogen resistance (Manosalva et al., (2015). Conserved nematode signaling molecules elicit plant defenses and pathogen resistance. Nature communications, 6, 7795.). Accordingly, such molecules were defined as nematode-associated molecular patterns (NAMPs).

Besides molecular patterns originating from the pathogen, plants can also sense molecular patterns associated with cell wall destruction or cell damage, the so-called danger/damage-associated molecular patterns (DAMPs).

PRRs are generally plasma membrane receptors which are often coupled to intracellular kinase domains or require a co-receptor to provide signaling function. Depending on the presence of the signaling transduction domain the plant PRRs are classified either as receptor-like kinases (RLKs) or as receptor-like proteins (RLPs) as described by Macho and Zipfel ((2014). Plant PRRs and the activation of innate immune signaling. Molecular cell, 54(2), 263-272.). Recognition of PAMPs, MAMPs, NAMPs or DAMPs in the apoplast by pattern recognition receptors (PPRs) initiates a complex signaling cascades from the receptor in the plasma membrane to the nucleus leading to PRR-triggered immunity (PTI). As an evolutionary adaption, pathogens compete with the defense system and may be able to suppress the first defense layer through the secretion of effector proteins that interfere with the signaling (Jones and Dangl, 2006).

The second layer of plant defense, the effector triggered immunity (ETI), largely takes place inside a plant cell. ETI relies on the specific recognition of disease resistance effectors on a pathogen, wherein the disease resistance effector, as it is understood to date, usually are recognized by plant recognition proteins comprising polymorphic nucleotide binding (NB) and leucine rich repeat (LRR) domains. This recognition leads to a strong defense response which is often associated with a local programmed cell death, the hypersensitive reaction. Since pathogenic effectors are often species or isolate specific, this second layer of immunity is only efficient against isolates that carry the recognized effector, which is then called an avirulence gene.

Whether a potential pathogen is able to overcome the first layer of defense, the PTI, and to reproduce effectively, depends on its intrinsic ability to suppress PTI responses of the plant. But it also depends on the plants ability to efficiently and quickly induce and, if required, to maintain defense responses above a certain threshold for effective resistance (Jones and Dangl, 2006). PTI responses are generally conserved and include the activation of mitogen-activated protein kinases (MAPKs), the generation of reactive oxygen species, the activation of salicylic acid (SA)- and jasmonic acid (JA)-signaling pathways and the enhanced expression of plant defense genes, like pathogenesis-related genes.

Transcriptional activation as induced by a PTI or ETI mechanism can be measured very fast, typically within minutes or hours after infection and it is reduced after effective defense response. Transcription of protein-coding genes in eukaryotes is intricately orchestrated by RNA polymerase II (RNAPII), general transcription factors, mediators, and gene-specific transcription factors. The multi-subunit RNAPII complex is evolutionary conserved from yeast to human. Its largest subunit Rpb1 contains a carboxyl-terminal domain (CTD) consisting of conserved heptapeptide repeats with the consensus sequence Y₁S₂P₃T₄S₅P₆S₇ (SEQ ID NO: 49) (Buratowski, (2009). Progression through the RNA polymerase II CTD cycle. Molecular cell, 36(4), 541-546.). The number of repeats varies from 26 in yeast, 34 in Arabidopsis, and 52 in mammals (Hajheidari et al., (2013). Emerging roles for RNA polymerase II CTD in Arabidopsis. Trends in plant science, 18 (11), 633-643.). The combinatorial complexity of CTD posttranslational modifications constitutes the so-called “CTD-code” which is read by CTD-binding proteins to regulate the transcription cycle, modify chromatin structure, and modulate RNA capping, splicing and polyadenylation. In particular, the CTD undergoes waves of SER phosphorylation and dephosphorylation events regulated by various CTD kinases, often members of cyclin-dependent kinases (CDKs), and phosphatases during transcription initiation, elongation and termination. The interplay between different CTD kinases and phosphatases provides means for coupling and coordinating specific stages of transcription by recruiting other factors required for proper gene expression (Buratowski, 2009).

In Arabidospis, so far five members of the CTD phosphatase-like proteins family (CPL1-5) have been described (Koiwa et al. (2002). C-terminal domain phosphatase-like family members (AtCPLs) differentially regulate Arabidopsis thaliana abiotic stress signaling, growth, and development. Proceedings of the National Academy of Sciences, 99(16), 10893-10898.; and Fukudome et al. (2014). Arabidopsis CPL4 is an essential Ser2-specific CTD-phosphatase regulating general and xenobiotic responsive gene expression (617.2). The FASEB Journal, 28(1_supplement), 617-2.). They were shown to possess preferences for different phosphorylated serines in the heptapeptide repeats and to be involved in different biological processes. Arabidopsis AtCPL1 was shown to be a negative regulator of stress-responsive gene expression under various stress conditions (cold, abscisic acid (BA), salt treatment and iron deficiency) (Koiwa et al., 2002) and negatively regulates wound-induced JA-biosynthesis genes. Arabidopsis AtCPL2 was described to be involved in the regulation of osmotic stress and auxin responses and influences plant development (Ueda et al., (2008). The Arabidopsis thaliana carboxyl-terminal domain phosphatase-like 2 regulates plant growth, stress and auxin responses. Plant molecular biology, 67(6), 683.). Arabidopsis AtCPL3 was shown to be a negative regulator of BABA-induced gene expression (Koiwa et al., 2002). A complete knockout of AtCPL3 by T-DNA insertion or early-stop-codon mutations resulted in reduced growth and early flowering (Koiwa et al., 2002). Arabidopsis AtCPL4 was described as an essential gene involved in the regulation of xenobiotic stress responses (Fukudome et al., 2014), lateral root development and its silencing induces cytokinin-oversensitive de novo shoot organogenesis (Fukudome et al., (2018). Silencing Arabidopsis CARBOXYL-TERMINAL DOMAIN PHOSPHATASE-LIKE 4 induces cytokinin-oversensitive de novo shoot organogenesis. The Plant Journal, 94(5), 799-812.). Arabidopsis AtCPL5 was reported to encode a unique CPL family protein that positively regulates ABA-mediated development and drought responses in Arabidopsis (Jin et al., (2011). AtCPL5, a novel Ser-2-specific RNA polymerase II C-terminal domain phosphatase, positively regulates ABA and drought responses in Arabidopsis. New Phytologist, 190 (1), 57-74.). At date, certain CPL family members of genes/proteins has been identified in Arabidopsis, but further work on the specific function of said proteins, in particular in the context of the complex network of plant immunity, has to be accomplished.

Presently, there is thus an increasing understanding of plant immunity. In particular, certain aspects of PRR signaling and ETI responses in plants have been elucidated, yet only very few is known about the complex interplay between plants and pathogens seeking to subvert the plant immune system in a co-evolutionary manner. Notably, depending on its life cycle and mode of infection, each pathogen will have an individual defense strategy so that individual defense mechanisms are needed for sessile plants to combat infection.

Infections and infestations of crop plants by pathogens encompassing viruses, bacteria, fungi, nematodes and insects and the resulting damages cause significant yield losses of cultivated plants. In maize or corn (Zea mays), said terms being used interchangeably herein, as one of the major crop plants worldwide there are a large number of fungal pathogens which cause leaf diseases. The fungus which can cause by far the most damage under tropical and also under temperate climatic conditions, such as those in large parts of Europe and North America as well as in Africa and India, is known as Helminthosporium turcicum or synonymously as Exserohilum turcicum (teleomorph: Setosphaeria turcica). H. turcicum/E. turcicum is the cause of the leaf spot disease known as “Northern Corn Leaf Blight” (NCLB), which can occur in epidemic proportions during wet years, attacking vulnerable maize varieties and causing a great deal of damage and considerable losses of yield of 30% and more over wide areas (Perkins & Pedersen, 1987. Disease development and yield losses associated with northern leaf blight on corn. Plant Disease, 71(10), 940-943.; Raymundo & Hooker, 1981. Effect of gene HtN on the development of northern corn leaf blight epidemics. Plant disease.). Since the 1970s, natural resistance in genetic material has been sought. Of course, it is not only fungi that cause plant diseases. There are also bacteria, viruses, nematode worms (e.g., eel worms), aphids and insects to name relevant further plant pathogens. Serious plant diseases are caused by all these other pests, but fungi probably cause the most severe losses for major crop plants worldwide. Crop protection measures include weed control, which can be managed mechanically or chemically, and the control of animal pests or diseases, which relies heavily on synthetic chemicals. Herbicide use has enabled farmers to modify production systems to increase crop productivity while still maintaining some measure of control over the damaging effect of pests. Unfortunately, despite large increases in pesticide use, crop losses have not significantly decreased during the last 40 years.

The race for defining and establishing new resistance strategies against pathogens for major crop plants is more and more accelerated due to the increasing resistance breaking characteristics of pathogens, i.e., the evolutionary strategy of pathogens to adapt to and survive pressure of plant protective agents and/or to subvert the endogenous plant immunity defence mechanisms introduced above.

So far, there is thus a great need to transfer the basic knowledge about plant defense mechanisms and plant immunity elucidated in model plants like Arabidopsis thaliana to major crop plants. It was therefore an intention of the present invention to identify new genes specifically present in crop plants that could be associated with pathogen resistance. It was another object of the present invention to transfer the knowledge about conserved signaling mechanisms in plant immunity to a more specific level to identify specific interactions and to present new strategies to establish broad pathogen resistance in a variety of crop plants based on biological means, i.e., disease control strategies relying on establishing and growing resistant cultivars for providing more effective and environmentally sound disease control, wherein obtaining plant varieties with greater pathogen resistance is central to this.

It was thus a further object of the present invention to investigate the functional basis of resistance to pathogens, including fungal pathogens as well as further kinds of plant pathogens, to provide new strategies to combat pathogen infection or infestation based on exploiting plant-endogenous defense mechanisms as source of resistance or tolerance against pathogens. Instead of relying on anti-fungal chemicals, it was an aim to establish new urgently needed resistance strategies for a variety of important crop plants by characterizing the molecular players involved in disease resistance in a plant and in turn to modulate said plant-endogenous resistance pathways in a targeted manner, in particular to obtain increased pathogen resistance whilst maintaining normal growth in a plant in view of the fact that modulating central effectors of the plant immune system without a proper understanding of the regulatory mechanisms is usually accomplished with severe side effects, e.g., reduced plant development or growth which should be avoided according to the present invention.

### SUMMARY OF THE INVENTION

The above object was achieved by identifying the molecular role of CPL genes, in particular CPL3, in pathogen resistance and further by studying the effect of targeted mutations or modulations of said genes or gene products or their expression or translation, which has advantageous effects in comparison to the generation of full knock-out lines.

In a first aspect, there is provided a plant having pathogen resistance, wherein pathogen resistance is conferred or increased by modulation of a nucleotide sequence encoding an endogenous C-terminal domain phosphatase-like 3 (CPL3) protein or a regulatory sequence thereof, or by modulation of the transcription of an endogenous CPL3 protein, wherein modulation is achieved by (i) one or more mutation(s) of the nucleotide sequence encoding a CPL3 protein, preferably wherein the one or more mutation(s) has/have a dominant negative effect, preferably wherein the one or more mutation(s) cause(s) an alteration of the amino acid sequence of the conserved catalytic domain of the CPL3 protein comprising the DXDXT/V motif; and/or (ii) one or more silencing construct(s) directed to one or more endogenous nucleotide sequence(s) encoding a CPL3 protein, preferably directed to all endogenous nucleotide sequences encoding a CPL3 protein; and/or (iii) a modification of the native regulatory sequence(s) of one or more nucleotide sequence(s) encoding an endogenous CPL3 protein, preferably of all native regulatory sequence(s) of the nucleotide sequences encoding an endogenous CPL3 protein, wherein the modification causes a reduced expression rate of the one or more nucleotide sequence(s) encoding an endogenous CPL3 protein.

In a further aspect, there is provided a cell, tissue, organ, seed or material of the plant having pathogen resistance, wherein pathogen resistance is conferred or increased by modulation of a nucleotide sequence encoding an endogenous C-terminal domain phosphatase-like 3 (CPL3) protein or a regulatory sequence thereof, or by modulation of the transcription of an endogenous CPL3 protein as detailed in the above first aspect.

In a second aspect, there is provided a nucleic acid molecule comprising a nucleotide sequence encoding for a C-terminal domain phosphatase-like 3 (CPL3) protein, wherein the nucleotide sequence is selected from the group consisting of: (a) a nucleotide sequence set forth in SEQ ID NOs: 2-10 or a homologous, orthologous or paralogous sequence thereof; (b) a nucleotide sequence having at least 80%, at least 85%, at least 90%, at least 92%, at least 94%, at least 95%, at least 96%, at least 97%, at least 98% or at least 99% identity to one of the nucleotide sequences set forth in SEQ ID NOs: 2-10, (c) a nucleotide sequence encoding for the amino acid sequence set forth in SEQ ID NOs: 11-19; (d) a nucleotide sequence encoding for an amino acid sequence having at least 80%, at least 85%, at least 90%, at least 92%, at least 94%, at least 95%, at least 96%, at least 97%, at least 98% or at least 99% identity to one of the sequences set forth in SEQ ID NOs: 11-19, or (e) a nucleotide sequence hybridizing with a nucleotide sequence complementary to the nucleotide sequence as defined in (a)-(d) under stringent conditions, wherein the nucleotide sequence comprises at least one mutation capable of conferring or increasing resistance to a pathogen in plant in which the nucleic acid molecule is expressed, wherein the pathogen is at least one of a fungal pathogen, an oomycete pathogen, a bacterial pathogen, a virus, a nematode pathogen, or an insect, preferably wherein the pathogen is a hemibiotrophic fungus, more preferably the pathogen is a hemibiotrophic fungus selected from the group consisting of: Zymoseptoria tritici, Setosphaeria turcica, Fusarium spp. Fusarium graminearum, Colletotrichum spp. such as Colletotrichum graminicola, Magnaporthe grisea, Magnaporthe oryzae, Phytophthora infestans, or preferably wherein the pathogen is a fungus selected from Cercospora spp., preferably Cercospora beticola or Cercospora zeae-mayidis.

In yet another aspect, there is provided a method of conferring or enhancing the pathogen resistance in a plant or of generating a plant having pathogen resistance or a plant cell, tissue, organ, seed, or plant material thereof, comprising the steps of: (i) providing one or more silencing construct(s), or one or more sequences encoding the same; (ii) modifying a plant cell, tissue, organ, plant, seed, or plant material by introducing the one or more silencing construct(s) or the sequence encoding the same of (i), into the genome of said plant cell, tissue, organ, plant, seed, or plant material; and (iii) obtaining the modified plant cell, tissue, organ, plant, seed or plant material, (iv) optionally, regenerating a plant from the plant cell, tissue, organ or plant material or growing a seed on a plant obtained in (iii), wherein the plant cell, tissue, organ, plant, seed or plant material obtained in (iii), the plant regenerated in (iv) or the seed grown in (iv) comprise the introduced one or more silencing construct(s) or the sequence encoding the same and thereby has pathogen resistance.

In another aspect, there is provided a method of conferring or enhancing the pathogen resistance in a plant or of generating a plant having pathogen resistance or a plant cell, tissue, organ, seed, or plant material thereof, comprising the steps of: (i) providing at least one site-directed DNA modifying enzyme, or a sequence encoding the same, and optionally at least one DNA repair template, wherein the at least one site-directed DNA modifying enzyme and optionally the at least one DNA repair template: (a) is/are directed or targeted to the nucleotide sequence encoding the CPL3 protein; or (b) is/are directed or targeted to regulatory sequence of at least one CPL3 protein encoding nucleotide sequence; (ii) introducing the at least one site-directed DNA modifying enzyme or a sequence encoding the same, and optionally the at least one DNA repair template into the plant cell, tissue, organ, plant, or plant material; (iii) mutating or modifying the nucleotide sequence encoding the CPL3 protein or the regulatory sequence thereof in the genome of the plant cell, tissue, organ, plant, or plant material and obtaining a mutant or modified population of plant cells, tissues, organs, plants, or plant materials; (iv) optionally: screening the population for a dominant negative mutation, thereby conferring or increasing pathogen resistance, or screening the population for a mutation or modification in the nucleotide sequence encoding the CPL3 protein or the regulatory sequence thereof; (v) identifying and thereby obtaining a plant cell, tissue, organ, plant, or plant material having pathogen resistance.

In another aspect, there is provided a method of conferring or enhancing the pathogen resistance in a plant or of generating a plant having pathogen resistance or a plant cell, tissue, organ, seed, or plant material thereof, comprising the steps of: (i) subjecting the plant cell, tissue, organ, plant, or plant material, preferably seeds of a plant, to an efficient amount of a mutagenic agent, preferably ethylmethane sulfonate, N-ethyl-N-nitrosourea, or radiation, (ii) obtaining a mutagenized population of plant cells, tissues, organs, plants, or plant materials, optionally by growing plants from the mutagenized population; (iii) screening the mutagenized population for pathogen resistance, optionally by isolating and analyzing genomic DNA from the plants having pathogen resistance; (iv) identifying and obtaining a modified plant cell, tissue, organ, plant, or plant material having pathogen resistance.

In yet another aspect, there is provided a method of conferring or enhancing the pathogen resistance in a plant or of generating a plant having pathogen resistance or a plant cell, tissue, organ, seed, or plant material thereof, comprising the steps of: (i) transforming at least one plant cell with at least one nucleic acid molecule as defined in the second aspect above; and (ii) regenerating and thus obtaining a plant cell, tissue, organ, plant, or plant material having pathogen resistance.

In a further aspect there is provided a method for identifying a plant having pathogen resistance or a plant cell, tissue, organ, seed, or plant material thereof, comprising the steps of: (i) isolating DNA from at least one cell of the plant or of tissue, organ, seed, or plant material thereof, and (ii) detecting at least one nucleic acid molecule as defined in the second aspect above, and optionally (iii) selecting a plant comprising at least one nucleic acid molecule as defined in the second aspect above based on the detection in step (ii), and optionally (iv) breeding progeny having pathogen resistance through crossing of the plant selected in step (iii) with another plant, preferably of the same species, and thereby introducing the at least one nucleic acid molecule detecting in step (ii) in to the genome of the progeny.

Another aspects provides the use of the nucleic acid molecule as defined in the second aspect above, or the use of a silencing construct as defined in the first aspect above, for the generation of a plant cell, tissue, organ, whole plant, or plant material having pathogen resistance, or for conferring or increasing pathogen resistance of in a plant, plant cell, tissue, organ, whole plant, or plant material.

### Definitions

An “allele” or “allelic variant” as used herein refers to a variant form of a given gene. As most multicellular organisms have two sets of chromosomes; that is, they are diploid (or, if more chromosome sets are present, they are polyploidy), these chromosomes are referred to as homologous chromosomes. If both alleles at a gene (or locus) on the homologous chromosomes are the same, they and the organism are homozygous with respect to that gene (or locus). If the alleles are different, they and the organism are heterozygous with respect to that gene. Alleles can result in the same, or a different observable phenotype. The term “allele” thus refers to one or two or more nucleotide sequences at a specific locus in the genome. A first allele is on a chromosome, a second on a second chromosome at the same position. If the two alleles are different, they are heterozygous, and if they are the same, they are homozygous. Various alleles of a gene (gene alleles) differ in at least one SNP (single nucleotide polymorphism). Additionally, ploidy gives the number of complete sets of chromosomes in a cell, and hence the number of possible alleles. The generic term polyploid is used to describe cells with three or more chromosome sets. For example, about half of all known plant genera contain polyploid species, and about two third of all grasses are polyploid.

The term “anamorph” or “anamorphs” as used herein in the context of mycology refers to an asexual reproductive stage (morph), often mold-like of a fungus. When a single fungus produces multiple morphologically distinct anamorphs, these are called synanamorphs. The “teleomorph” form of the fungus represents the sexual reproductive stage (morph), typically a fruiting body. A “holomorph” means the whole fungus, i.e., including anamorphs and teleomorph.

The term “catalytically active fragment” or “functional fragment” as used herein referring to amino acid sequences denotes the core sequence derived from a given template amino acid sequence, or a nucleic acid sequence encoding the same, comprising all or part of the active site of the template sequence with the proviso that the resulting catalytically active fragment still possesses the activity characterizing the template sequence, for which the active site of the native enzyme or a variant thereof is responsible. Said modifications are suitable to generate less bulky amino acid sequences still having the same activity as a template sequence making the catalytically active fragment a more versatile or more stable tool being sterically less demanding. For amino acid sequences not representing enzymes, the term “functional fragment” can also imply that part or domain of the amino acid sequence involved in interaction with another molecule, and/or involved in any structural function within the cell.

“Complementary” or “complementarity” as used herein describes the relationship between two DNA, two RNA, or, regarding hybrid sequences according to the present invention, between an RNA and a DNA nucleic acid region. Defined by the nucleobases of the DNA or RNA, two nucleic acid regions can hybridize to each other in accordance with the lock-and-key model. To this end the principles of Watson-Crick base pairing have the basis adenine and thymine/uracil as well as guanine and cytosine, respectively, as complementary bases apply. Furthermore, also non-Watson-Crick pairing, like reverse-Watson-Crick, Hoogsteen, reverse-Hoogsteen and Wobble pairing are comprised by the term “complementary” as used herein as long as the respective base pairs can build hydrogen bonding to each other, i.e., two different nucleic acid strands can hybridize to each other based on said complementarity.

The term “construct”, “recombinant construct” or expression construct, especially also “silencing construct”, refers to a recombinant construct or expression construct and, as used herein, refers to a construct comprising, inter alia, plasmids or plasmid vectors, and may comprise an expression cassette, isolated single-stranded or double-stranded nucleic acid sequences, comprising DNA and/or RNA sequences, or amino acid sequences, viral vectors, including modified viruses, and a combination or a mixture thereof, for introduction or transformation, transfection or transduction into a target cell or plant, plant cell, tissue, organ or material according to the present disclosure.

The term “delivery construct” or “delivery vector” as used herein refers to any biological or chemical means used as a cargo for transporting a nucleic acid, comprising RNA and/or DNA, and/or an amino acid sequence of interest into a target cell, preferably a eukaryotic cell. The term delivery construct or vector as used herein thus refers to a means of transport to deliver a genetic or a recombinant construct according to the present disclosure into a target cell, tissue, organ or an organism. A vector can thus comprise nucleic acid sequences, optionally comprising sequences like regulatory sequences or localization sequences for delivery, either directly or indirectly, into a target cell of interest or into a plant target structure in the desired cellular compartment of a plant. A vector can also be used to introduce an amino acid sequence or a ribonucleo-molecular complex into a target cell or target structure. Usually, a vector as used herein can be a plasmid vector. Furthermore, a direct introduction of a construct or sequence or complex of interest can be conducted, e.g., by chemical means of transfection. The term “introduction” in this context shall imply both a direct and an indirect introduction. Direct introduction implies that the desired target cell or target structure containing a DNA target sequence to be modified according to the present disclosure is directly transformed or transduced or transfected into the specific target cell of interest, where the material delivered with the delivery vector will exert its effect. The term indirect introduction implies that the introduction is achieved into a structure, for example, cells of leaves or cells of organs or tissues, which do not themselves represent the actual target cell or structure of interest to be transformed, but those structures serve as basis for the systemic spread and transfer of the vector or construct to the actual target structure. In case the term vector is used in the context of transfecting amino acid sequences and/or nucleic sequences into a target cell the term vector implies suitable agents for peptide or protein transfection, like for example ionic lipid mixtures, cell penetrating peptides (CPPs), or particle bombardment. In the context of the introduction of nucleic acid material, the term vector cannot only imply plasmid vectors but also suitable carrier materials which can serve as basis for the introduction of nucleic acid and/or amino acid sequence delivery into a target cell of interest, for example by means of particle bombardment. Said carrier material comprises, inter alia, gold or tungsten particles. Viral vectors, as further detailed below, and bacterial vectors, like for example Agrobacterium spp., like for example Agrobacterium tumefaciens vectors can be used, e.g., binary and superbinary vectors. Finally, the term vector also implies suitable chemical transport agents for introducing linear nucleic acid sequences (single-or double-stranded), or amino sequences, or a combination thereof into a target cell combined with a physical introduction method, including polymeric or lipid-based delivery constructs. Suitable “delivery constructs” or “vectors” thus comprise biological means for delivering nucleotide and/or amino acid sequences into a target cell, including viral vectors, Agrobacterium spp., or chemical delivery constructs, including nanoparticles, e.g., mesoporous silica nanoparticles (MSNPs), cationic polymers, including PEI (polyethylenimine) polymer based approaches or polymers like DEAE-dextran, or non-covalent surface attachment of PEI to generate cationic surfaces, lipid or polymeric vesicles, or combinations thereof. Lipid or polymeric vesicles may be selected, for example, from lipids, liposomes, lipid encapsulation systems, nanoparticles, small nucleic acid-lipid particle formulations, polymers, and polymersomes.

The term “derivative” or “descendant” or “progeny” as used herein according to the present disclosure relates to the descendants of such a cell or material which result from natural reproductive propagation including sexual and asexual propagation. It is well known to the person having skill in the art that said propagation can lead to the introduction of mutations into the genome of an organism resulting from natural phenomena which results in a descendant or progeny, which is genomically different to the parental organism or cell, however, still belongs to the same genus/species and possesses mostly the same characteristics as the parental recombinant host cell. Such derivatives or descendants or progeny resulting from natural phenomena during reproduction or regeneration are thus comprised by the term of the present disclosure.

Furthermore, the terms “derived”, “derived from”, or “derivative” as used herein in the context of an isolated biological sequence (nucleic acid or amino acid) or a molecule or a complex—rather than referring to a whole cell or organism—may imply that the respective sequence is based on a reference sequence, for example from the sequence listing, or a database accession number, or the respective scaffold structure, i.e., originating from said sequence, whereas the reference sequence can comprise more sequences, e.g., the whole genome or a full polyprotein encoding sequence, of a virus, whereas the sequence “derived from” the native sequence, i.e. a sequence as naturally occurring in a cell or organism, may only comprise one isolated fragment thereof, or a coherent fragment thereof. In this context, a cDNA molecule or a RNA can be said to be “derived from” a DNA sequence serving as molecular template. The skilled person can thus easily define a sequence “derived from” a reference sequence, which will, by sequence alignment on DNA or amino acid level, have a high identity to the respective reference sequence and which will have coherent stretches of DNA/amino acids in common with the respective reference sequence (>75% query identity for a given length of the molecule aligned provided that the derived sequence is the query and the reference sequence represents the subject during a sequence alignment). The skilled person can thus clone the respective sequences based on the disclosure provided herein by means of polymerase chain reactions and the like into a suitable vector system of interest, or use a sequence as vector scaffold. The term “derived from” is thus no arbitrary sequence, but a sequence corresponding to a reference sequence it is derived from, whereas certain differences, e.g., certain mutations naturally occurring during replication of a recombinant construct within a host cell, cannot be excluded and are thus comprised by the term “derived from”. Furthermore, several sequence stretches from a parent sequence can be concatenated in a sequence derived from the parent. The different stretches will have high or even 100% identity to the parent sequence.

The term an “endogenous” in the context of nucleic acid and/or amino acid sequences refers to the nucleic acid and/or amino acid as found and/or expressed in a plant genome in its natural form as DNA/RNA or protein. As it is known to the skilled person, several variants, e.g., allelic variants, of a gene nucleic acid sequence may exist in a given population of plants.

A “fungus” or “fungal pathogen” as used herein means any plant pathogenic fungus in any developmental stage, including spores, or any part of such a fungus, which can interact with a plant or plant part or cell to induce a response in said plant or plant part or cell.

As used herein, “fusion”, e.g., in the context of a base editor or a CRISPR/Cas system, can refer to a protein and/or nucleic acid comprising one or more non-native sequences (e.g., moieties). A fusion can be at the N-terminal or C-terminal end of the modified protein, or both, or within the molecule as separate domain. For nucleic acid molecules, the fusion molecule can be attached at the 5′ or 3′ end, or at any suitable position in between. A fusion can be a transcriptional and/or translational fusion. A fusion can comprise one or more of the same non-native sequences. A fusion can comprise one 10 or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-specific effector or base editor (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as alexa fluor dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-galactosidase, and the like. A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles, or nanodiamonds, optionally linked to a nanoparticle. Any optical or magnetic property or characteristic of the nanoparticle(s) can be detected. A fusion can comprise a helicase, a nuclease (e.g., Fokl), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long non-coding RNA, a DNA repair protein (e.g., a protein involved in repair of either single-and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

The term “modification” or “genetic modification” is used in a broad sense herein and means any modification of a nucleic acid sequence or an amino acid sequence, a target cell, tissue, organ or organism, which is accomplished by human intervention, either directly or indirectly, to influence the endogenous genetic material or the transcriptome or the proteome of a target cell, tissue, organ or organism to modify it in a purposive way so that it differs from its state as found without human intervention. The human intervention can either take place in vitro or in vivo/in planta, or also both. Further modifications can be included, for example, one or more point mutation(s), e.g., for targeted protein engineering or for codon optimization, deletion(s), and one or more insertion(s) or deletion(s) of at least one nucleic acid or amino acid molecule (including also homologous recombination), modification of a nucleic acid or an amino acid sequence, or a combination thereof. The terms shall also comprise a nucleic acid molecule or an amino acid molecule or a host cell or an organism, including a plant or a plant material thereof which is/are similar to a comparable sequence, organism or material as occurring in nature, but which have been constructed by at least one step of purposive manipulation. The modification can be effected in a transient way, or in a stable, inheritable manner.

The term “genome” refers to the entire complement of genetic material, including genes and non-coding sequences, the nuclear and optionally present further genomes, e.g., the genome of organelles, that is present in each cell of an organism, or organelle, and/or a complete set of chromosomes inherited as a (haploid) unit from one parent, or that encodes a virus. The genome thus also defines the “genotype” being the part of the genetic makeup of a given cell, and therefore of an organism or individual, which determines a specific characteristic (phenotype) of that cell/organism/individual.

The terms “genome editing”, “genome engineering”, or “gene editing/engineering” are used interchangeably herein and refer to strategies and techniques for the targeted, specific modification of any genetic information or genome of a living organism. As such, the terms comprise gene editing, but also the editing of regions other than gene encoding regions of a genome. It further comprises the editing or engineering of the nuclear (if present) as well as other genetic information (e.g., the RNA transcriptome) of a cell. Furthermore, the terms “genome editing” and “genome engineering” also comprise an epigenetic editing or engineering, i.e., the targeted modification of, e.g., methylation, histone modification or of non-coding RNAs possibly causing heritable changes in gene expression.

“Germplasm”, as used herein, is a term used to describe the genetic resources, or more precisely the DNA of an organism and collections of that material. In breeding technology, the term germplasm is used to indicate the collection of genetic material from which a new plant or plant variety can be created.

The terms “guide RNA”, “gRNA” or “single guide RNA” or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. The tracr and the crRNA moiety thus do not necessarily have to be present on one covalently attached RNA molecule, yet they can also be comprised by two individual RNA molecules, which can associate or can be associated by non-covalent or covalent interaction to provide a gRNA according to the present disclosure. The terms “gDNA” or “sgDNA” or “guide DNA” are used interchangeably herein and either refer to a nucleic acid molecule interacting with an Argonaute nuclease. Both, the gRNAs and gDNAs as disclosed herein are termed “guiding nucleic acids” or “guide nucleic acids” due to their capacity to interacting with a site-specific nuclease and to assist in targeting said site-specific nuclease to a genomic target site.

The term “hemibiotroph” or “hembibiotrophic” refers to an organism that is in part (hemi) parasitic in living tissue for some time and thus relies on an initial biotrophic phase. This phase is followed by a necrotrophic phase, i.e., the organism can induce host cell death and/or can persist in dead tissue without the need for a living host or host cell.

The term “hybridization” as used herein refers to the pairing of complementary nucleic acids, i.e., DNA and/or RNA, using any process by which a strand of nucleic acid joins with a complementary strand through base pairing to form a hybridized complex. Hybridization and the strength of hybridization (i.e., the strength of the association between the nucleic acids) is impacted by such factors as the degree and length of complementarity between the nucleic acids, stringency of the conditions involved, the Tm of the formed hybrid, and the G:C ratio within the nucleic acids. The term hybridized complex refers to a complex formed between two nucleic acid sequences by virtue of the formation of hydrogen bounds between complementary G and C bases and between complementary A and T/U bases. A hybridized complex or a corresponding hybrid construct can be formed between two DNA nucleic acid molecules, between two RNA nucleic acid molecules or between a DNA and an RNA nucleic acid molecule. For all constellations, the nucleic acid molecules can be naturally occurring nucleic acid molecules generated in vitro or in vivo and/or artificial or synthetic nucleic acid molecules. Hybridization as detailed above, e.g., Watson-Crick base pairs, which can form between DNA, RNA and DNA/RNA sequences, are dictated by a specific hydrogen bonding pattern, which thus represents a non-covalent attachment form according to the present invention. In the context of hybridization, the term “stringent (hybridization) conditions” should be understood to mean those conditions under which a hybridization takes place primarily only between homologous nucleic acid molecules. The term “hybridization conditions” in this respect refers not only to the actual conditions prevailing during actual agglomeration of the nucleic acids, but also to the conditions prevailing during the subsequent washing steps. Examples of stringent hybridization conditions are conditions under which primarily only those nucleic acid molecules that have at least 70%, preferably at least 75%, at least 80%, at least 85%, at least 90% or at least 95% sequence identity undergo hybridization. Stringent hybridization conditions are, for example: 4×SSC at 65° C. and subsequent multiple washes in 0.1×SSC at 65° C. for approximately 1 hour. The term “stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2xSSC and 0.1% SDS at 68° C. Preferably, hybridization takes place under stringent conditions.

As used herein, the term “mutation” is used to refer to a deletion, insertion, addition, substitution, edit, strand break, and/or introduction of an adduct in the context of nucleic acid manipulation in vivo or in vitro. A deletion is defined as a change in a nucleic acid sequence in which one or more nucleotides is absent. An insertion or addition is that change in a nucleic acid sequence which has resulted in the addition of one or more nucleotides. A “substitution” or “edit” results from the replacement of one or more nucleotides by a molecule which is a different molecule from the replaced one or more nucleotides. For example, a nucleic acid may be replaced by a different nucleic acid as exemplified by replacement of a thymine by a cytosine, adenine, guanine, or uridine. Pyrimidine to pyrimidine (e.g., C to Tor T to C nucleotide substitutions) or purine to purine (e.g., G to A or A to G nucleotide substitutions) are termed transitions, whereas pyrimidine to purine or purine to pyrimidine (e.g., G to T or G to C or A to T or A to C) are termed transversions. Alternatively, a nucleic acid may be replaced by a modified nucleic acid as exemplified by replacement of a thymine by thymine glycol. Mutations may result in a mismatch. The term mismatch refers to a non-covalent interaction between two nucleic acids, each nucleic acid residing on a different nucleotide sequence or nucleic acid molecule, which does not follow the base-pairing rules. For example, for the partially complementary sequences 5′-AGT-3′ and 5′-AAT-3′, a G-A mismatch (a transition) is present. A mutation may have a dominant negative effect, i.e., the resulting gene product, even when present in the heterozygous state, can effect certain cellular functions despite the presence of the wild-type copy, for example, in case the product of the dominant negative mutation can still interact with the same elements as the product encoded by the wild-type gene, but block some aspect of its function.

The terms “nucleotide” and “nucleic acid” with reference to a sequence or a molecule are used interchangeably herein and refer to a single-or double-stranded DNA or RNA of natural or synthetic origin. The term nucleotide sequence is thus used for any DNA or RNA sequence independent of its length, so that the term comprises any nucleotide sequence comprising at least one nucleotide, but also any kind of larger oligonucleotide or polynucleotide. The term(s) thus refer to natural and/or synthetic deoxyribonucleic acids (DNA) and/or ribonucleic acid (RNA) sequences, which can optionally comprise synthetic nucleic acid analoga. A nucleic acid according to the present disclosure can optionally be codon optimized. “Codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. As used herein, “nucleotide” can thus generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)).

The term “ortholog” or “orthologues” defines a pair of genes that derives from the same ancestral gene by speciation in the course of evolution. Normally, orthologs retain the same function in the course of evolution.

The term “paralog” or “paralogues” defines a pair of genes that derives from the same ancestral gene by duplication within a genome, wherein the genes in a given cell reside at different locations within the same genome.

The term “particle bombardment” as used herein, also named “biolistic transfection” or “microparticle-mediated gene transfer”, refers to a physical delivery method for transferring a coated microparticle or nanoparticle comprising a nucleic acid or a genetic construct of interest into a target cell or tissue. The micro or nanoparticle functions as projectile and is fired on the target structure of interest under high pressure using a suitable device, often called gene-gun. The transformation via particle bombardment uses a microprojectile of metal covered with the gene of interest, which is then shot onto the target cells using an equipment known as “gene gun” at high velocity fast enough (˜1500 km/h) to penetrate the cell wall of a target tissue, but not harsh enough to cause cell death. For protoplasts, which have their cell wall entirely removed, the conditions are different logically. The precipitated nucleic acid or the genetic construct on the at least one microprojectile is released into the cell after bombardment, and integrated into the genome. The acceleration of microprojectiles is accomplished by a high voltage electrical discharge or compressed gas (helium). Concerning the metal particles used it is mandatory that they are non-toxic, non-reactive, and that they have a lower diameter than the target cell. The most commonly used are gold or tungsten. There is plenty of information publicly available from the manufacturers and providers of gene-guns and associated system concerning their general use.

A “pathogen” as used herein refers to an organism or virus which can infect a plant, or which can cause a disease in a plant, or which can harm a plant. Pathogens showing at least one intracellular or biotrophic phase which can infect a plant, or which can cause a disease in a plant, include fungi, oomycetes, bacteria, viruses, viroids, virus-like organisms, phytoplasmas, protozoa, nematodes and parasitic plants. Plant parasites can cause damage by feeding on a plant and can be selected from ectoparasites like insects, comprising aphids and other sap-sucking insect, mites, and vertebrates. Further included are, for example, necrotrophic fungi secreting toxins and enzymes that kill host cells and then take up nutrients released from the dead cells or tissue.

The term “plant” as used herein is to be construed broadly and refers to a whole plant organism, a plant organ, differentiated and undifferentiated plant tissues, plant cells, seeds, and derivatives and progeny thereof. “Plant cells” include without limitation, for example, cells from seeds, from mature and immature embryos, meristematic tissues, seedlings, callus tissues in different differentiation states, leaves, flowers, roots, shoots, gametophytes, grains, kernels, sporophytes, pollen and microspores, protoplasts, macroalgae and microalgae. The different plant cells can either be haploid, diploid or multiploid. The term “plant organ” refers to plant tissue or a group of tissues that constitute a morphologically and functionally distinct part of a plant. Typically, the term “grain” is used to describe the mature kernel produced by a plant grower for purposes other than growing or reproducing the species, and “seed” means the mature kernel used for growing or reproducing the species. For the purposes of the present invention, “grain”, “seed”, and “kernel”, will be used interchangeably.

A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

“Progeny” comprises any subsequent generation of a plant, plant cell, plant tissue, or plant organ.

The terms “protein”, “amino acid” or “polypeptide” are used interchangeably herein and refer to an amino acid sequence having a catalytic enzymatic function or a structural or a functional effect. The term “amino acid” or “amino acid sequence” or “amino acid molecule” comprises any natural or chemically synthesized protein, peptide, polypeptide and enzyme or a modified protein, peptide, polypeptide and enzyme, wherein the term “modified” comprises any chemical or enzymatic modification of the protein, peptide, polypeptide and enzyme, including truncations of a wild-type sequence to a shorter, yet still active portion. In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D.N. Glover ed. 1985); Oligonucleotide Synthesis (M.J. Gait ed. 1984); Nucleic Acid Hybridization (B.D. Hames & SJ. Higgins eds. (1985); Transcription and Translation (B.D.

Hames & S.J. Higgins, eds. (1984); Animal Cell Culture (RI. Freshney, ed. (1986); Immobilized Cells and Enzymes (IRL Press, (1986); B. Perbal, A Practical Guide To Molecular Cloning(1984); F.M. Ausubel et al. (eds.), Current Protocols in Molecular Biology, John Wiley & Sons, Inc. (1994); among others.

The term “regulatory region” as used herein refers to a nucleic acid sequence, which can direct and/or influence the transcription and/or translation and/or modification of a nucleic acid sequence of interest. A regulatory sequence may comprise at least one of a core promoter sequence, a proximal promoter sequence, a cis regulatory sequence, a trans regulatory sequence, a locus control sequence, an insulator sequence, a silencer sequence, an enhancer sequence, a terminator sequence, and/or any combination thereof. In addition to a nucleic acid sequence “regulatory sequence” or “regulatory region”, there may be “regulatory domains” or “regulatory proteins/enzymes”. These amino acid sequences regulate, for example, transcription, RNA capping, splicing, polyadenylation, chromatin structure, signaling events, post-translational modifications and the like by (i) binding to, or (ii) by catalyzing a relevant reaction, e.g., by (i) blocking signaling by binding to a signaling domain, or regulating transcription as transcription factor acting on a nucleic acid sequence in trans, or (ii) by (de) phosphorylation events, e.g., as it is the case for many kinases.

The terms “site-directed/specific DNA modifying enzyme”, “site-specific effector”, or “site-specific nuclease” are used interchangeably herein and refer to a protein or a functional fragment thereof which is able to introduced a modification such as a double-stranded DNA break (DSB) or single-strand DNA break at a target site of a genomic sequence in a site-specific manner, either alone, or in combination with further molecules in a molecular complex. A “base editor” or “base editor complex” comprises at least one site-directed/specific DNA modifying enzyme which is able to induce a targeted base exchange at a target site of a genomic sequence.

The term “TILLING” as used herein is an abbreviation for “Targeting Induced Local Lesions in Genomes” and describes a well-known reverse genetics technique originally designed to detect unknown SNPs (single nucleotide polymorphisms) in genes of interest using an enzymatic digestion and is widely employed in plant and animal genomics. The technique allows for the high-throughput identification of an allelic series of mutants with a range of modified functions for a particular gene. TILLING combines mutagenesis (e.g., chemical or via UV-light) with a sensitive DNA screening-technique that identifies single base mutations.

The terms “transgene” or “transgenic” as used herein refer to at least one nucleic acid sequence that is taken from the genome of one organism, or produced synthetically, and which is then introduced into a host cell or organism or tissue of interest and which is subsequently integrated into the host' s genome by means of “stable” transformation or transfection approaches. In contrast, the term “transient” transformation or transfection or introduction refers to a way of introducing molecular tools including at least one nucleic acid (comprising at least one of DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell.

The term “transient introduction” as used herein thus refers to the transient introduction of at least one nucleic acid and/or amino acid sequence according to the present disclosure, preferably incorporated into a delivery vector or into a recombinant construct, with or without the help of a delivery vector, into a target structure, for example, a plant cell, wherein the at least one nucleic acid sequence is introduced under suitable reaction conditions so that no integration of the at least one nucleic acid sequence into the endogenous nucleic acid material of a target structure, the genome as a whole, occurs, so that the at least one nucleic acid sequence will not be integrated into the endogenous DNA of the target cell. As a consequence, in the case of transient introduction, the introduced genetic construct will not be inherited to a progeny of the target structure, for example a prokaryotic, an animal or a plant cell. The at least one nucleic acid and/or amino acid sequence or the products resulting from transcription, translation, processing, post-translational modifications or complex building thereof are only present temporarily, i.e., in a transient way, in constitutive or inducible form, and thus can only be active in the target cell for exerting their effect for a limited time. Therefore, the at least one sequence or effector introduced via transient introduction will not be heritable to the progeny of a cell. The effect mediated by at least one sequence or effector introduced in a transient way can, however, potentially be inherited to the progeny of the target cell.

A “variant” in the context of a nucleic acid or amino acid sequence protein means a nucleic acid or amino acid sequence derived from the native nucleic acid or amino acid sequence, or another starting sequence, by deletion (so-called truncation) or addition of one or more sequences to the 5′/N-terminal and/or 3′/C-terminal end of the native nucleic acid or amino acid sequence; deletion or addition of one or more nucleic acid or amino acid sequence at one or more sites in the native nucleic acid or amino acid sequence; or substitution of one or more nucleic acid or amino acid sequence at one or more sites in the native protein. Variant proteins encompassed by the present invention are biologically active, that is they continue to possess all or some of the activity of the native proteins of the invention as described herein. Such variants may result from, for example, genetic polymorphism or from human manipulation.

Whenever the present disclosure relates to the percentage of the homology or identity of nucleic acid or amino acid sequences these values define those as obtained by using the EMBOSS Water Pairwise Sequence Alignments (nucleotide) programme (www.ebi.ac.uk/Tools/psa/emboss_water/nucleotide) nucleic acids or the EMBOSS Water Pairwise Sequence Alignments (protein) programme (www.ebi.ac.uk/Tools/psa/emboss_water/) for amino acid sequences, preferably over the entire length of the sequence, i.e., any percentage value provided means the % homology or % identity as measured over the whole length of a subject or starting sequence in comparison to an identical or variant further sequence. Those tools provided by the European Molecular Biology Laboratory (EMBL) European Bioinformatics Institute (EBI) for local sequence alignments use a modified Smith-Waterman algorithm (see www.ebi.ac.uk/Tools/psa/and Smith, T.F. & Waterman, M.S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5. Furthermore, bioinformatics tools for multiple sequence alignments for nucleic acid and amino acid sequences are readily available to the skilled person and can, for example, be obtained from EMBL/EBI, including Clustal Omega, Kalign, MAFFT, MUSCLE, MView, T-Coffee, or WebPRANK.

### BRIEF DESCRIPTION OF THE DRAWINGS

FIG. 1 shows identities between the different protein sequences (SEQ ID NOs: 11-14, 16-17, 15 and 18-19) of the CPL3 genes and comparison to the Arabidopsis AtCPL3 protein sequence (reference sequence: SEQ ID NO: 20). FIG. 1A: Sequence Alignments of amino acid sequences encoded by the CPL3 genes of six different crops compared to the Arabidopsis thaliana reference sequence; FIG. 1B: shows percent identities determined from the alignment. As evident when performing a sequence alignment of the respective protein sequences, said sequences significantly vary in the different plants (Arabidopsis thaliana, Glycine max, Solanum tuberosum, Triticum aestivum, Sorghum bicolor, Beta vulgaris and Zea mays, respectively), i.e., sequence identities of between 36% and 45% in comparison to the Arabidopsis reference sequence were observed.

FIG. 2 shows identities between the different coding sequences of the CPL3 genes (SEQ ID NOs: 2-3, 10, 4, 6-7, 5 and 8-9) in comparison to the Arabidopsis AtCPL3 coding sequence as reference sequence (reference sequence: SEQ ID NO: 1). FIG. 2A: Sequence Alignments of coding sequences of the CPL3 genes of six different crops compared to the Arabidopsis thaliana reference sequence; FIG. 2B: shows percent identities determined from the alignment. As evident when performing a sequence alignment of the respective gene sequences, said sequences significantly vary in the different plants (Arabidopsis thaliana, Glycine max, Solanum tuberosum, Triticum aestivum, Sorghum bicolor, Beta vulgaris and Zea mays, respectively), i.e., sequence identities of between 44% and 53% in comparison to the Arabidopsis reference sequence were observed.

FIG. 3 shows the results of TaCPL3-virus induced gene silencing (VIGS) experiment further detailed below in Example 1. VIGS of all TaCPL3 homeologues by Barley Stripe Mosaic Virus (BSMV) with the silencing sequence TaCPL3_fragA also called TaCPL3-A (SEQ ID NO: 5 and 15) or TaCPL2_fragB also called TaCPL3-B (SEQ ID NO: 6 and 16) resulted in reduced pycnidia and spore formation of Zyoseptoria tritici infected wheat leaved from cultivar Taifun. Pycnidia: n=10 plants with 2 analyzed leaves of each plant. Spores: spores of 5 leaves were washed and counted (n=8). ¹: numbers with different capitals are statistically different according to ANOVA.

FIG. 4 shows a vector map of the plasmid construct used for maize transformation to silence ZmCPL3 (Example 2 and SEQ ID NO: 24).

FIG. 5 shows the results of Setosphaeria turcica resistance assay: FIG. 5A: assay with segregating T1 plants of ZmCPL3_RNAi transformation events MTR0374-T-038 and MTR0374-T-053 as further illustrated in Example 2. ¹: ntg=non-transgenic segregant (azygous line). ²: Fungal biomass was determined by quantitative PCR on DNA extracted from infected leaves after the resistance assay. Quantitative PCR with maize DNA-specific primers was used for data normalization. ³: Gene expression for the different lines was determined by ZmCPL3-specific quantitative reverse transcription PCR. Data is normalized to the expression of the housekeeping gene ZmEF1. Data was generated with plants that were not infected; FIG. 5B: assay with T2 lines and the respective azygous sisterlines (null segregants) as further illustrated in Example 2. ¹: number with different capitals are statistically according to ANOVA. ²: ntg=non-transgenic segregant (azygous line). ³: measured before plant inoculation.

FIG. 6 shows a representative picture of the results of infected leaves for Setosphaeria turcica resistance assay with homozygous T2 lines and the respective azygous sisterlines (null segregants). ZmCPL3_RNAi experiments (T2 plants) as further illustrated in Example 2.

FIG. 7 shows enhanced Fusarium resistance of Taifun after VIGS mediated silencing of CPL3 (see also Table 8).

FIG. 8 shows wheat plant heads after Fusarium graminearum infection. a. transformed with empty vector, b. untreated, c. silencing control, d. VIGS-silenced CPL3.

FIG. 9 shows MAD7 nuclease activity at multiple Zm-CPL3 target sites in maize protoplasts. Maize line A188 protoplasts were independently co-transfected with constructs that express the MAD7 nuclease protein gene and one of a variety of crRNA sequences to specific sites in the gene. Although target sites were pre-selected based on the same criteria, there were differences observed in the INDEL frequencies. Stippled bars show the raw data from protoplast treatments while the striped set are based on extrapolated values based on 100% protoplast transfection.

FIG. 10 shows graphical sequence representation for Zm-CPL3 exon 1 showing the positions of tested target sites. CPL3 exon 1 is shown in yellow and the crRNA sequences are labelled in grey to demonstrate the position of potential double stranded breaks from each target sequence or construct. In addition, the green markers are indicating the primers used for PCR to generate the amplicon for Sanger sequence analysis and DRIVE. Figure discloses SEQ ID NO: 48.

### DETAILED DESCRIPTION

The present invention is based on the identification of CPL3 homologues in a large variety of different plant species, including major crop plants, which were functionally characterized and which were shown to be crucial for plant immunity leading to increased pathogen resistance, importantly also towards hemibiotrophic fungal pathogens. The findings of the present invention indicate that the desired effect of pathogen resistance can be achieved by a targeted modulation of the CPL3 gene making a complete knock out unnecessary, which knock-out might be associated with undesired effects like impaired plant growth or undesired signaling functions due to the lack of an CPL effector. The findings of the present invention indicate that the desired effect of pathogen resistance can be achieved by a simple knock-down of the CPL3 gene, or by introducing a targeted mutation into a CPL3 gene, or a regulatory sequence thereof, including also combinations of these strategies, making a complete knock-out unnecessary as modulation of the CPL3 function is mediated based on the understanding of the functional interplay of CPL3 with other effectors in plant immunity to achieve pathogen resistance, preferably also resistance against hemibiotrophic pathogens, which are known for their complex lifestyles associated with severe problems in causing plant diseases leading to crop losses. Furthermore, downregulation of CPL3 genes by silencing constructs, or as achieved by RNA editing, or by creating and providing dominant negative mutant alleles avoids the negative effect on plant growth reported by e.g., Koiwa et al. (2002) and potential further side effects associated with a manipulation of a central molecule in plant immunity. Finally, CPL3 downregulation can thus also be achieved by the introduction of a dominant-negative allele of CPL3, for example, by the introduction of a targeted point mutation which leads to dominant CPL3-based resistance.

In a first aspect, a plant having pathogen resistance, wherein pathogen resistance is conferred or increased by modulation of a nucleotide sequence encoding an endogenous C-terminal domain phosphatase-like 3 (CPL3) protein or a regulatory sequence thereof, or by modulation of the transcription of an endogenous CPL3 protein, wherein modulation is achieved by (i) one or more mutation(s) of the nucleotide sequence encoding a CPL3 protein, preferably wherein the one or more mutation(s) has/have a dominant negative effect, preferably wherein the one or more mutation(s) cause(s) an alteration of the amino acid sequence of the conserved catalytic domain of the CPL3 protein comprising the DXDXT/V motif; and/or (ii) one or more silencing construct(s) directed to one or more endogenous nucleotide sequence(s) encoding a CPL3 protein, preferably directed to all endogenous nucleotide sequences encoding a CPL3 protein; and/or (iii) a modification of the native regulatory sequence(s) of one or more nucleotide sequence(s) encoding an endogenous CPL3 protein, preferably all nucleotide sequences encoding an endogenous CPL3 protein, wherein the modification causes a reduced expression rate of the one or more nucleotide sequence(s) encoding an endogenous CPL3 protein may be provided. The above aspect thus covers three different modes (i) to (iii) for a targeted modulation, which may be used alone or in combination to obtain a pathogen resistant plant.

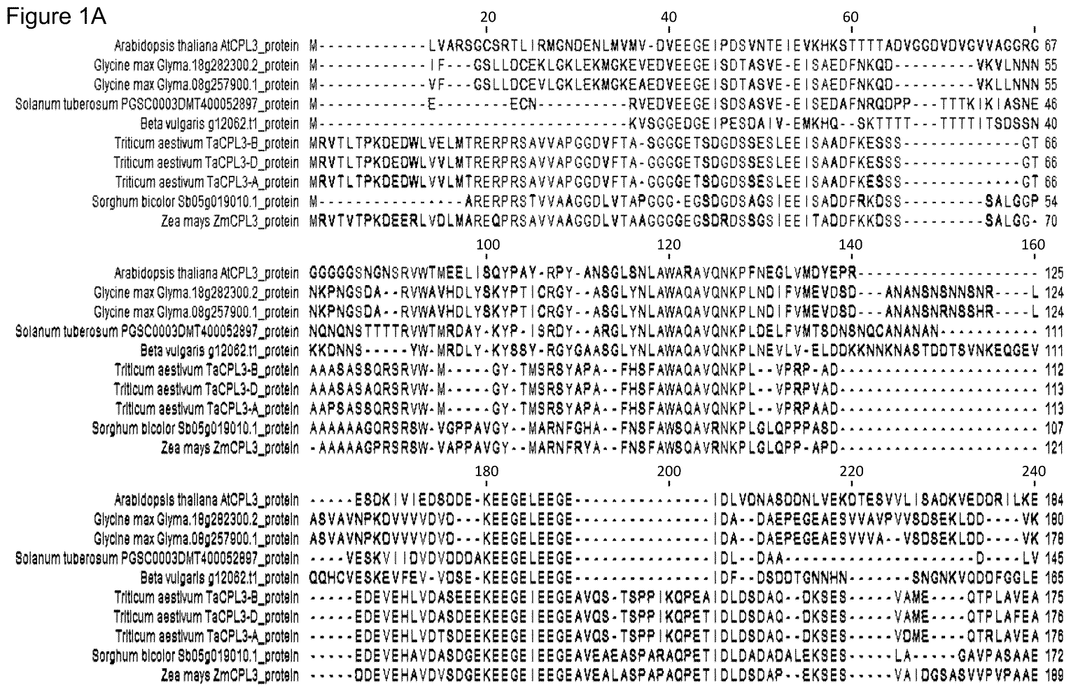

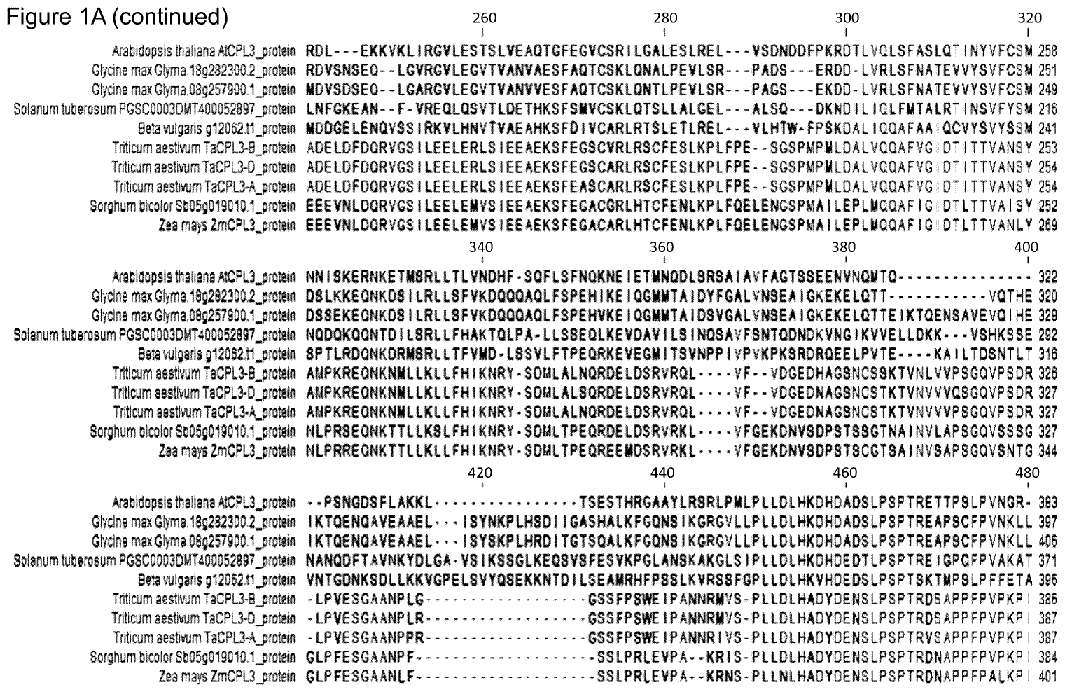

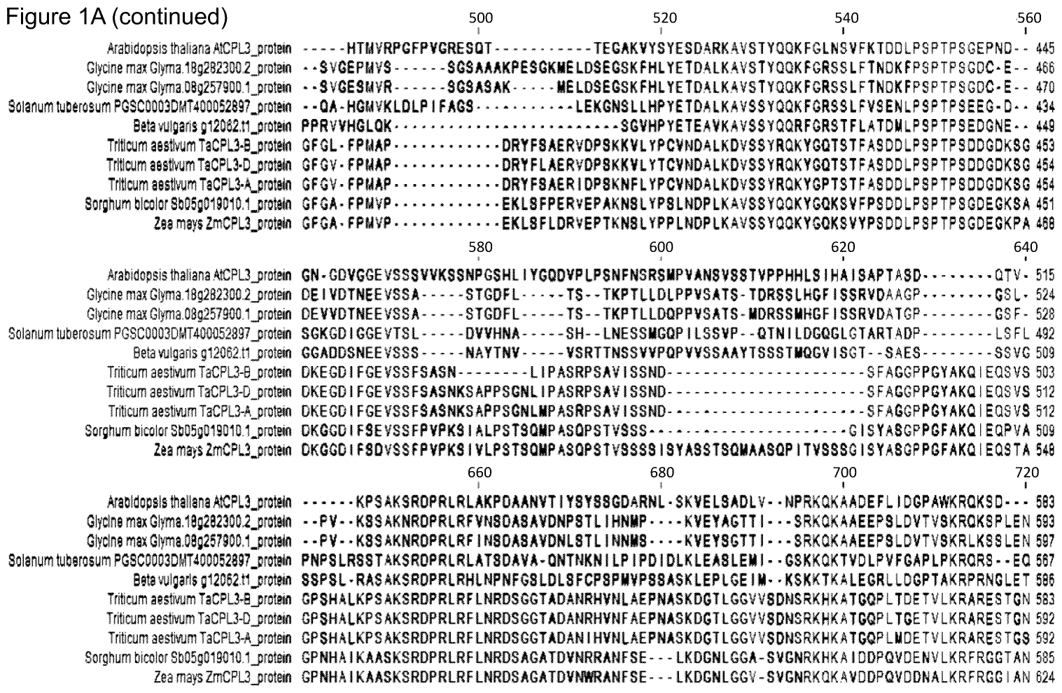

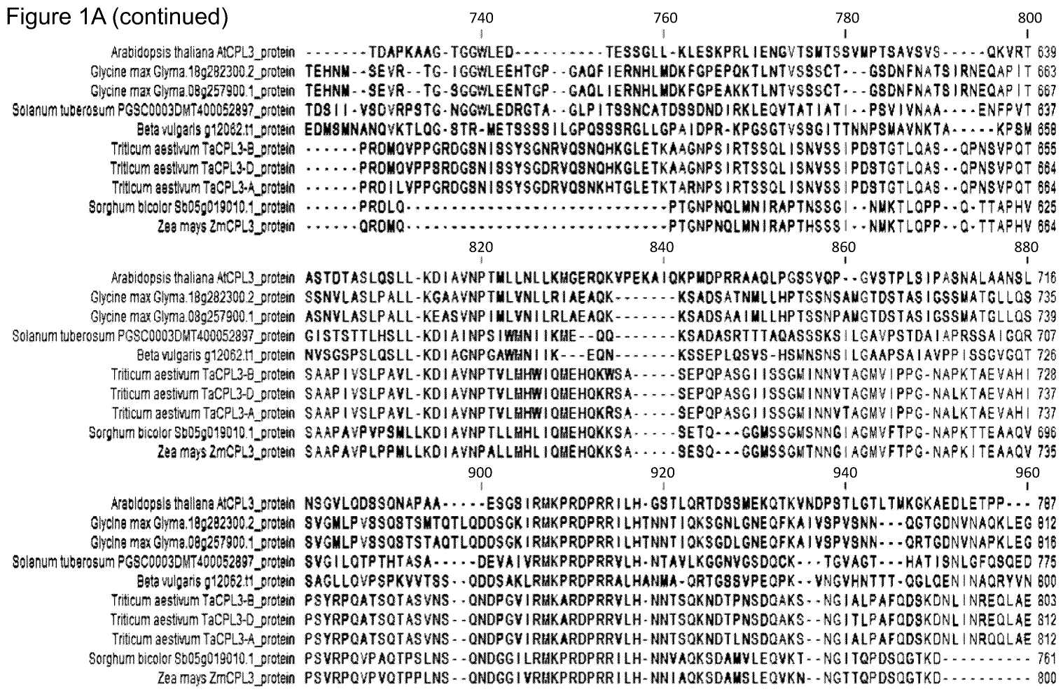

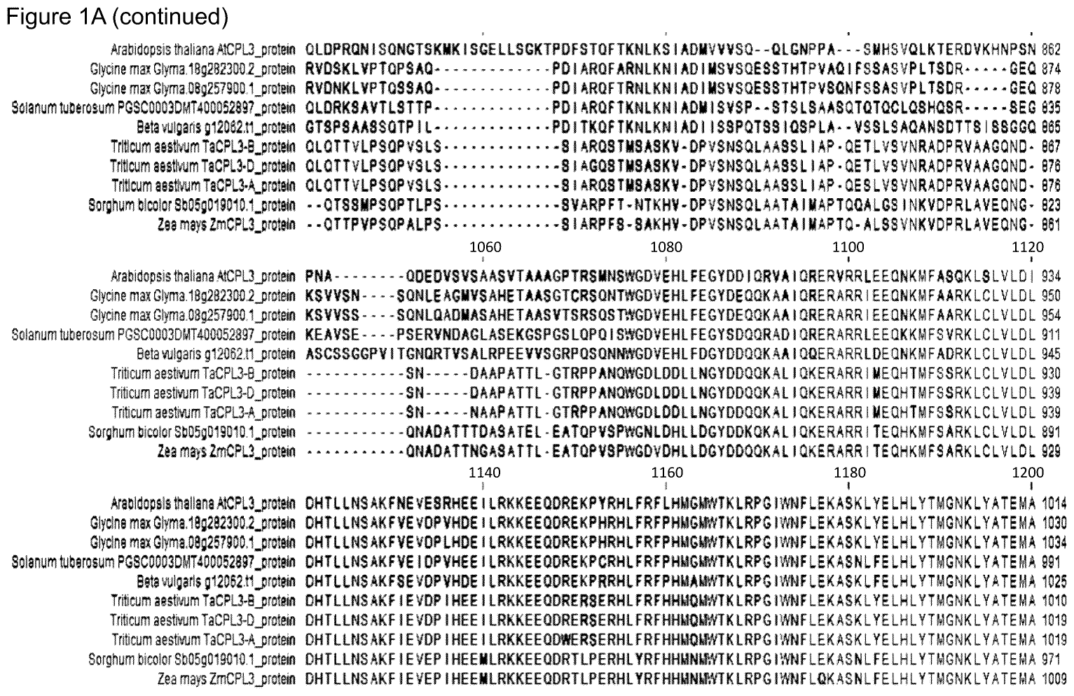

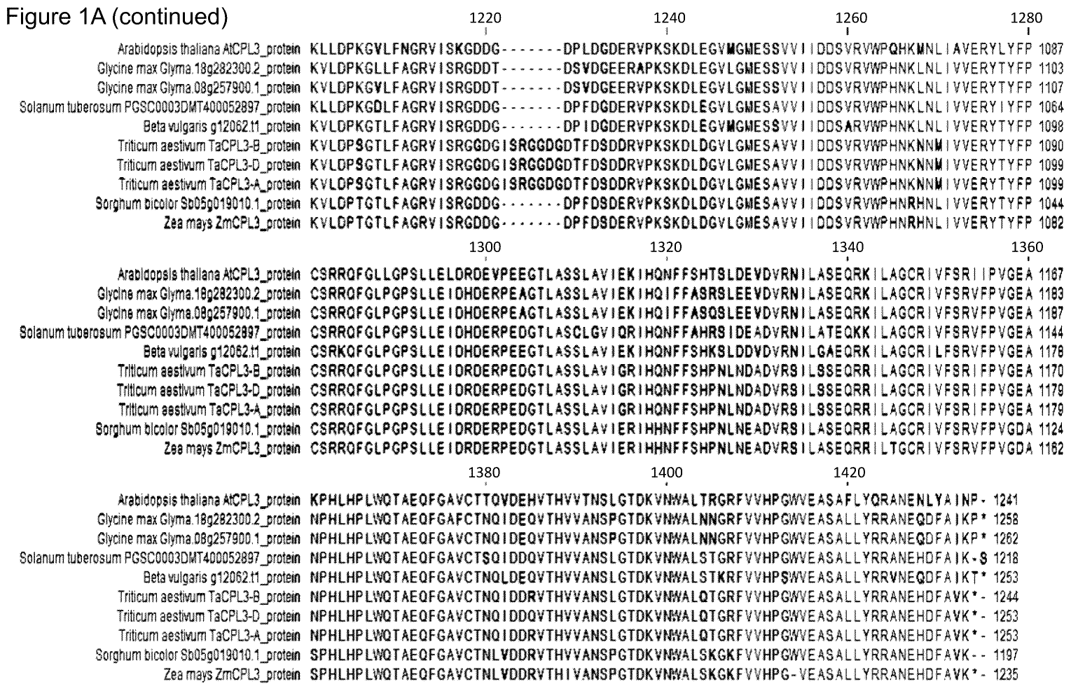

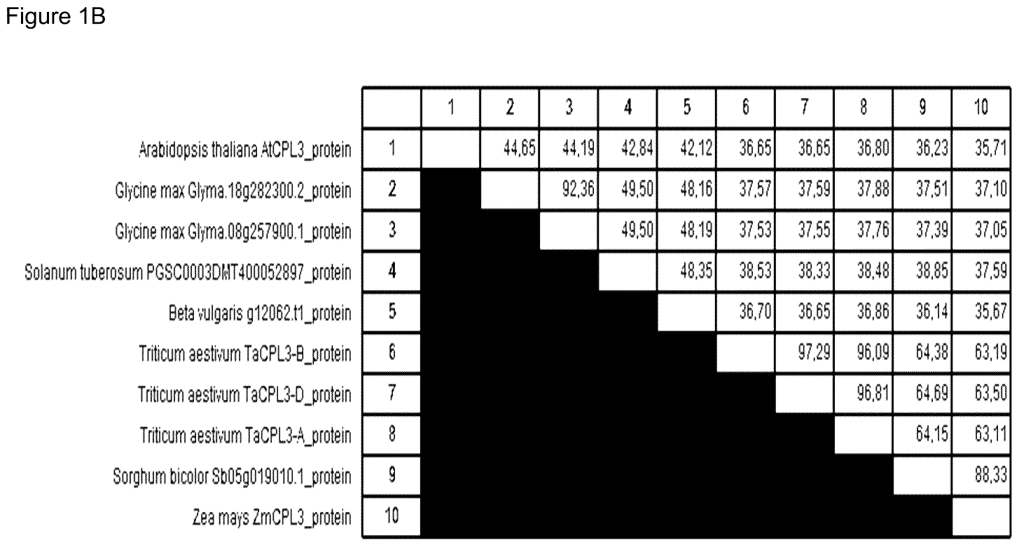

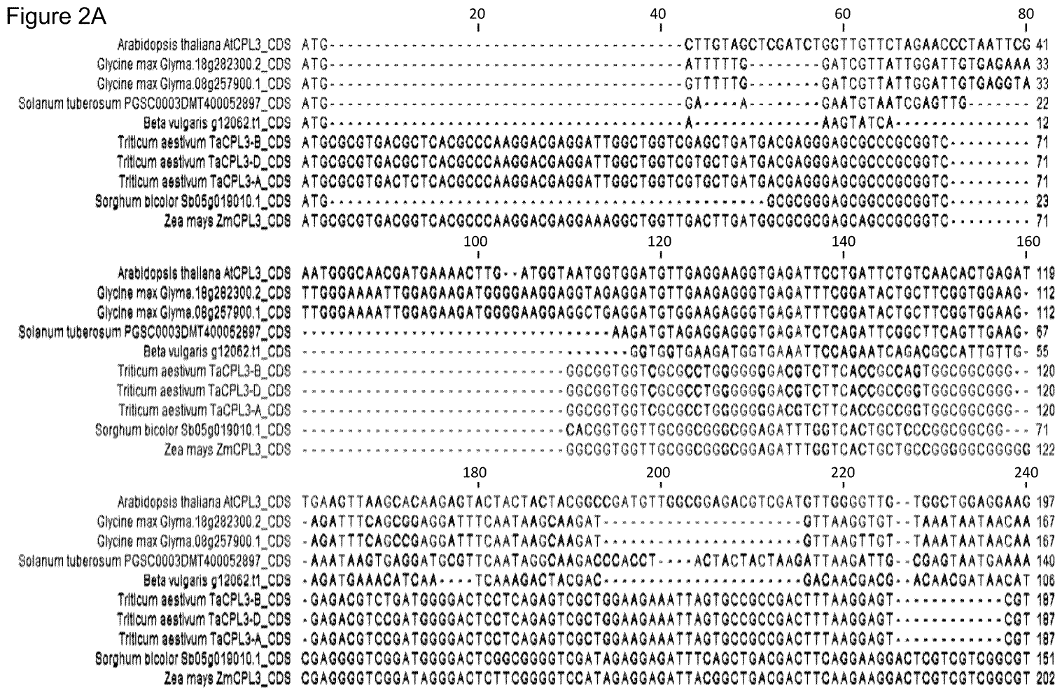

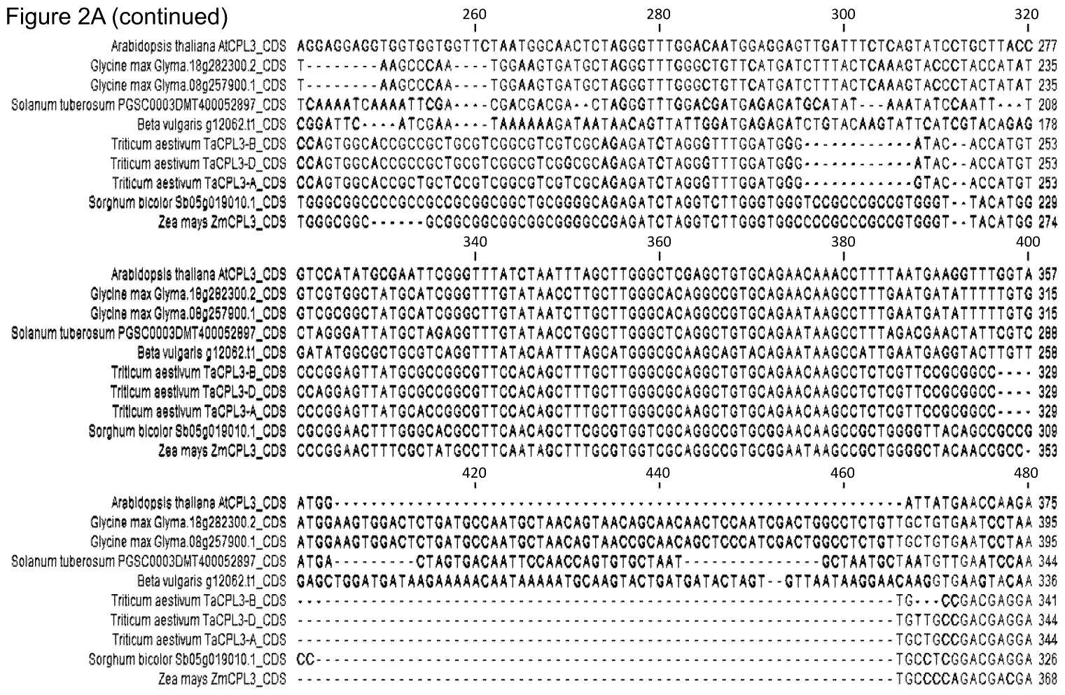

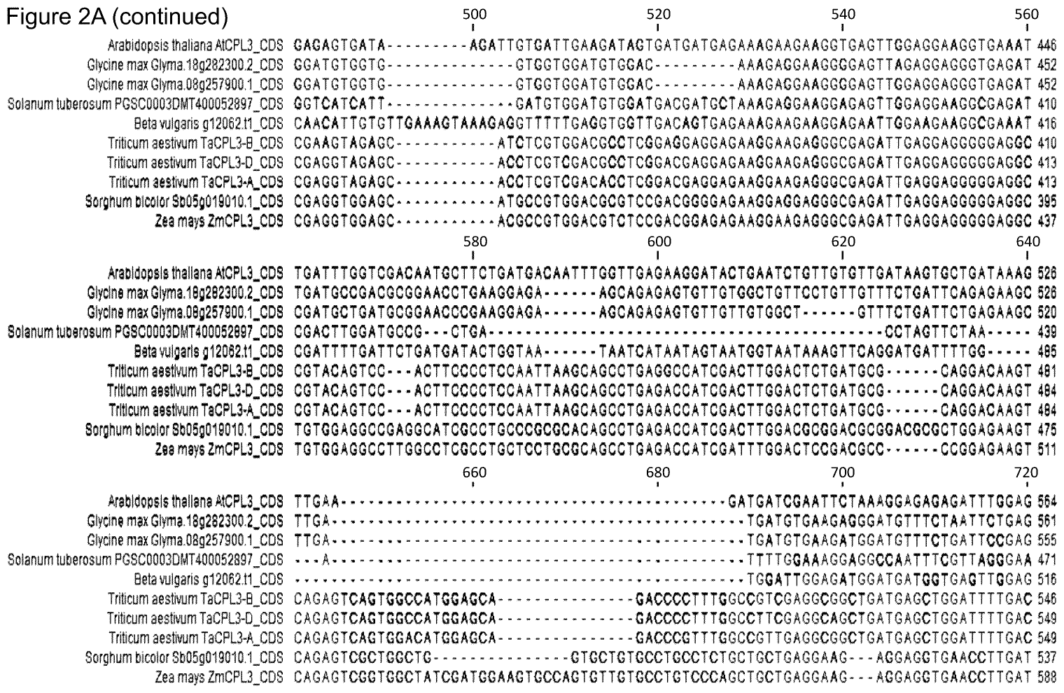

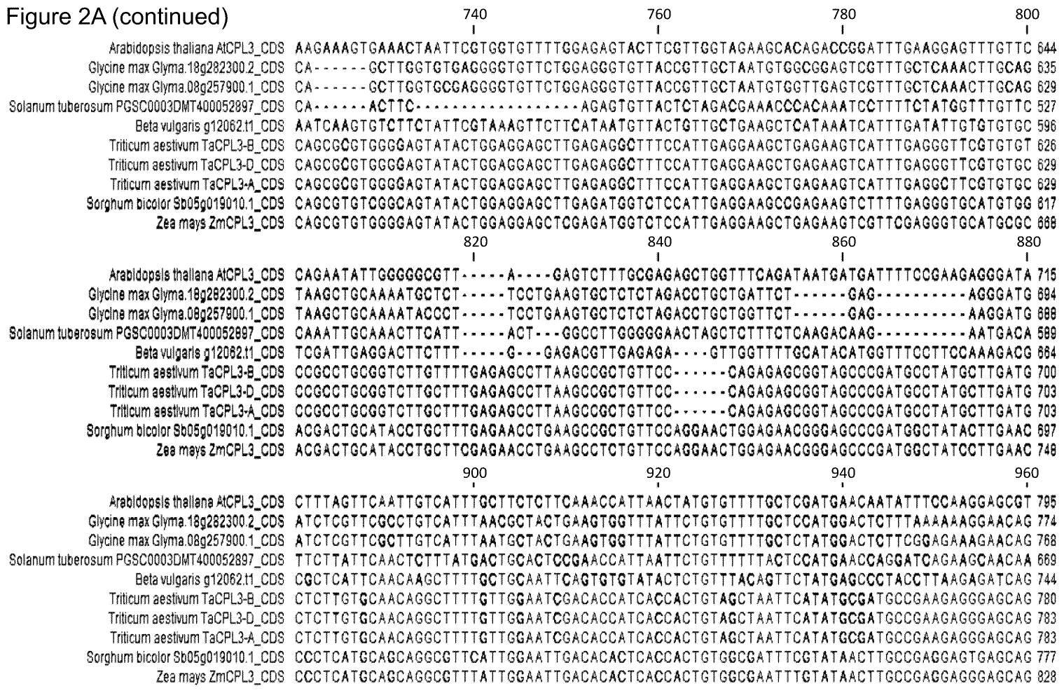

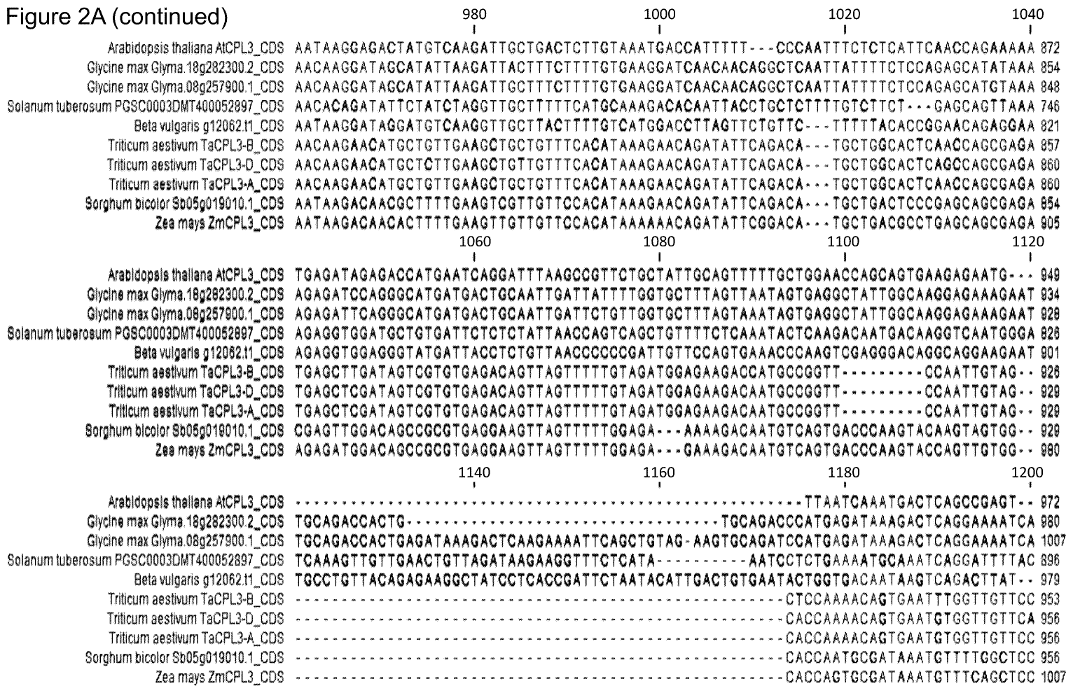

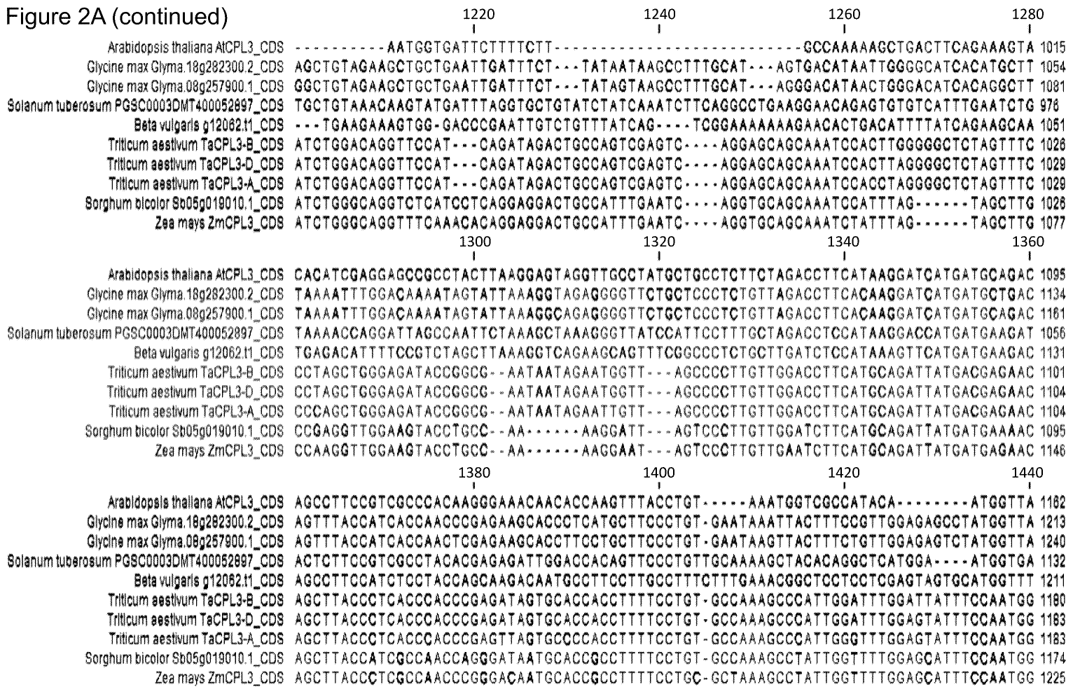

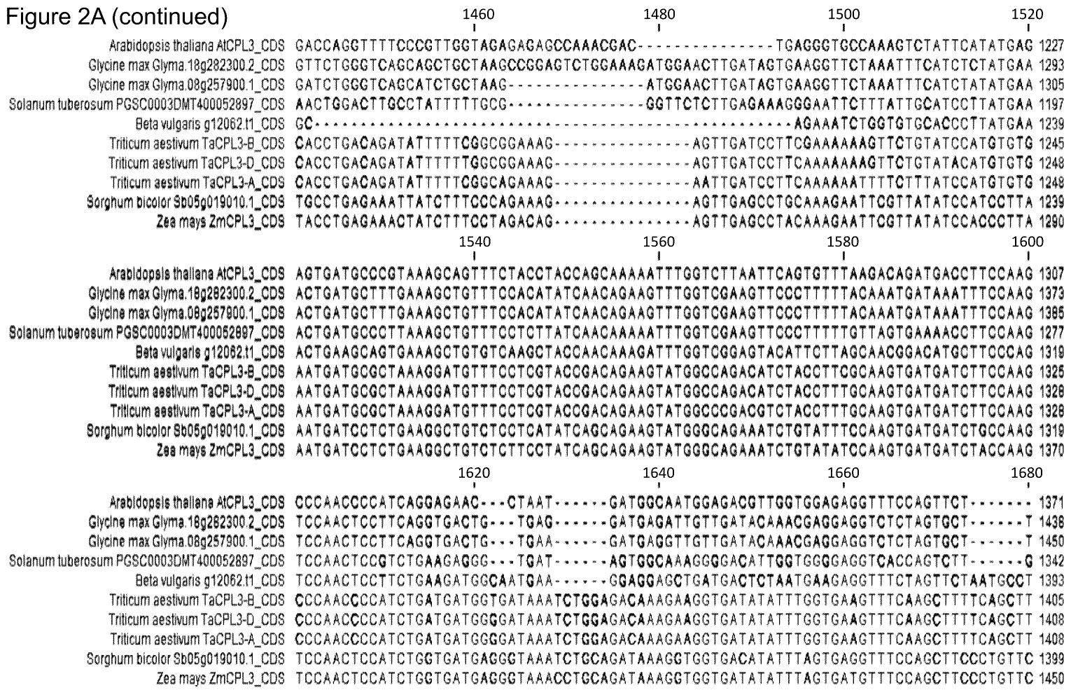

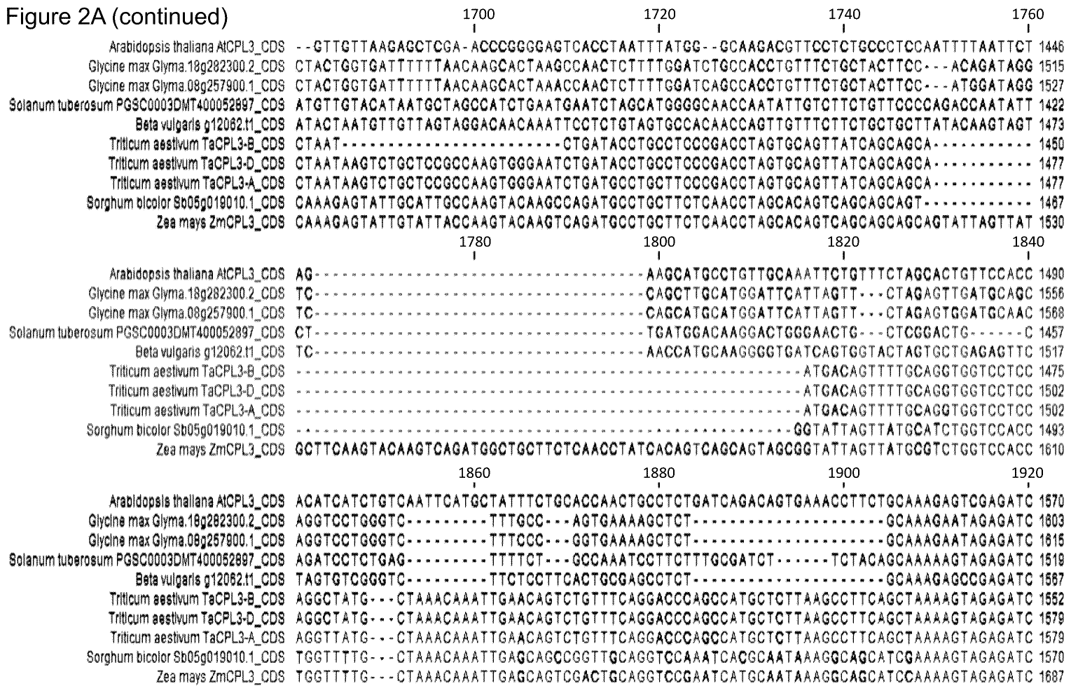

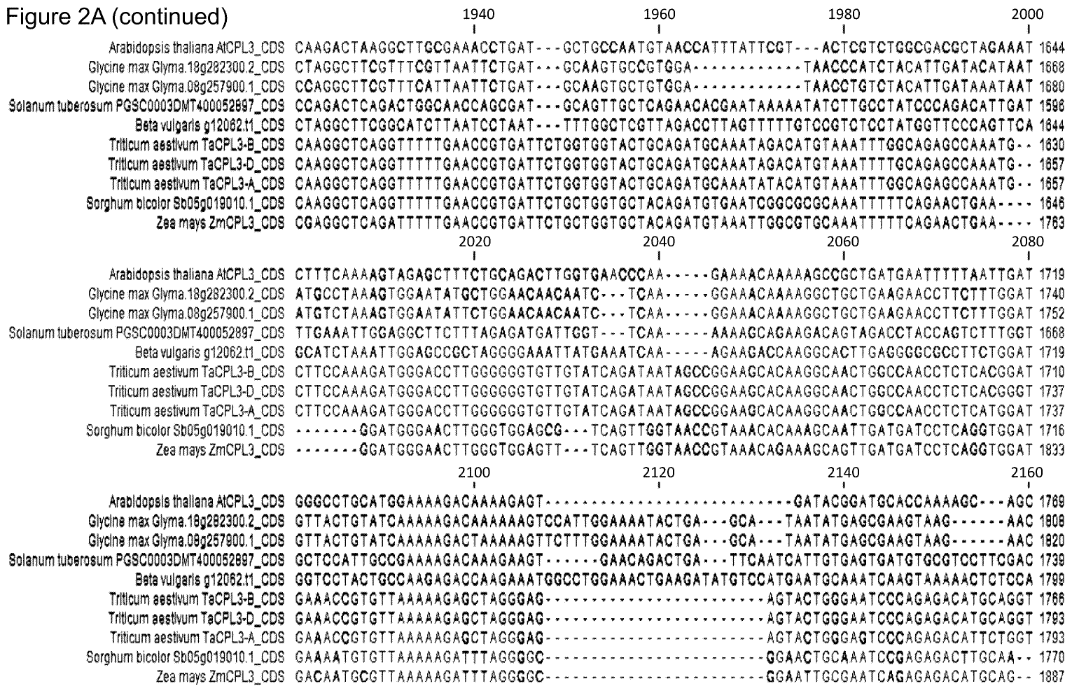

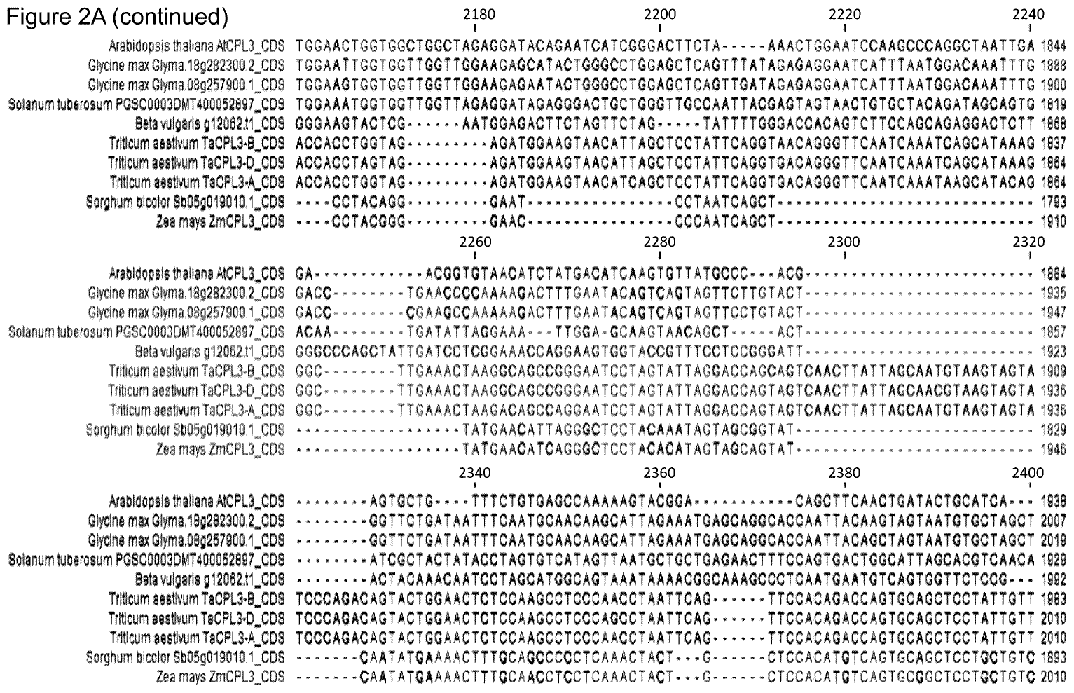

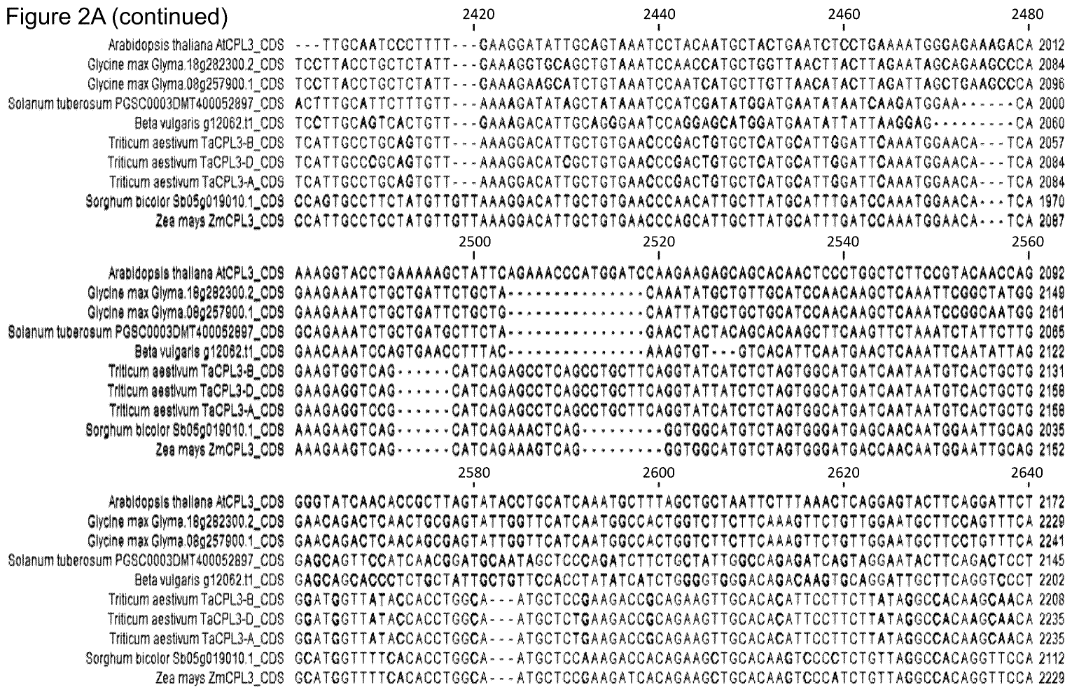

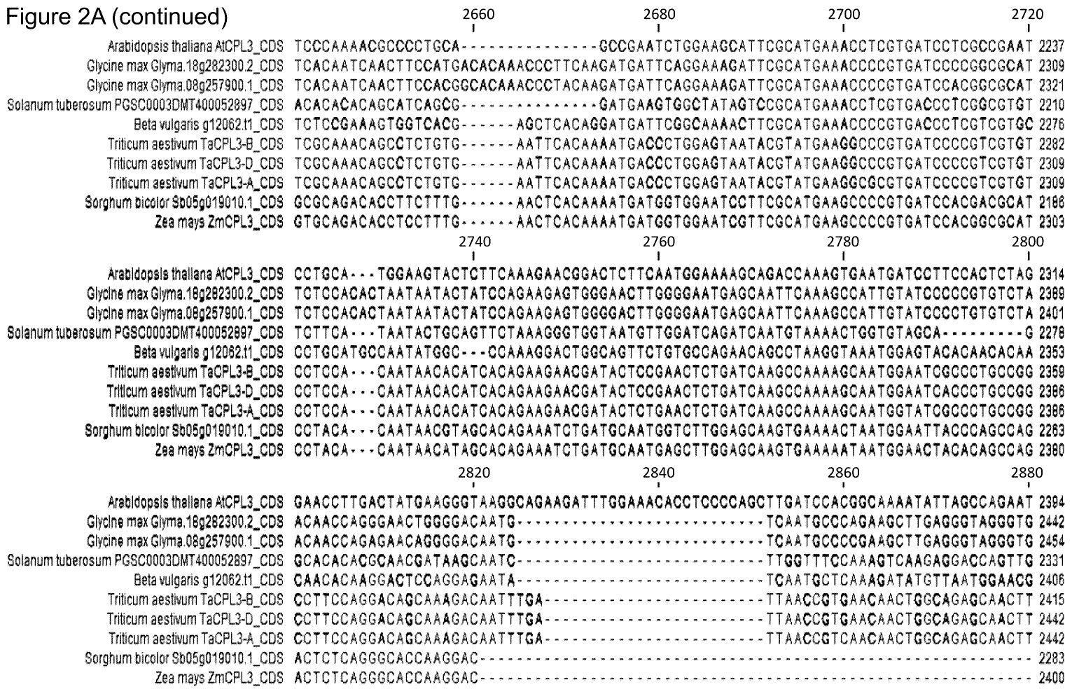

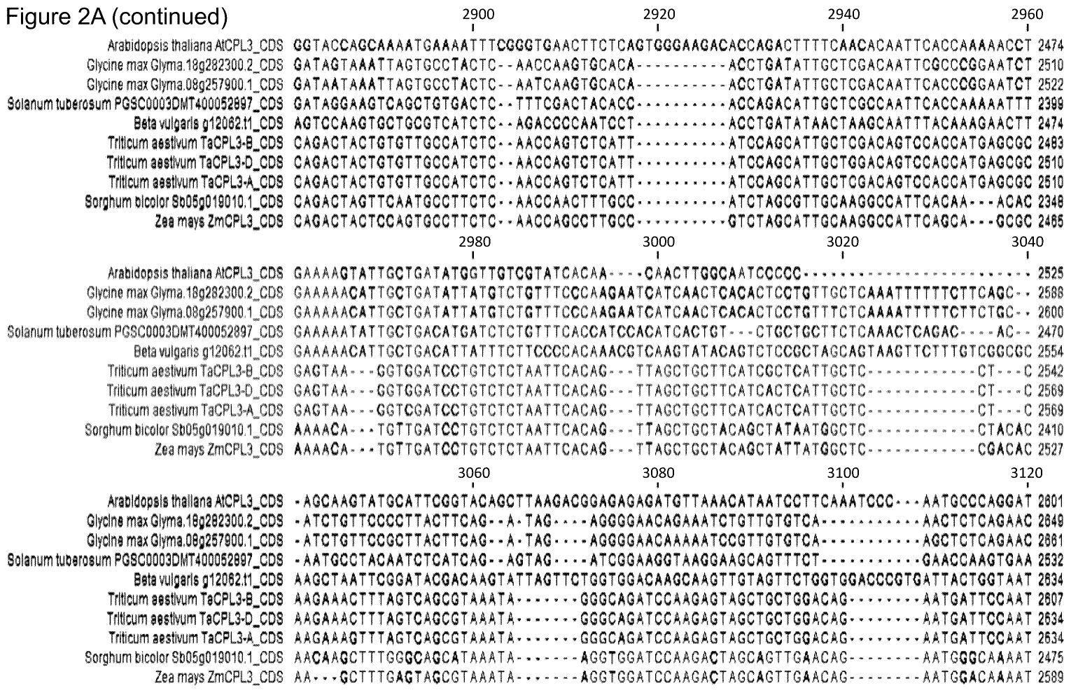

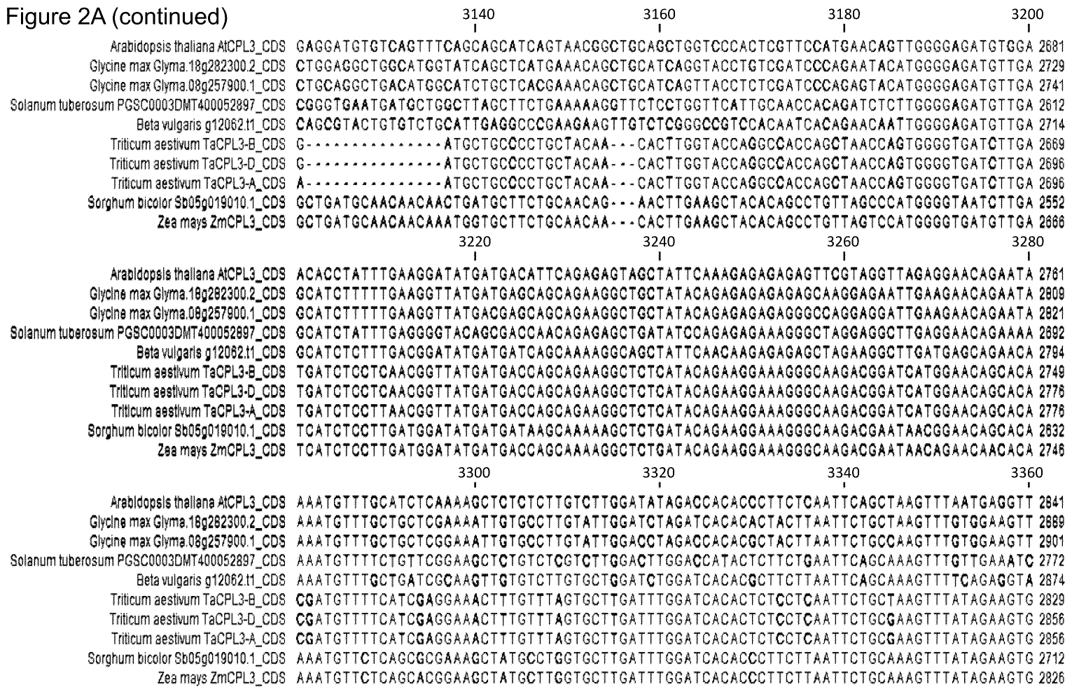

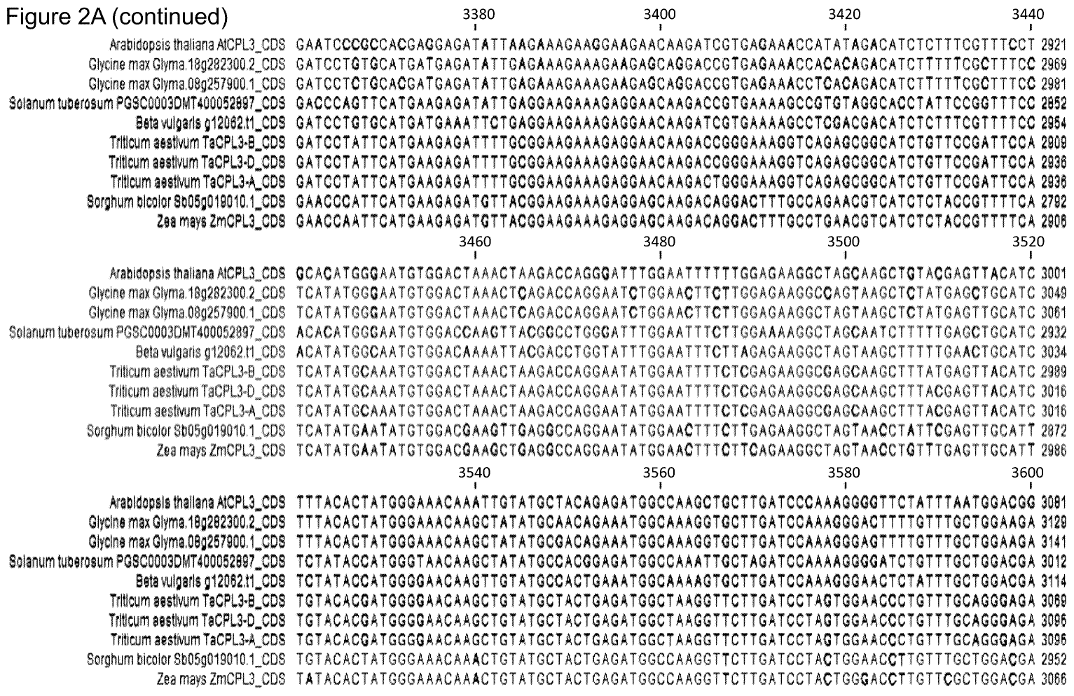

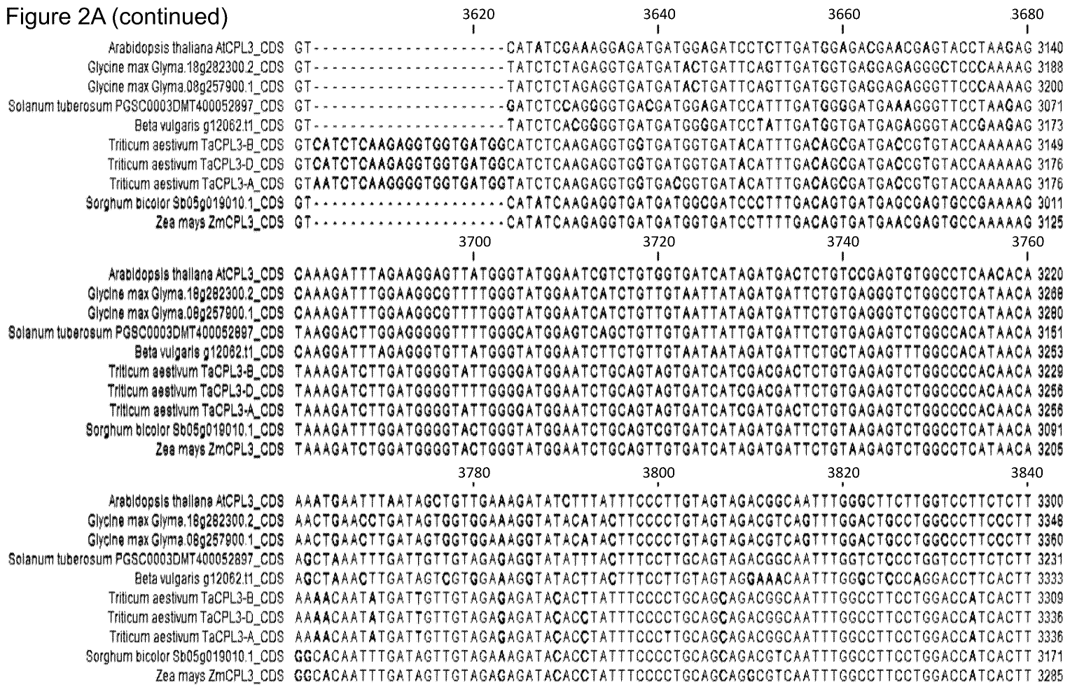

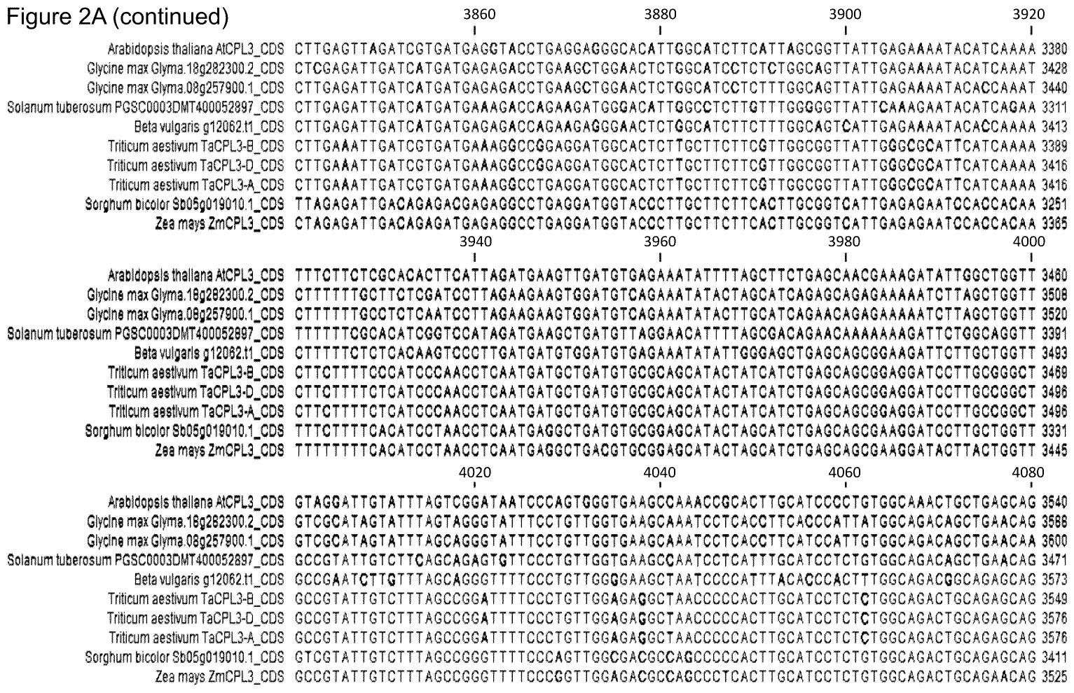

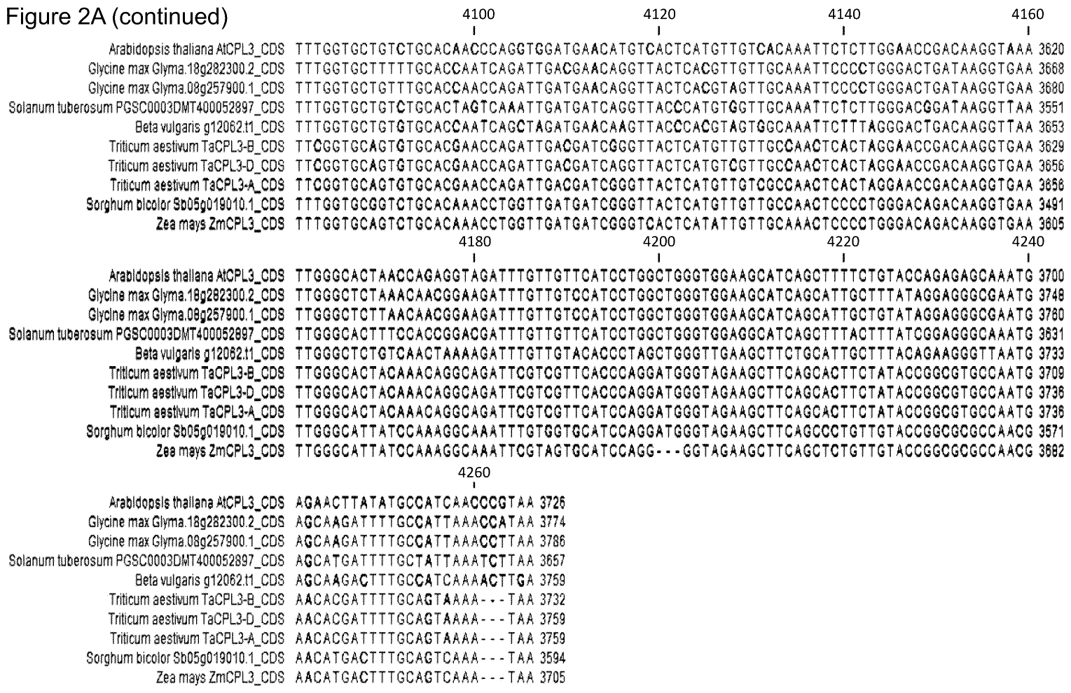

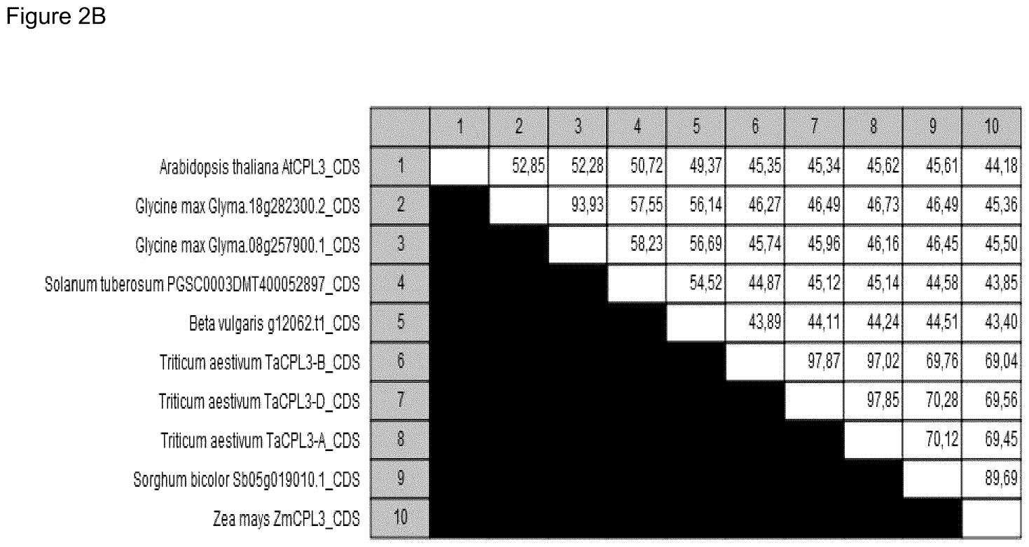

Using homology searches based on an Arabidopsis model gene sequence characterized for plant immunity only as negative regulator of BABA-induced gene expression (Koiwa et al., 2002) so far and in a complete knock-out scenario (SEQ ID NO: 1), several new CPL3 coding genes (SEQ ID NO: 2-10) in multiple crop plants not specifically associated with plant immunity at date were identified, characterized and modulated in a targeted way. As shown in FIGS. 1A-1B and 2A-2B, the identities to of the discovered CPL3 proteins (SEQ ID NO: 11-19) to the Arabidopsis AtCPL3 protein (SEQ ID NO: 20) are between 36% and 45% at amino acid level. Likewise, the identities of the coding sequence (CDS) of the discovered CPL3 genes (SEQ ID NO: 2-10) to the Arabidopsis coding sequence (SEQ ID NO: 1) range from 44% to 53% at the nucleotide level. Furthermore, it was discovered that soybean (Glycine max) contains two paralogous CPL3 sequences. Based on this degree of relationship, it was not obvious at first glance whether the identified genes would have favorable functional features so that a deeper mechanistic analysis and mutational and knock-down studies were necessary.

Surprisingly, it was identified that modulation of at least one, preferably all, new CPL3 alleles, or a regulatory sequence thereof is correlated with increased pathogen resistance in a plant carrying the respective CPL3 alleles as endogenous genes/alleles. Several strategies could thus be identified to modify the DNA or RNA sequence of a CPL3 gene, or also the regulatory sequence like a promoter, alone or in combination, which turned out to be superior to creating a full knock-out of a CPL3 gene, probably due to the relevant function CPL gene products fulfill in their natural context in plant immunity.

In one embodiment, the plant having pathogen resistance may comprise a nucleotide sequence, wherein the nucleotide sequence encodes a CPL3 protein modified in a targeted way to optimize pathogen resistance, wherein the CPL3 sequence may be selected from the group consisting of: (a) a nucleotide sequence set forth in SEQ ID NOs: 2-10 or a homologous, orthologous or paralogous sequence thereof; (b) a nucleotide sequence having at least 80%, at least 85%, at least 90%, at least 92%, at least 94%, at least 95%, at least 96%, at least 97%, at least 98% or at least 99% identity to a nucleotide sequence as defined in (a); (c) a nucleotide sequence encoding for an amino acid sequence set forth in SEQ ID NOs: 11-19 or for an amino acid sequence which have at least 80%, at least 85%, at least 90%, at least 92%, at least 94%, at least 95%, at least 96%, at least 97%, at least 98% or at least 99% identity to the one sequence as set forth in SEQ ID NOs: 11-19; (d) a nucleotide sequence encoding for an amino acid sequence having at least 80%, at least 85%, at least 90%, at least 92%, at least 94%, at least 95%, at least 96%, at least 97%, at least 98% or at least 99% identity to one of the sequences set forth in SEQ ID NOs: 11-19, or (e) a nucleotide sequence hybridizing with a nucleotide sequence complementary to the nucleotide sequence as defined in (a)-(d) under stringent conditions.

In one embodiment, the pathogen against which an increased resistance of a plant is desired may be at least one of a fungal pathogen, an oomycete pathogen, a bacterial pathogen, a virus, a nematode pathogen, or an insect. Although weeds are the major cause of crop loss on a global scale, major losses are suffered by agricultural crops due to insect damage feeding on the plant as pathogen and other plant diseases caused by various plant pathogens. In rounded (approximate) figures, the world-wide annual production tonnage % lost to various pests at the start of the 21st century have been estimated as follows: losses due to animal pests, 18%; microbial diseases, 16% (and 70-80% of these losses were caused by fungi); weeds, 34%; making a grand total of 68% average annual loss of crop production tonnage (data from Oerke, 2006. Crop losses to pests. The Journal of Agricultural Science, 144(1), 31-43.). Plant pathogens are often divided into biotrophs, necrotrophs, and hemibiotrophs according to their lifestyles. The definitions of these terms are as follows: biotrophs derive energy from living cells. They are found on (e.g., also insects feeding on a plant) or in living plants and can have very complex nutrient requirements. Further, they do not kill host plants rapidly. Necrotrophs derive energy from killed cells. They invade and kill plant tissue rapidly and then live saprotrophically on the dead remains. Finally, hemibiotrophs have an initial period of biotrophy followed by necrotrophy.

In another embodiment, the pathogen against which an increased resistance of a plant is desired, may thus be a biotrophic, necrotrophic or hemibiotrophic fungus, preferably selected from the group consisting of: Zymoseptoria tritici, Setosphaeria turcica, Fusarium spp. Fusarium graminearum, Colletotrichum spp. such as Colletotrichum graminicola, Magnaporthe grisea, Magnaporthe oryzae, Phytophthora infestans, Cercospora spp., preferably Cercospora beticola or Cercospora zeae-mayidis.

Plant pathogens can have a broad host range, for example in the case of insects feeding in different plant species with the same preference, or they may have a rather narrow host range, e.g., in the case of plant viruses. A plant pathogen according to the present invention may thus include any plant pathogen against which resistance can be increased by modulation of a CPL3 gene sequence, a regulatory sequence thereof, a transcript thereof or a protein product thereof.

In certain embodiments, the pathogen against which an increased resistance may be obtained may be a wheat or maize pathogen as represented in the following Tables 1 to 7:

TABLE 1
Fungal diseases and corresponding pathogens of Triticum spp.

| | | Pathogen | Disease |
|---|---|---|---|
| Pathogen | Disease | type | type |
| Blumeria graminis f. sp. tritici | Powdery mildew | Fungus | Foliar disease |
| Drechslera tritici-repentis | Tan spot | Fungus | Foliar disease |
| Fusarium culmorum | Fusarium head blight | Fungus | Head disease |
| Fusarium graminearum | Fusarium head blight | Fungus | Head disease |
| Gaeumannomyces graminis var. tritici | Various diseases | Fungus | Root disease |
| Magnaporthe olyzae | Wheat blast | Fungus | Head disease |
| Pseudocercosporella herpotrichoides | Eyespot | Fungus | Stem disease |
| Puccinia graminis f. sp. tritici | Black rust | Fungus | Foliar and stem disease |
| Puccinia striiformis f. sp. tritici | Yellow rust | Fungus | Foliar disease |
| Puccinia triticina f. sp. tritici | Brown rust | Fungus | Foliar disease |
| Zymoseptoria tritici | Septoria leaf blotch | Fungus | Foliar disease |
| Stagonospora nodorum | Stagonospora nodorum blotch | Fungus | Foliar and head disease |

TABLE 2
Fungal diseases and corresponding pathogens of Zea mays

| | | Pathogen | Disease |
|---|---|---|---|
| Pathogen | Disease | type | type |
| Aspergillus flavus | Aspergillus ear rot | Fungus | Ear disease |
| Aspergillus parasiticus | Aspergillus ear rot | Fungus | Ear disease |
| Aureobasidium zeae | Eyespot | Fungus | Foliar disease |
| Bipolaris maydis | Southern corn leaf blight | Fungus | Foliar, stalk and ear disease |
| Bipolaris zeicola | Northern corn leaf spot | Fungus | Foliar and ear disease |
| Cercospora zeae-maydis | Gray leaf spot | Fungus | Foliar disease |
| Colletotrichum graminicola | Anthracnose leaf blight | Fungus | Foliar, stalk and ear disease |
| | Anthracnose stalk rot | | |
| | Anthracnose ear rot | | |
| Fusarium graminearum | Gibberella crown and stalk rot | Fungus | Crown, stalk and ear disease |
| | Gibberella ear rot | | |
| Fusarium proliferatum | Fusarium stalk and ear rot | Fungus | Stalk and ear disease |
| Fusarium subglutinans | Fusarium stalk and ear rot | Fungus | Stalk and ear disease |
| Fusarium temperatum | Fusarium stalk and ear rot | Fungus | Stalk and ear disease |
| Fusarium verticillioides | Fusarium ear rot | Fungus | Ear disease |
| Macrophomina phaseolina | Charcoal rot | Fungus | Stalk disease |
| Penicillium species | Penicillium ear rot | Fungus | Ear disease |
| Phaeospaeria maydis | Phaeospaeria leaf spot | Fungus | Foliar disease |
| Phoma terrestis, Phytium species | Red root rot | Fungus | Root and stalk disease |
| and Fusarium species | | | |
| Physoderma maydis | Physoderma brown spot and stalk rot | Fungus | Foliar and stalk disease |
| Puccinia polysora | Southern rust | Fungus | Foliar disease |
| Puccinia sorghi | Common rust | Fungus | Foliar disease |
| Rhizoctonia solani | Rhizoctonia crown and brace root rot | Fungus | Seedling and root disease |
| Rhizoctonia solani f. sp. | Banded leaf and | Fungus | Foliar disease |
| sasakii | sheath blight | | |
| Setosphaeria turcia | Northern corn leaf blight | Fungus | Foliar disease |
| Sphacelotheca reiliana | Head smut | Fungus | Ear disease |
| Stenocarpella macrospora | Diplodia leaf streak | Fungus | Foliar disease |
| Stenocarpella maydis | Diplodia stalk rot | Fungus | Stalk and ear |
| | Diplodia ear rot | | disease |
| Trichoderma viride | Trichoderma ear rot | Fungus | Ear disease |
| Ustilago maydis | Common smut | Fungus | Foliar disease |

TABLE 3
Oomycete diseases of Zea mays

| | | Pathogen | |
|---|---|---|---|
| Pathogen | Disease | type | Disease type |
| Peronosclerospora sorghi | Sorghum downy mildew | Oomycete | Foliar disease |
| Phythium aphanidermatum | Phytium stalk rot | Oomycete | Stalk disease |
| Phythium species | Pythium seedling blight and root rot | Oomycete | Seedling and root |
| Sclerophthora macrospora | Crazy top | Oomycete | Foliar disease |

TABLE 4
Bacterial diseases of Zea mays

| | | Pathogen | Disease |
|---|---|---|---|
| Pathogen | Disease | type | type |
| Clavibacter michiganensis | Goss’s wilt | Bacterium | Foliar disease |
| Erwinia species | Bacterial stalk rot | Bacterium | Stalk disease |
| Pantoea stewartii | Stewart's disease | Bacterium | Foliar and stalk |
| | | | disease |
| Pseudomonas syringae | Holcus leaf spot | Bacterium | Foliar disease |
| pv. syringae | | | |

TABLE 5
Viral diseases of Zea mays

| Pathogen | Disease | Pathogen type | Disease type |
|---|---|---|---|
| Maize dwarf mosaic virus | Maize dwarf mosaiv | Virus | Foliar disease |
| Maize chlorotic dwarf virus | Maize chlorotic dwarf | Virus | Foliar disease |
| Maize rough dwarf virus | Maize rough dwarf | Virus | Foliar disease |
| Maize streak virus | Maize Streak | Virus | Foliar disease |

TABLE 6
Nematode diseases of Zea mays

| | | Pathogen | Disease |
|---|---|---|---|
| Pathogen | Disease | type | type |
| Belonolaimus and | Sting and needle | Nematode | Root disease |
| Longidorus species | neamtodes | | |
| Meloidogyne species | Root-knot nematode | Nematode | Root disease |
| Paratrichodorus specis | Stubby-root nematode | Nematode | Root disease |
| Pratylenchus species | Root-lesion nematode | Nematode | Root disease |

TABLE 7
Insect diseases of Zea mays

| | | Pathogen | Disease |
|---|---|---|---|
| Pathogen | Disease | type | type |
| Agrotis ipsilon | Cutworm | Insect | Leaf disease |
| Ostrinia nubilalis | European | Insect | Stalk and ear |
| | corn borer | | disease |
| Pseudaletia unipuncta | Armyworm | Insect | Leaf and ear |
| | | | disease |
| Rhopalosiphum maidis | Corn leaf aphid | Insect | Leaf disease |

The terms “resistance” or “resistant” as used herein refers to the capacity of a plant to resist to the phenotype as caused by infestation with a pathogen, preferably a fungal pathogen to a certain degree, i.e., the prevention, reduction or delay of an infection or harm caused by the pathogen. “Resistance”, therefore, does not exclusively refer to a “black or white” phenotype, but is intended to mean any improvement of infection or infestation symptoms as observed for a plant having an endogenous CPL3 protein activity in comparison to a plant having a specifically modified CPL3 activity according to the various aspects of the present disclosure. Resistance to a given pathogen can thus range from a slightly increased resistance to an absolute resistance towards a given pathogen always comparing the modified plant, plant cell, tissue, organ or material to a naturally occurring non modified plant, plant cell, tissue, organ or material, respectively.

In one embodiment, the pathogen resistance may be a fungal resistance, more preferably a hemibiotrophic fungal resistance. Generally, there is a great need to identify new anti-fungal strategies. Hemibiotrophic pathogens cover some of the most relevant pathogens for crop plants, e.g., wheat (Triticum aestivum), soybean (Glycine max), corn (Zea mays) etc. The hemibiotrophic fungal pathogen Exserohilum turcicum (anamorph form of the fungus; teleomorph: Setosphaeria turcica) causing NCLB, for example, is found in humid climates wherever corn is grown and has bipartite life cycle hampering the establishment of efficient anti-fungal agents protecting plants. E. turcicum survives in debris of Zea mays and builds up over time in high-residue and continuous corn cropping systems. High humidity and moderate temperatures favor the persistence of the E. turcicum fungus causing tremendous yield losses, e.g., due to decreased photosynthesis resulting in limited ear fill, or harvest losses if secondary stalk rot infection and stalk lodging accompany loss of leaf area. Due to their complicated life cycle, hembibiotrophic pathogens are hard to combat and represent a huge threat in agriculture as these fungi can often evade the plant immune system. Therefore, strategies, preferably other than relying on fungicides, are needed for providing relevant crop plants having an endogenous resistance to selected pathogens like fungal pathogens.

In one embodiment, the one or more mutation(s) to be introduced into a CPL3 encoding gene, or a regulatory sequence thereof, may have a dominant negative effect and may be present in the heterozygous state in the plant. In another embodiment, the mutation may be present in the homozygous state. Depending on the amount of different CPL3 alleles in a germplasm, and further depending on the function outcome, a homozygous or a heterozygous state may be preferably to obtain an optimum balance between increased fungal resistance and a maintenance of normal cellular functions.

After discovery of the CPL3 homologues and the tests on favorable mutations, the possibility was tested whether downregulation of CPL3 gene expression leads to improved pathogen resistance. Therefore, maize (Zea mays) ZmCPL3 (SEQ ID NO: 9 and 19) and wheat (Triticum aestivum) TaCPL3-A (SEQ ID NO: 5 and 15), TaCPL3-B (SEQ ID NO: 6 and 16) and TaCPL3-D (SEQ ID NO: 7 and 17) genes were selected for pathogen resistance tests to obtain functional data for relevant crop plants based on the genes identified.

It was surprisingly observed that the specific modulation of a CPL3 protein or a nucleic acid sequence encoding the same or encoding the regulatory sequence for a cp13 gene resulted in a plant cell, tissue, organ, whole plant, or plant material showing overall normal growth and/or proliferation, either for the settings using a dominant negative mutation, a modification of a regulatory sequence, or an incomplete down-regulation of a CPL3 transcript. “Normal growth and proliferation” is meant to imply that a plant cell or organism modulated according to the present disclosure substantially shows the same growth and proliferation characteristics as a not modulated plant or plant cell on a phenotypic level. For example, the modulated plant does not show detectable symptoms associated with growth, cell division, or cell death in direct comparison to a non-modulated material of same origin and with the same genetic background. In view of the fact that CPL3 proteins are important enzymes in cell signaling, this finding was not expected and is likely associated with the way the CPL3 signaling is modulated in a rather specific way according to the present invention relying on specific mutations and/or specific downregulation of expression of CPL3 instead of providing a full knock out of the respective genes in a heterozygous or homozygous state. In particular, it was observed as significant advantage of the present invention that an incomplete down-regulation or a targeted mutation of a CPL3 gene sequence or a regulatory sequence thereof besides the desired effect of achieving pathogen resistance does not disturb normal plant growth and/or development.

The term “modulation” or “modulating” is used herein as a superordinate term for a targeted control or modification of a naturally occurring DNA, RNA, or protein sequence, including the control or modification of transcription, translation or post-translational events. According to the various aspects of the present disclosure, a “mutation” can be understood as specific form of a modulation acting on DNA as target nucleic acid sequence to establish a potentially inheritable modulation. A mutation can be introduced in a targeted way, e.g., by relying on a site-directed DNA modifying enzyme, or a mutation can be introduced in a random manner followed by specific screening, e.g., by TILLING, the latter method allowing a higher throughput, but demanding more screening for identifying desired mutations.

In one embodiment, there may be provided a plant having increased pathogen resistance, wherein the one or more mutation(s) of the nucleotide sequence encoding a CPL3 protein may cause the substitution of Asp by Ala at position 928 referenced to SEQ ID NO: 19, at position 944 referenced to SEQ ID NO: 14, at position 949 referenced to SEQ ID NO: 11, at position 944 referenced to SEQ ID NO: 14, at position 949 referenced to SEQ ID NO: 11, at position 953 referenced to SEQ ID NO: 12, at position 910 referenced to SEQ ID NO: 13, at position 890 referenced to SEQ ID NO: 18, at position 938 referenced to SEQ ID NO: 15, at position 929 referenced to SEQ ID NO: 16, at position 938 referenced to SEQ ID NO: 17.

In another embodiment, the plant having pathogen resistance can be obtain by using one or more silencing construct(s) comprising (l.) an RNAi molecule directed against, targeting, or hybridizing with the nucleotide sequence encoding the CPL3 protein, or a polynucleotide sequence encoding said RNAi molecule; or (II.) an RNA-specific CRISPR/Cas system directed against or targeting the nucleotide sequence encoding the CPL3 protein, or a polynucleotide sequence encoding said RNA-specific CRISPR/Cas system, preferably wherein the RNAi molecule is selected from a dsRNA molecule, a shRNA molecule, a miRNA molecule or a siRNA molecule which comprises at least 15, 16, 17, 18, 19, 20, 21, 22, 23, 24, 25, 26, 27, 28, 29, 30, 31, 32, 33, 34, 35, 36, 37, 38, 39, 40, 45 or 50 contiguous nucleotides of the coding nucleotide sequence of the CPL3 protein or the complementary sequence thereof in sense or antisense direction, more preferably wherein the RNAi molecule is selected from a sequence of SEQ ID NOs: 21 to 24, or a sequence having at least 95%, 96%, 97%, 98% or 99% sequence identity thereto.

In a preferred embodiment, the RNAi molecule or a sequence comprised by a silencing construct does preferably not share substantial sequence identity with other genomic regions in the genome of the plant. “Substantial sequence identity” implies that a RNAi molecule, or a silencing construct encoding or comprising the same, would have identity to a sequence other than the target sequence in the genome or transcriptome of a plant or plant cell. Based on the disclosure herein and further based on the genomic data of relevant crop plants, the skilled person can thus create silencing constructs or RNAi molecules for knock-down experiments of CPL3 transcripts which will be highly specific for a CPL3 target sequence to allow an otherwise normal cellular function.

There are multiple ways to downregulate the expression of a gene. Among them are the expression of RNAi or microRNA constructs or the modification of the promoter or other regulatory elements of a gene. In one embodiment for downregulation of CPL3 genes, in accordance with the second aspect of the modes of modulation according to the present disclosure, a dominant negative allele of CPL3 may be expressed. Fukudome et al. (2014) reported that the point mutation D128A in the conserved DXDT motif of the catalytic phosphatase domain resulted in in a dominant-negative form, at least for the Arabidopsis protein AtCPL4 that is involved in normal growth and plant development. Overexpression of the dominant allele AtCPL4_D128A in Arabidopsis, however, was lethal and resembled the phenotype of AtCPL4 knock out lines that are homozygous lethal. Arabidopsis plants with AtCPL4 RNAi constructs were viable with mild toxicity phenotype. This shows that strong overexpression of a dominant negative AtCPL4 allele resembles a complete knock out of AtCPL4.

To achieve a fine-tuned modulation according to the first aspect of the modes of modulation according to the present disclosure, a dominant negative allele may be provided by introducing at least one targeted mutation into at least one CPL3 protein encoding sequence, wherein the at least one mutation results in a dominant negative CPL3 allele, preferably wherein the mutation causes an alteration of the amino acid sequence of a conserved DXDXT domain of a CPL3 protein. According to one embodiment, the mutant variant of the respective CPL3 variant may then be put under the control of a weak promoter, e.g., an endogenous promoter, optionally an inducible promoter according to the various methods of generating a plant cell, tissue, organ, whole plant, or plant material to achieve pathogen resistance, preferably fungal resistance, by simultaneously avoiding potentially lethal side effects of a strong expression of the variant.

In one embodiment, the promoter may be an endogenous or native promoter.

Therefore, one embodiment covers a dominant negative allele of the CPL3 gene under the control of a native promoter which confers pathogen resistance, preferably resistance against hemibiotrophic pathogens. To gain dominant negative alleles of the discovered CPL3 genes, mutations for example in the DXDXT motif similar to D128A in AtCPL4 may be used. Further preferred point mutations that lead to a dominant negative allele are selected from the group consisting of D928A in ZmCPL3 (SEQ ID NO: 19), D944A in BvCPL3 (SEQ ID NO: 14), D949A in GmCPL3_1 (SEQ ID NO: 11), D953A in GmCPL3_2 (SEQ ID NO: 12), D910A in StCPL3 (SEQ ID NO: 13), D890A in SbCPL3 (SEQ ID NO: 18), D938A in TaCPL3-A (SEQ ID NO: 15), D929A in TaCPL3-B (SEQ ID NO: 16), and D938A in TaCPL3-D (SEQ ID NO: 17). Based on the present disclosure, comparable mutations can be inserted at comparable positions in the conserved DXDXT motif of further CPL gene variants in further plants, preferably crop plants.

In another embodiment, a non-native promoter may be inserted to further control the expression of the CPL3 gene of interest in a target plant of interest.

In a further embodiment, downregulation of at least one CPL3 gene can be achieved by introducing a point mutation into at least one native CPL3 gene by means and techniques further disclosed below that leads to a dominant-negative allele and to keep this mutation in a heterozygous state. The resistance effect of a dominant-negative allele of CPL3 would be genetically dominant which has benefits in breeding of resistant hybrid crops as compared to recessive mutations in the promoter, for example, that would need to be present in both parents of the hybrid.

For plants or plant cells, where paralogs of CPL3 genes are present, like in soybean (Glycine max), embodiments using a dominant-negative CPL3 allele for engineering pathogen resistant plants may be preferred as this strategy potentially requires less effort than, for example, downregulating all CPL3 paralogs by promoter modifications or by silencing constructs as disclosed herein at the same time.

In the context of the present disclosure, the terms “RNA interference” or “RNAi” refer to a gene down-regulation mechanism meanwhile demonstrated to exist in all eukaryotes. The mechanism was first recognized in plants where it was called “post-transcriptional gene silencing” or “PTGS”. In RNAi, small RNAs (of about 21-24 nucleotides) function to guide specific effector proteins (e.g., members of the Argonaute protein family) to a target nucleotide sequence by complementary base pairing. The effector protein complex then down-regulates the expression of the targeted RNA or DNA. Small RNA-directed gene regulation systems were independently discovered (and named) in plants, fungi, worms, flies, and mammalian cells. Collectively, PTGS, RNA silencing, and co-suppression (in plants); quelling (in fungi and algae); and RNAi (in Caenorhabditis elegans, Drosophila, and mammalian cells) are all examples of small RNA-based gene regulation systems.

In plants, during RNAi mechanism, silencing initiates with the enzyme Dicer and dsRNA is processed to convert the silencing trigger to ˜22-nucleotide, small interfering RNAs (siRNAs). The antisense strand of siRNA become specific to endonuclease-protein complex, RNA-induced silencing complex (RISC), which then targets the homologous RNA and degrade it at specific site that results in the knock-down of protein expression. RNAi technology may thus be a substitute of complex molecular techniques because of containing several benefits: its specificity and sequence-based gene silencing. Plants can also control viral diseases by RNAi and reveal resistance when having proper anti-sense or hairpin RNAi constructs. In plants, specifically to achieve pathogen resistance, hairpin (hp) dsRNA including small hairpin RNA (shRNA), self-complementary hpRNA, and intron-spliced hpRNA can be formed in vivo using inverse repeat sequences from viral genomes. Among these, PTGS with the highest efficiency was elicited by the method involving self-complementary hairpin RNAs separated by an intron. High resistance against viruses has been observed in plants even in the presence of inverted repeats of dsRNA-induced PTGS (IR-PTGS).

Meanwhile, a variety of different RNAi constructs to be used as silencing construct to be used according to the various aspects and embodiments of the present disclosure are available to the skilled person (Younis et al., Int J Biol Sci. 2014; 10(10): 1150-1158). Several methods to induce RNAi, RNAi vectors, in vitro dicing and synthetic molecules are reported. Mechanistically, introduction of short pieces of double stranded RNA (dsRNA) and small or short interfering RNA (SIRNA) into the cytosol, may initiate the pathway culminating targeted degradation of the specific cellular mRNA, i.e., the target mRNA of the gene transcript to be silenced according to the present invention. Another RNAi molecule are micro RNAs or miRNAs. In spite of similarity in size (20-24 nt), miRNA differ from siRNA in precursor structures, pathway of biogenesis, and modes of action. Artificial miRNAs are known to the skilled person. Both, miRNAs and siRNAs are known to be important regulators of gene expression in plants.

In another embodiment, an RNAi and self-cleaving hammerhead ribozyme may be used to achieve a desired modulation, also on a DNA level (Li Z., Rana T.M. Therapeutic targeting of microRNAs: current status and future challenges. Nat Rev Drug Discov. 2014; 13(8): 622-638.). These reagents allow for targeted control of gene expression by promoting the removal of specific mRNAs from the cytoplasm. The hammerhead ribozyme (HHR), first seen in tobacco ringspot virus satellite RNA, is an example of small nucleolytic RNA molecules capable of self-cleavage (i.e., the name ribozymes). Other autocatalytic (self-cleaving type) small RNA molecules are twister, twister sister, pistol, and hatchet ribozyme. HHRs are composed of a conserved central sequence with three radiating helical domains. Natural HHRs are not true ribozymes as they are only capable of carrying out a single self-cleavage reaction. Synthetic HHRs have been engineered to overcome this by separating the HHR into two components: ribozyme (the part of the HHR which remains unchanged) and substrate (the target sequence that will be cleaved). Another class of suitable modulators for the purpose of the present disclosure are riboswitches. Riboswitches are RNA elements that modulate mRNA expression through binding of a ligand, which is typically a small organic molecule or ion, to its aptamer domain. In one embodiment, the use of a riboswitch might be of interest to modify CPL3 expression in a tightly controlled manner. Meanwhile, a variety of ribozymes and riboswitches types including DNAzymes and temperature-sensitive ribozymes is available to the skilled person (Guha TK, Wai A, Hausner G. Programmable Genome Editing Tools and their Regulation for Efficient Genome Engineering. Comput Struct Biotechnol J. 2017; 15:146-160. Published 2017 Jan. 12. doi: 10.1016/j.csbj.2016.12.006).

The silencing construct may thus be an RNAi silencing construct. The silencing construct may be presented as vector for expression in a cell of interest, or the silencing construct can be prepared ex vivo to be added to a cell, material, tissue, organ or whole organism of interest. In one embodiment, the silencing construct may be operatively linked to a constitutively active promoter. In another embodiment, the silencing construct may be operatively linked to an inducible promoter to control expression of the construct depending on an inducer. Controlled expression of the silencing construct can allow targeted regulation of expression levels of a target protein of interest to be silenced in a temporal (e.g., only during a certain phase of plant development) and/or spatial (e.g., certain plant organs, tissues, cells, or special compartments/organelles) manner. In particular, due to the fact that the target sequences to be silenced play a critical role in plant immunity, it may have significant advantages to restrict silencing in a tempo-spatial and dose dependent way to avoid severe negative effects of the knock-out of plant immunity effectors like CPL proteins due to their highly relevant roles in defence and development.

In a preferred embodiment, a silencing construct of the present invention may be introduced in a transient manner which additionally guarantees that no genetic material is introduced into a plant or plant cell in an inheritable way.

In yet another embodiment, the silencing construct or the RNAi molecule does not share substantial sequence identity with other genomic regions in the genome of the plant cell, tissue, organ, whole plant, or plant material according to the present disclosure is to be understood as a molecule designed in silico based on the information of a sequence to be silenced in combination with the information of the genome to be modified so that the RNAi molecule does not comprise long stretches of identity to other regions in the genome other than the region to be modulated to avoid off-target effects. Usually, the identity to the sequence to be silenced will thus be very high, i.e., at least 90%, 91%, 92%, 93%, 94%, and more preferably at least 95%, 96%, 97%, 98% or even higher than 99%. The substantial identity to other genomic regions in the genome of the plant cell, tissue, organ, whole plant, or plant material will usually be below 25 bp, preferably below 20 bp, 19 bp, 18 bp, 17 bp, 16 bp, more preferably below 15 bp, 14 bp, 13 bp, 12 bp, 11 bp and most preferably below 10 bp of contiguous stretches aligning with another region of a genome of interest.

In another embodiment, a plant having pathogen resistance may be obtained by modification of the native regulatory sequence(s), wherein the modification may be a transient or stable modification of a regulatory sequence, preferably wherein (i) the modification is introduced by a site-directed DNA modifying enzyme, or wherein (ii) a modified site-directed DNA modifying enzyme mediates the modification, preferably the inhibition, of a regulatory sequence, or wherein (iii) the modification is introduced by random mutagenesis, preferably wherein the random mutagenesis is selected from chemical-induced mutatgenesis or irradiation-induced mutagenesis. Depending on the plant to be modified, both site-directed and random mutagenesis, or a combination thereof, can represent suitable options.

According to the present disclosure, at least one site-directed DNA or RNA modifying enzyme (SDE), or a sequence encoding the same, or a complex comprising the same, can be utilized to modify a CPL3 gene, or a regulatory sequence of a CPL3 gene, or a CPL3 encoded RNA sequence, in a targeted way by at least one SDE, or a catalytically active fragment thereof, or a complex comprising a SDE, or a nucleic acid sequence encoding the same. Targeted genome editing has meanwhile become a powerful genetic tool for studying gene function or for modifying genomes in a precise way. Genome editing tools include meganucleases, zinc finger nucleases (ZFNs), transcription activator-like effector nucleases (TALENs), clustered regularly interspaced short palindromic repeat (CRISPR)-associated nuclease Cas9, and targetrons (Guha et al., supra). SDEs and related constructs or tools relevant to achieve a targeted genome editing event in a given genome are known to the skilled person and can be adapted to a target cell of interest. All of the aforementioned tools can achieve precise genetic modifications by inducing targeted DNA double-strand breaks (DSBs). Depending on the cell cycle stage, as well as the presence or absence of a repair template with homologous terminal regions, the DSB may then be repaired by either non-homologous end joining repair system (NHEJ), or the homologous recombination-based double-strand break repair pathway (HDR).

According to the present disclosure, the at least one site-directed DNA modifying enzyme may thus be selected from at least one of a meganuclease, a ZFN, a TALEN, an Argonaute protein, wherein non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof, RNA-guided nucleases, wherein non-limiting examples of RNA-guided nucleases include the CRISPR associated nucleases, such as Casl, CaslB, Cas2, Cas3, Cas4, Cas5, Cas6, Cas7, Cas8, Cas9 (also known as Csnl and Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csx16, CsaX, Csx3, Csxl, Csxl5, Csfl, Csf2, Csf3, Csf4, Cpfl, CasX, CasY, Mad7, homologs thereof, or modified versions thereof and engineered RNA-guided nucleases (RGNs), a restriction endonuclease, including Fokl or a variant thereof, a recombinase, or two site-specific nicking endonucleases, or a base editor, or any variant or catalytically active fragment of the aforementioned effectors, wherein the at least one site-directed DNA modifying enzyme induces a genome modification such as a double-stranded DNA break (DSB) or single-strand DNA break, or a targeted nucleotide exchange at the target site of a genomic sequence. In some embodiments, breaks or nicks in the target DNA sequence are repaired by the natural processes of homologous recombination (HR) or non-homologous end-joining (NHEJ). In some embodiments, sequence modifications occur at or near the cleaved or nicked sites, which can include deletions or insertions that result in modification of the nucleic acid sequence, or integration of exogenous nucleic acids by homologous recombination or NHEJ.

In one embodiment according to the aspects of the present disclosure directed to the targeted mutation of at least one nucleotide sequence encoding a CPL3 protein, or directed to the modification of a regulatory sequence of at least one CPL3 protein encoding sequence, the at least one site-directed DNA modifying enzyme is a CRISPR-based nuclease, wherein the CRISPR-based nuclease comprises a site-specific DNA binding domain, wherein the at least one CRISPR-based nuclease, or the nucleic acid sequence encoding the same, is selected from the group comprising (a) Cas9, including SpCas9, SaCas9, SaKKH-Cas9, VQR-Cas9, St1Cas9, (b) Cpf1, including AsCpf1, LbCpf1, FnCpf1, (c) CasX, or (d) CasY, or any variant or derivative of the aforementioned CRISPR-based nucleases, optionally wherein the at least one CRISPR-based nuclease comprises a mutation in comparison to the respective wild-type sequence so that the resulting CRISPR-based nuclease is converted to a single-strand specific DNA nickase, or to a DNA binding effector lacking all DNA cleavage ability.

In other embodiments, a CRISPR-Cas13 RNA editing complex may be used to alter the RNA coding potential in a programmable manner which allows a targeted knockdown of endogenous transcripts, preferably CPL3 transcripts, with comparable levels of knockdown as RNAi. Further, Cas13 or dead Cas13 comprising constructs can be used to exchange a RNA base, not only to achieve a transient knock-down. Additionally, RNA editing platforms are available comprising both an RNA knockdown and an RNA editing tool (Cox et al., Science. 2017 Nov. 24; 358 (6366): 1019-1027). As reported for RNAi constructs above, the modulation on RNA level may allow a temporally controlled modulation of expression levels and may have advantages over the creation of knock-outs, in particular due to the fact that CPL3 and CPL3 homologs represent central molecules in plant immunity so that a full knock-out or an uncontrolled modulation may result in undesired side effects or even cell death.

Another class of genome editing tools suitable for the various embodiments of the present invention are base editors.

Base editors, including BEs (base editors mediating C to T conversion) and ABEs (adenine base editors mediating A to G conversion), are powerful tools to introduce direct and programmable mutations of all four transitions to the DNA without the need for double-stranded cleavage (Komor et al., Nature, 2016, 533(7603), 420-424; Gaudelli et al., Nature, 2017, 551, 464-471). In general, base editors are composed of at least a DNA targeting module and a catalytic domain that deaminates cytidine or adenine. There are three BE versions described in Komor et al., 2016 (vide supra), namely BE1, BE2 and BE3, with BE3 showing the highest efficiency of targeted C to T conversion, resulting in up to 37% of desired C to T conversion in human cells. BE3 is composed of APOBEC-XTEN-dCas9(A840H)-UGI, where APOBEC1 is a cytidine deaminase, XTEN is 16-residue linker, dCas9(A840H) is a nickase version of Cas9 that nicks the non-edited strand and UGI is an Uracil DNA glycosylase inhibitor. In this system, the BE complex is guided to the target DNA by the sgRNA, where the cytosine is then converted to uracil by cytosine deamination. The UGI inhibits the function of cellular uracil DNA glycosylase, which catalyzes removal of uracil from DNA and initiates base-excision repair (BER). Nicking of the unedited DNA strand helps to resolved the U:G mismatch into desired U:A and T:A products.

ABEs were first developed by Gaudelli et al., 2017 (supra) for converting A-T to G-C. A transfer RNA adenosine deaminase was evolved to operate on DNA, which catalyzes the deamination of adenosine to yield inosine, which is read and replicated as G by polymerases. By fusion of the evolved adenine deaminase and a Cas9 module, ABEs described in Gaudelli et al., 2017 (supra) showed about 50% efficiency in targeted A to G conversion.

All four transitions of DNA (A-T to G-C and C-G to T-A) are possible as long as the base editors can be guided to the target place. Base editors convert C or A at the non-targeted strand of the sgRNA.

According to the present disclosure, the BE may be specifically optimized for use in a plant cell system, including the use of codon-optimized sequences for a plant or plant cell of interest, and further including the use of a plant specific promoters, for example, an ubiquitin promoter, in case the construct is provided as expression cassette.

In one embodiment according to the various aspects of the present invention, the aspect of modulating a nucleotide sequence encoding an endogenous C-terminal domain phosphatase-like 3 (CPL3) protein or a regulatory sequence thereof can be achieved by specifically combining transient and stable modulation techniques, i.e., by combining any one of introducing as alternative (i) one or more mutation(s) of the nucleotide sequence encoding a CPL3 protein, preferably wherein the one or more mutation(s) has/have a dominant negative effect, preferably wherein the one or more mutation(s) cause(s) an alteration of the amino acid sequence of the conserved catalytic domain of the CPL3 protein comprising the DXDXT/V motif; and/or introducing as alternative (ii) one or more silencing construct(s) directed to one or more endogenous nucleotide sequence(s) encoding a CPL3 protein, preferably directed to all endogenous nucleotide sequences encoding a CPL3 protein; and/or introducing as alternative (iii) a modification of the native regulatory sequence(s) of one or more nucleotide sequence(s) encoding an endogenous CPL3 protein, preferably of all native regulatory sequence(s) of the nucleotide sequences encoding an endogenous CPL3 protein, wherein the modification causes a reduced expression rate of the one or more nucleotide sequence(s) encoding an endogenous CPL3 protein.

For example, in one embodiment, one CPL3 allele, or a regulatory sequence thereof, or a RNA transcript thereof, in a polyploid plant may be stably mutated, wherein another CPL3 allele, or a regulatory sequence thereof, may be transiently modified by a silencing construct according to the present invention. Depending on the total amount of different CPL3 alleles present in a germplasm, this strategy can provide the best dosage effect to achieve optimum pathogen resistance, whilst maintaining normal plant growth and development characteristics as mediated by CPL3 alleles or the proteins encoded thereof and the corresponding regulatory sequences.

In another embodiment, different CPL3 alleles, or the regulatory sequences thereof, or a RNA transcript thereof, may be targeted by the same of the above described alternatives (i) to (iii), depending on the plant and their CPL3 genotype to be modified.

Therefore, any of the above alternatives (i) to (iii) may be used alone or in combination, either simultaneously, or subsequently, wherein subsequently may include the subsequent introduction into the same plant or plant cell, but it may also include the subsequent use in different plant or plant cell generations. For example, the first modulation can be achieved in a first plant or plant cell. Next, a progeny of said plant or plant cell may be obtained and the subsequent introduction according to any of the above alternatives (i) to (iii) may then be an introduction into the progeny plant or plant cell.

In certain embodiments, fusion molecules comprising one or more of the modulation tools according to alternatives (i) to (iii) may be used.

For certain applications, transient and/or non-transgenic methods and modes of introduction of the various constructs according to the present disclosure may be preferred.

In one embodiment according to the various aspects of the present invention, the modification or mutation may be performed by oligonucleotide directed mutagenesis (ODM), chemical mutagenesis, e.g., TILLING, for example, by applying an efficient amount of a mutagenic agent, preferably ethylmethane sulfonate, N-ethyl-N-nitrosourea, or by radiation.

TILLING, initially a functional genomics tool in model plants, has been extended to many plant species and become of paramount importance to reverse genetics in crops species. A major recent change to TILLING has been the application of next-generation sequencing (NGS) to the process, which permits multiplexing of gene targets and genomes. NGS will ultimately lead to TILLING becoming an in silico procedure. Because it is readily applicable to most plants, it remains a dominant non-transgenic method for obtaining mutations in known genes and thus represents a readily available method for non-transgenic approaches according to the methods of the present invention. As it is known to the skilled person, TILLING usually comprises the chemical mutagenesis, e.g., using ethyl methanesulfonate (EMS), N-ethyl-N-nitrosourea, or UV light induced modification of a genome of interest, together with a sensitive DNA screening-technique that identifies single base mutations in a target gene, or a regulatory sequence thereof. The skilled person can thus define an efficient amount of a mutagenic agent to obtain a sufficient number of mutagenic events whilst maintaining genomic integrity for a given plant genome of interest.

SSNs and ODM mutagenesis both are suitable techniques for precision genome engineering in plant cells as well and are suitable to induce a modification or mutation according to the various aspects of the present disclosure. As it is known to the skilled person, ODM offers a rapid, precise and non-transgenic breeding alternative for trait improvement in agriculture to address this urgent need. ODM is a precision genome editing technology, which uses oligonucleotides to make targeted edits in plasmid, episomal and chromosomal DNA of bacterial, fungal, mammalian and plant systems.

According to another aspect, a cell, tissue, organ, seed or material of a plant according to the various aspects and embodiments disclosed herein may be obtained or may be used as starting point for obtaining a pathogen resistant plant, cell, tissue, organ, seed or material of a plant.

In a second aspect, a nucleic acid molecule comprising a nucleotide sequence encoding for a C-terminal domain phosphatase-like 3 (CPL3) protein, wherein the nucleotide sequence is selected from the group consisting of: (a) a nucleotide sequence set forth in SEQ ID NOs: 2-10or a homologous, orthologous or paralogous sequence thereof; (b) nucleotide sequence having at least 80%, at least 85%, at least 90%, at least 92%, at least 94%, at least 95%, at least 96%, at least 97%, at least 98% or at least 99% identity to one of the nucleotide sequences as defined in (a), or (c) a nucleotide sequence encoding for an amino acid sequence set forth in SEQ ID NOs: 11-19; (d) a nucleotide sequence encoding for an amino acid sequence having at least 80%, at least 85%, at least 90%, at least 92%, at least 94%, at least 95%, at least 96%, at least 97%, at least 98% or at least 99% identity to one of the sequences set forth in SEQ ID NOs: 11-19, or (e) a nucleotide sequence hybridizing with a nucleotide sequence complementary to the nucleotide sequence as defined in (a)-(d) under stringent conditions, wherein the nucleotide sequence comprises at least one mutation capable of conferring or increasing resistance to a pathogen in plant in which the nucleic acid molecule is expressed, preferably wherein the pathogen may be a hemibiotrophic fungus, more preferably the pathogen is a hemibiotrophic fungus selected from the group consisting of: Zymoseptoria tritici, Setosphaeria turcica, Fusarium spp. Fusarium graminearum, Colletotrichum spp. such as Colletotrichum graminicola, Magnaporthe grisea, Magnaporthe oryzae, Phytophthora infestans, or preferably wherein the pathogen may be a fungus selected from Cercospora spp., preferably Cercospora beticola or Cercospora zeae-mayidis, which may be used to obtain a pathogen resistant plant, cell, tissue, organ, seed or material of a plant.

Cercospora is the cause of leaf spot diseases in various plants, but it also causes disease on: alfalfa, asparagus, banana, brassicas, Cannabis, carrot, celery, cereals, coffee, cucumber, figs, geraniums, grapes, grasses, hazel, hops, lentil, lettuce, mango, millet, orchids, papaya, peanut, pear, peas, peppers, potato, roses, sorghum, soybean, spinach, strawberry, sugar beet, sugarcane (the spots merge into stripes; so the disease is called ‘black stripe’), sycamore, tobacco, watermelon, and many wild plants and ornamentals and thus represents a relevant fungal pathogen.

In one embodiment, the mutation may be a mutation of the nucleotide sequence encoding a CPL3 protein, preferably a mutation having a dominant negative effect, preferably wherein the mutation causes an alteration of the amino acid sequence of the conserved catalytic domain of the CPL3 protein comprising the DXDXT/V motif, more preferably a mutation of the nucleotide sequence encoding a CPL3 protein causing the substitution of Asp by Ala at position 928 referenced to SEQ ID NO: 19, at position 944 referenced to SEQ ID NO: 14, at position 949 referenced to SEQ ID NO: 11, at position 944 referenced to SEQ ID NO: 14, at position 949 referenced to SEQ ID NO: 11, at position 953 referenced to SEQ ID NO: 12, at position 910 referenced to SEQ ID NO: 13, at position 890 referenced to SEQ ID NO: 18, at position 938 referenced to SEQ ID NO: 15, at position 929 referenced to SEQ ID NO: 16, at position 938 referenced to SEQ ID NO: 17.

According to the second and all further aspects of the present disclosure, one or more of the same, or one or more different mutation(s) may be effected depending on the amount and nature of CPL3 alleles present in the genome of a target plant of interest. In certain embodiments, more than one mutation may be desired to obtain a phenotype of optimum pathogen resistance without side effects, like, for example, impeded plant growth.

In a further aspect, a method of generating a plant having pathogen resistance or a plant cell, tissue, organ, seed, or plant material thereof, may be used based on the above findings on CPL3 modulation and its effect on pathogen resistance, wherein the method may comprise the steps of: (i) providing one or more silencing construct(s) according to the embodiments disclosed for the first aspect, or one or more sequences encoding the same; (ii) modifying a plant cell, tissue, organ, plant, seed, or plant material by introducing the one or more silencing construct(s) or the sequence encoding the same of (i), into the genome of said plant cell, tissue, organ, plant, seed, or plant material; and (iii) obtaining the modified plant cell, tissue, organ, plant, seed or plant material, (iv) optionally, regenerating a plant from the plant cell, tissue, organ or plant material or growing a seed on a plant obtained in (iii), wherein the plant cell, tissue, organ, plant, seed or plant material obtained in (iii), the plant regenerated in (iv) or the seed grown in (iv) may comprise the introduced one or more silencing construct(s) or the sequence encoding the same and thereby has pathogen resistance.

In a further aspect, the method of generating a plant having pathogen resistance or a plant cell, tissue, organ, seed, or plant material thereof, may comprise the steps of: (i) providing at least one site-directed DNA modifying enzyme, or a sequence encoding the same, and optionally at least one DNA repair template, wherein the at least one site-directed DNA modifying enzyme and optionally the at least one DNA repair template: (a) may be directed or targeted to the nucleotide sequence encoding the CPL3 protein as defined in the first aspect above; or (b) may be directed or targeted to regulatory sequence of at least one CPL3 protein encoding nucleotide sequence as defined in the first aspect above; (ii) introducing the at least one site-directed DNA modifying enzyme or a sequence encoding the same, and optionally the at least one DNA repair template into the plant cell, tissue, organ, plant, or plant material; (iii) mutating or modifying the nucleotide sequence encoding the CPL3 protein or the regulatory sequence thereof in the genome of the plant cell, tissue, organ, plant, or plant material and obtaining a mutant or modified population of plant cells, tissues, organs, plants, or plant materials; (iv) optionally: screening the population for a dominant negative mutation, thereby conferring or increasing pathogen resistance, or screening the population for a mutation or modification in the nucleotide sequence encoding the CPL3 protein or the regulatory sequence thereof; (v) identifying and thereby obtaining a plant cell, tissue, organ, plant, or plant material having pathogen resistance.

In certain embodiments, a functional fragment or truncated or modified version of a site-directed DNA modifying enzyme, or a sequence encoding the same, may be used.

A mutant or modified population of plant cells implies that at least one cell in a population comprises a targeted mutation or modification, wherein different cells in the population may comprise a different set of mutations or modifications in their respective genomes. The skilled person can easily identify the mutations or modifications as obtained by the various methods disclosed herein using common techniques like PCR etc.

In yet a further aspect, the method of generating a plant having pathogen resistance or a plant cell, tissue, organ, seed, or plant material thereof, may comprise a TILLING approach and may thus comprise the steps of: (i) subjecting the plant cell, tissue, organ, plant, or plant material, preferably seeds of a plant, to an efficient amount of a mutagenic agent, preferably ethylmethane sulfonate, N-ethyl-N-nitrosourea, or radiation, (ii) obtaining a mutagenized population of plant cells, tissues, organs, plants, or plant materials, optionally by growing plants from the mutagenized population; (iii) screening the mutagenized population for pathogen resistance, optionally by isolating and analyzing genomic DNA from the plants having pathogen resistance; (iv) identifying and obtaining a modified plant cell, tissue, organ, plant, or plant material having pathogen resistance.

In still another aspect, a method of generating a plant having pathogen resistance or a plant cell, tissue, organ, seed, or plant material thereof, may comprise the steps of: (i) transforming at least one plant cell with at least one nucleic acid molecule according to the second aspect disclosed herein; and (ii) regenerating and thus obtaining a plant cell, tissue, organ, plant, or plant material having pathogen resistance.

Depending on the pathogen and the plant of interest, the skilled person can identify suitable assays to determine whether the modulation according to the present invention is suitable to increase pathogen resistance.

Any screening according to the various aspects and embodiments disclosed herein may, for example, be done by means of molecular biology, for example, using a PCR technique, or using a probe, or by phenotypic screening, for example, relying on a visible and traceable marker.

In a further aspect, the nucleic acid molecule according to the second aspect, or a silencing construct as defined in the first aspect, or the modification of a native regulatory sequence, may be used, alone or in combination, for the generation of a plant cell, tissue, organ, whole plant, or plant material having pathogen resistance, or for conferring or increasing pathogen resistance of in a plant, plant cell, tissue, organ, whole plant, or plant material.

In yet another aspect, a method of increasing pathogen resistance in, or a method of conferring pathogen resistance to a plant, a plant cell, tissue, organ or material may be provided, wherein the method may comprise modulation of a nucleotide sequence encoding an endogenous C-terminal domain phosphatase-like 3 (CPL3) protein or a regulatory sequence thereof, or modulation of the transcription of an endogenous CPL3 protein, wherein modulation is achieved by (i) one or more mutation(s) of the nucleotide sequence encoding a CPL3 protein, preferably wherein the one or more mutation(s) has/have a dominant negative effect, preferably wherein the one or more mutation(s) cause(s) an alteration of the amino acid sequence of the conserved catalytic domain of the CPL3 protein comprising the DXDXT/V motif; and/or (ii) one or more silencing construct(s) directed to one or more endogenous nucleotide sequence(s) encoding a CPL3 protein, preferably directed to all endogenous nucleotide sequences encoding a CPL3 protein; and/or (iii) a modification of the native regulatory sequence(s) of one or more nucleotide sequence(s) encoding an endogenous CPL3 protein, preferably all nucleotide sequences encoding an endogenous CPL3 protein, wherein the modification causes a reduced expression rate of the one or more nucleotide sequence(s) encoding an endogenous CPL3 protein may be provided. The above aspect thus covers three different modes (i) to (iii) for a targeted modulation, which may be used alone or in combination to obtain a pathogen resistant plant. According to this aspect, any one of the alternative modes of modulation according to (i) to (iii) can be used alone or in combination to achieve increased pathogen resistance, or to achieve pathogen resistance in a non-resistant plant.

In certain embodiments, it may be suitable to combine different transient and/or stable modes of modulation to obtain a maximum increase in pathogen resistance not negatively influencing the normal plant growth and development, which may depend on the total number of CPL3 alleles present in a given plant or plant cell of interest.

In yet a further aspect, the findings of the above aspects and embodiments can be favorably used for a method to identify a pathogen resistant plant, plant cell, tissue organ or material. In one embodiment, specific mutations or modifications according to the present disclosure can be used to generate a mutant or modified population of a plant, plant cell, tissue organ or material, or to identify further mutations in a relevant CPL3 gene or a regulatory sequence thereof based on the CPL3 target sequence disclosed herein and its implication for pathogen resistance in a variety of major crop plants. Particularly, mutations in a sequence homologous to the CPL3 genes/alleles or regulatory sequences as disclosed and identified herein can be identified based on the knowledge of relevant mutations and their implications for pathogen resistance in a plant. For example, comparable consensus sequences in CPL3 homologous genes can be identified to identify and thus provide further candidates involved in the modulation, preferably the increase, of pathogen resistance in a plant genome, preferably the genome of a major crop plant.

According to the various aspects and embodiment of the present disclosure, the part of the plant or plant material, or a plant cell to be mutated or modified, may be selected and optionally isolated from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

According to the various aspects and embodiments disclosed herein, the plant may be, or may originate from, a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distach-yon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yama-shitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

The various constructs for modulation of a nucleotide sequence encoding an endogenous C-terminal domain phosphatase-like 3 (CPL3) protein or a regulatory sequence thereof, or by modulation of the transcription of an endogenous CPL3 protein, or for modulating any combination of more than one CPL3 gene or allele, or the regulatory sequence or the transcript thereof, may be introduced into a plant or plant cell, tissue, organ or material by any biological, chemical or physical means. Methods of introducing biomolecules into a plant or plant cell, tissue, organ or plant material are well known in the art.

In one embodiment, a biological vector system in the context of VIGS for Agrobacterium-based transformation may include, but is not limited to, e.g., Maize Streak Virus (MSV), Barley Stripe Mosaic Virus (BSMV), Brome Mosaic virus (BMV; accession numbers: RNA 1: X58456; RNA2: X58457; RNA3: X58458), Maize Stripe Virus (MSpV), Maize Rayado Fino virus (MYDV), Maize Yellow Dwarf Virus (MYDV), Maize Dwarf Mosaic Virus (MDMV) as further detailed below under Example 1.

Further vector systems suitable for the present disclosure can generally be selected from positive strand RNA viruses of the family Benyviridae, e.g., Beet necrotic yellow vein virus (accession numbers: RNA 1: NC_003514; RNA2: NC_003515; RNA3: NC_003516; RNA4: NC_003517) or of the family Bromoviridae, e.g., viruses of the genus Alfalfa mosaic virus (accession numbers: RNA1: NC_001495; RNA2: NC_002024; RNA3: NC_002025) or of the genus Bromovirus, e.g., BMV (supra), or of the genus Cucumovirus, e.g., Cucumber mosaicvirus (accession numbers: RNA1: NC_002034; RNA2: NC_002035; RNA3: NC_001440), or of the genus Oleavirus, dsDNA viruses of the family Caulimoviridae, particularly of the family Badnavirus or Caulimovirus, e.g., different Banana streak viruses (e.g., accession numbers: NC_007002, NC_015507, NC_006955 or NC_003381) or Cauliflower mosaic virus (accession number: NC_001497), or viruses of the genus Cavemovirus, Petuvirus, Rosadnavirus, Solendovirus, Soymovirus or Tungrovirus, positive strand RNA viruses of the family Closteroviridae, e.g., of the genus Ampelovirus, Crinivirus, e.g., Lettuce infectious yellows virus (accession numbers: RNA 1: NC_003617; RNA2: NC_003618) or Tomato chlorosis virus (accession numbers: RNA 1: NC_007340; RNA2: NC_007341), Closterovirus, e.g., Beet yellows virus (accession number: NC_001598), or Velarivirus, single-stranded DNA (+/−) viruses of the family Geminiviridae, e.g., viruses of the family Becurtovirus, Begomovirus, e.g., Bean golden yellow mosaic virus, Tobacco curly shoot virus, Tobacco mottle leaf curl virus, Tomato chlorotic mottle virus, Tomato dwarf leaf virus, Tomato golden mosaic virus, Tomato leaf curl virus, Tomato mottle virus, or Tomato yellow spot virus, or Geminiviridae of the genus Curtovirus, e.g., Beet curly top virus, or Geminiviridae of the genus Topocuvirus, Turncurtvirusor Mastrevirus, e.g., Maize streak virus (supra), Tobacco yellow dwarf virus, Wheat dwarf virus, positive strand RNA viruses of the family Luteoviridae, e.g., of the genus Luteovirus, e.g., Barley yellow dwarf virus-PAV (accession number: NC_004750), or of the genus Polerovirus, e.g., Potato leafroll virus (accession number: NC_001747), single-stranded DNA viruses of the family Nanoviridae, comprising the genus Nanovirus or Babuvirus, double-stranded RNA viruses of the family Partiviridae, comprising inter alia the families Alphapartitivirus, Betapartitivirus or Deltapartitivirus, viroids of the family Pospiviroidae, positive strand RNA viruses of the family Potyviridae, e.g., comprising the genus Brambyvirus, Bymovirus, Ipomovirus, Macluravirus, Poacevirus, e.g., Triticum mosaic virus (accession number: NC_012799), or Potyviridae of the genus Potyvirus, e.g., Beet mosaic virus (accession number: NC_005304), Maize dwarf mosaic virus (accession number: NC_003377), Potato virus Y (accession number: NC_001616), or Zea mosaic virus (accession number: NC_018833), or Potyviridae of the genus Tritimovirus, e.g., Brome streak mosaic virus (accession number: NC_003501) or Wheat streak mosaic virus (accession number: NC_001886), single-stranded RNA viruses of the family Pseudoviridae, e.g., of the genus Pseudovirus, or Sirevirus, double-stranded RNA viruses of the family Reoviridae, e.g., Rice dwarf virus (accession numbers: RNA1: NC_003773; RNA2: NC_003774; RNA3: NC_003772;RNA4: NC_003761; RNAS: NC_003762; RNA6: NC_003763; RNA7: NC_003760; RNAB: NC_003764; RNA9: NC_003765; RNA10: NC_003766; RNA11: NC_003767; RNA 12:NC_003768), positive strand RNA viruses of the family Tombusviridae, e.g., comprising the genus Alphanecrovirus, Aureusvirus, Betanecrovirus, Carmovirus, Dianthovirus, Gallantivirus, Macanavirus, Machlomovirus, Panicovirus, Tombusvirus, Umbravirus oder Zeavirus, e.g., Maize necrotic streak virus (accession number: NC_007729), or positive strand RNA viruses of the family Virgaviridae, e.g., viruses of the genus Furovirus, Hordeivirus, e.g., Barley stripe mosaic virus (accession numbers: RNA1: NC_003469; RNA2: NC_003481; RNA3:NC_003478), or of the genus Pecluvirus, Pomovirus, Tobamovirus or Tobravirus, e.g., Tobacco rattle virus (accession numbers: RNA1: NC_003805; RNA2: NC_003811), as well as negative strand RNA viruses of the order Mononegavirales, particularly of the family Rhabdoviridae, e.g., Barley yellow striate mosaic virus (accession number: KM213865) or Lettuce necrotic yellows virus (accession number/specimen: NC_007642/AJ867584), positive strand RNA viruses of the order Picornavirales, particularly of the family Secoviridae, e.g., of the genus Comovirus, Fabavirus, Nepovirus, Cheravirus, Sadwavirus, Sequivirus, Torradovirus, or Waikavirus, positive strand RNA viruses of the order Tymovirales, particularly of the family Alphaflexiviridae, e.g., viruses of the genus Allexivirus, Lolavirus, Mandarivirus, or Potexvirus, Tymovirales, particularly of the family Betaflexiviridae, e.g., viruses of the genus Capillovirus, Carlavirus, Citrivirus, Foveavirus, Tepovirus, or Vitivirus, positive strand RNA viruses of the order Tymovirales, particularly of the family Tymoviridae, e.g., viruses of the order Maculavirus, Marafivirus, or Tymovirus.

In another embodiment, a physical introduction means, e.g., particle bombardment, may be chosen. In yet another embodiment, a chemical introduction means, e.g., a transfection agent, can be used. Any combination of biological, physical and chemical introduction means may be used depending on the bio-molecule(s) or constructs to be introduced, and depending on the plant cell or plant to be modified. In particular, the stability of the bio-molecule to be introduced (e.g., RNA) as well as the compartment to be targeted, or the effect to be achieved (e.g., a systemic spread to be achieved, for example, by a viral vector) should be taken into consideration.

In yet a further embodiment, the methods of the present disclosure, alone or in combination, can thus be used to engineer or select plant cells, tissues, organs, materials or whole plants with enhanced pathogen resistance in particular in maize, sorghum, wheat, sugar beet, soybean and potato plants. The technical application of the present teaching elucidating the role of the central plant immunity player CPL3 is not restricted to fungal diseases but might also be used to develop insect, bacterial, nematode and/or viral resistance in major crop plants due to the fact that the signaling pathways leading to pathogen resistance as disclosed herein are also relevant for a variety of plant pathogens, in particular also including pathogenic or parasitic fungi.

In a further aspect there is provided a method for identifying a plant having pathogen resistance or a plant cell, tissue, organ, seed, or plant material thereof, comprising the steps of: (i) isolating DNA from at least one cell of the plant or of tissue, organ, seed, or plant material thereof, and (ii) detecting at least one nucleic acid molecule as defined in the second aspect above, and optionally (iii) selecting a plant comprising at least one nucleic acid molecule as defined in the second aspect above based on the detection in step (ii), and optionally (iv) breeding progeny having pathogen resistance through crossing of the plant selected in step (iii) with another plant, preferably of the same species, and thereby introducing the at least one nucleic acid molecule detecting in step (ii) in to the genome of the progeny.

The present invention will now be illustrated by reference to the following Examples, which are not construed to limit the scope of the present invention.

### EXAMPLES

### Example 1A: CPL3 Downregulation in Wheat Results in Increased Resistance Against the Hemibiotrophic Fungal Pathogen Zymoseptoria Tritici

Two silencing constructs targeting all three homologues of TaCPL3 for virus induced gene silencing (VIGS) experiments in wheat (SEQ ID NO: 21 and 22) were specifically developed. The two silencing constructs TaCPL3_fragA and TaCPL3_fragB were specifically designed for having high homology (>95% identity) to all three TaCPL3 homologues at the same time. At the same time, specific efforts were made to avoid large stretches of homology to other coding regions in the wheat genome to avoid undesired off-target effects. Preferably, no more than 20 bp of contiguous identity should be present to another region in the genome, more preferably as few identities as possible to any off-target region should be present.

For the VIGS experiments the protocol described in Yuan et al. (2011, Plos One, 6(10), e26468) was used. Suitable vector systems for Agrobacterium based transformation suitable for the purpose of the present invention are well known in the art and include, but are not limited to, e.g., MSV, BSMV, BMV, MSpV, MYDV, MYDV, or MDMV.

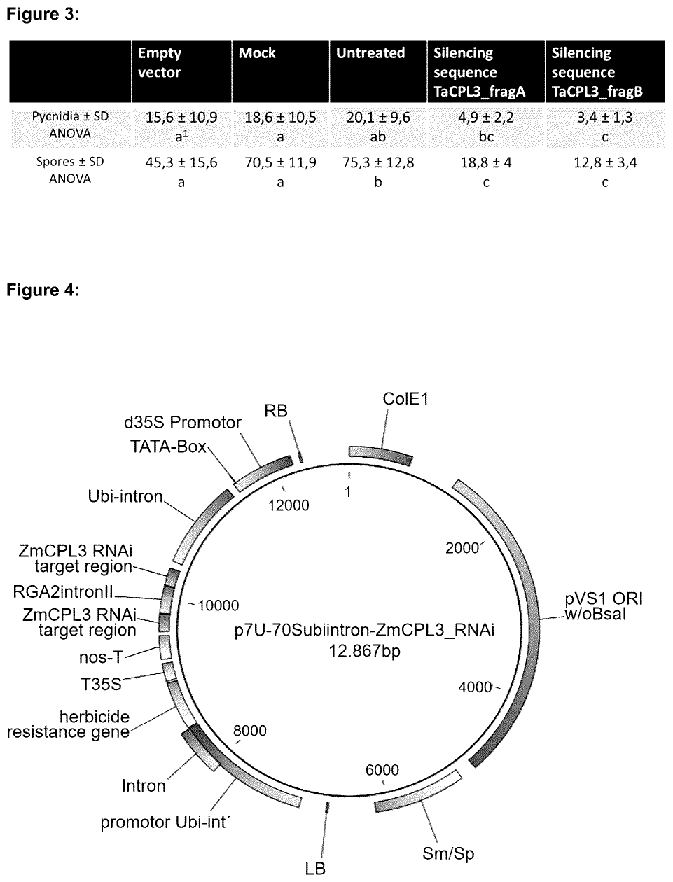

After transformation of Nicotiana benthamiana with the viral vectors encoding for the silencing constructs, leaves of wheat cultivar Taifun were subsequently transfected with sap extracted from the transformed Nicotiana benthamiana leaves. 14 days after transfection with the different viral constructs encoding the targeting sequences against TaCPL3-A (SEQ ID NO: 5), TaCPL3-B (SEQ ID NO: 6) and TaCPL3-D (SEQ ID NO: 7), the wheat plants were infected with Zymoseptoria tritici spore suspension (Millyard et al., 2016. The ubiquitin conjugating enzyme, TaU4 regulates wheat defence against the phytopathogen Zymoseptoria tritici. Scientific reports, 6, 35683.). The plants were kept under plastic hoods for 4 days to increase the humidity for optimal Septoria infection conditions. 23 days after infection, 2 infected leaves per plant were detached and incubated on agar plates. Under these conditions of high humidity pycnidia form on the leaves that were counted over an area of 2 cm per leaf 10 days after transfer to the agar plates. In addition, spores from five leaves were washed off with 10 ml of water and spores were counted using a hemocytometer. In comparison to wheat plants that were mock-inoculated, untreated or infected with an empty vector control, the wheat plants infected with CPL3-silencing constructs showed a significant reduction of pycnidia and spore count (FIG. 3).

The data demonstrate that silencing of all three CPL3 homologues in wheat leads to increased resistance against the hemibiotrophic fungal pathogen Zymoseptoria tritici.

### Example 1B: CPL3 Downregulation in Wheat Results in Increased Resistance Against the Fungal Pathogen Fusarium Graminearum

Two silencing constructs targeting all three homologues of TaCPL3 for virus induced gene silencing (VIGS) experiments in wheat (SEQ ID NO: 21 and 22) were tested to increase the Fusarium graminearum resistance of wheat. The VIGS inoculation experiments with the two silencing constructs TaCPL3_fragA and TaCPL3_fragB were done as described for example 1A.

After the onset of wheat heads two spikelets in the middle of each head were inoculated with 25 μl/25.000 spores of Fusarium graminearum. 10, 14 and 21 days after inoculation the bleaching of the heads by F. graminearum was measured (Table 8; FIG. 7).

TABLE 8
VIGS mediated gene silencing of CPL3A and CPL3B by BSMV resulted in reduced
head scab symptoms of Fusarium graminearum infected wheat heads of the cultivar Taifun.

| Fusarium head | Empty | Silencing | Untreated | CPL3A | CPL3B |
|---|---|---|---|---|---|
| scab symptoms | vector (%) | control (%) | (%) | (%) | (%) |
| 10 dpi | | | | | |
| 14 dpi | 30.6 ± 15.9 | 41 ± 21.4 | 52 ± 27.7 | 20 ± 4.1 | 20.6 ± 5.8 |
| 21 dpi | 71.3 ± 25.9 | 75 ± 25.6 | 93 ± 15.7 | 23.8 ± 4.8 | 47.8 ± 23.6 |

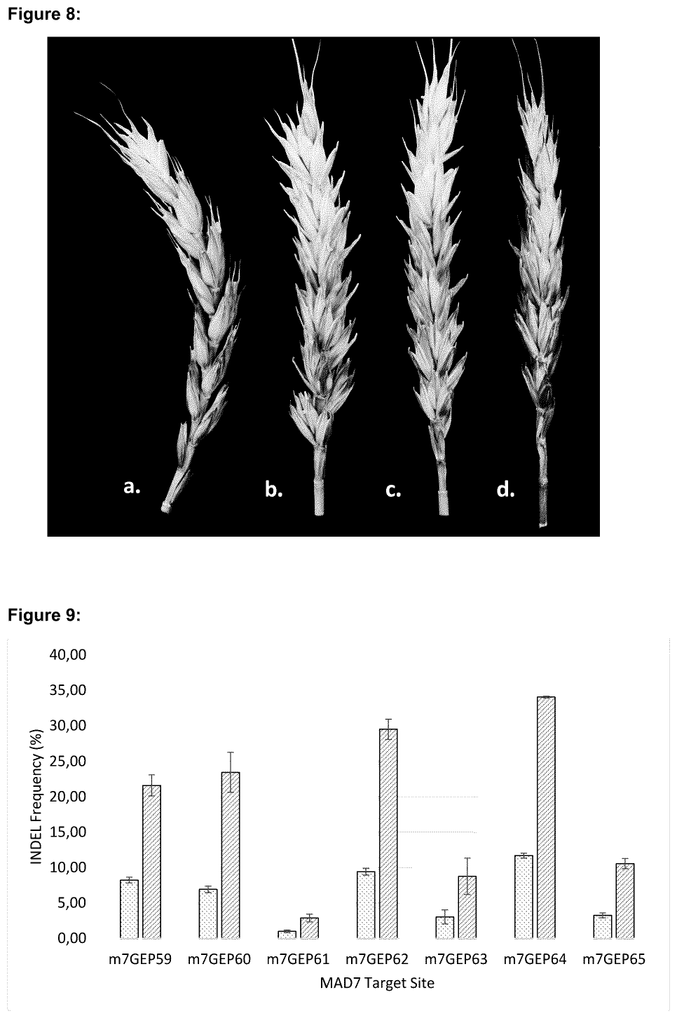

In comparison to wheat plants that were infected with the empty vector and a silencing control or were not virus-infected (untreated), the wheat plants infected with CPL3-silencing constructs showed a strong reduction of symptoms (FIG. 8).

The data demonstrate that silencing of all three CPL3 homologues in wheat enhances resistance the Fusarium head scab.

### Example 2: CPL3 Downregulation in Corn Results in Increased Resistance Against Setosphaeria Turcica

For testing the effect of CPL3 downregulation on pathogen resistance in the relevant crop plant Zea mays, an RNAi silencing construct against ZmCPL3 was developed (FIG. 4 and SEQ ID NO: 24) and stably transformed maize plants of the A188 genotype. The ZmCPL3 silencing sequence (SEQ ID NO: 23) was specifically selected for having perfect homology to the ZmCPL3 gene and, at the same time to avoid large stretches of homology (<20 bp) to other sequences in the maize genome. Transgenic maize plants of the segregating T1 generation as well as the homozygous T2 lines along with the respective azygous sister lines (null-segregants) were tested for resistance against Setosphaeria turcica (Northern Corn Leaf Blight) in the greenhouse.

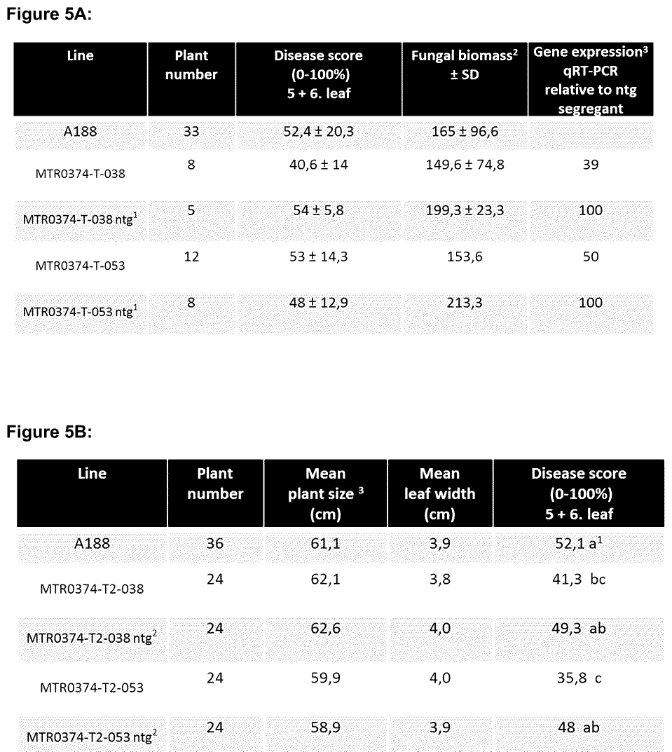

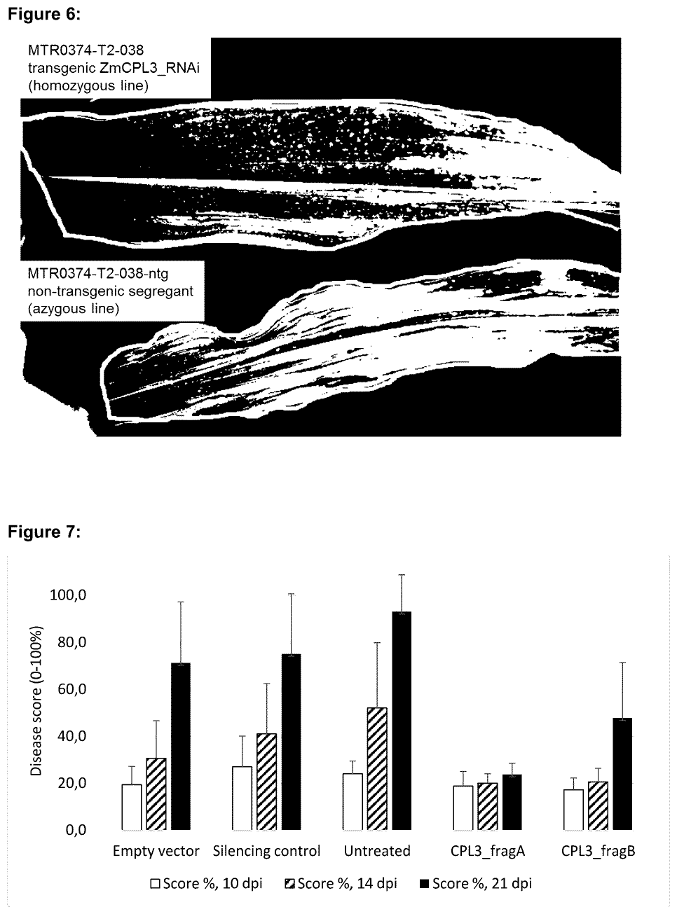

The experiments showed that two independent transgenic lines expressing the ZmCPL3-silencing construct were more resistant to NCLB than the respective null-segregants or the transformation genotype A188 (FIGS. 5A-5B and 6). In the same experiments, plant height and width of the fully emerged forth leaf shortly before infection to determine if ZmCPL3 downregulation affects plant growth was measured. The results showed that there was no difference in plant growth between the ZmCPL3 RNAi transgenic plants and the respective null-segregants (FIGS. 5A-5B). This finding was not necessarily expected as influencing the central plant immune effector CPL3 (in a knock-out) was reported to be associated with decreased growth and development. The inventors thus also analyzed the expression of ZmCPL3 in the transgenic line by qRT-PCR and were able to confirm that ZmCPL3 expression was reduced by the silencing constructs specifically developed.

These results confirm that downregulation of ZmCPL3 leads to increased resistance against a hemibiotrophic fungus and that downregulation of ZmCPL3 does not cause growth retardation.

### Example 2B: Targeted Knock-Out of the Maize CPL3 Gene

The Zm-CPL3 sequence, A188v1_046614, was used for gene knock-out by CRISPR genome editing. In this case it has been looked for an active target site in the predicted Zm-CPL3 open reading frame (ORF) that would generate a targeted double-stranded break in the DNA. The desired outcome was DNA repair at the cut site by the NHEJ pathway leading to random deletion and/or insertions (INDELs) that could interrupt the normal coding sequence of the Zm-CLP3 gene. Target site activity was assayed initially by using amplicon deep sequencing and next generation sequencing (NGS—Illumina sequencing) to measure the DNA cutting frequency in maize protoplasts. The NGS data was used to identify and then select an individual target site with adequate activity for use in a maize tissue culture and transformation system for recovery of plants. Maize plants were generated after transformation with the selected target site and CRISPR constructs that demonstrated a variety of INDELs in the Zm-CPL3 gene and are likely to knock-out gene function by interruption of the coding sequence.

The Cas12a CRISPR nucleases, named Cpf1 following their initial discovery, have now been derived from a number of source organisms. In the work for the knock-out of ZmCPL3 we used a related nuclease of the Type V (CPF1-like) Cas family. The nuclease is called MAD7 due to its initial discovery in microbes from Madagascar and was obtained from INSCRIPTA™. The gene was modified for optimal maize codons in order to enhance transcription and expression of the nuclease in transformed maize tissue. Constructs were built that express the MAD7 constitutively from the Bd-Ubi promoter that could be used for both the initial protoplast characterization work as well as corn transformation experiments.

A188 protoplasts were isolated, divided into cells for transfection, and separately transfected using constructs pGEZM008-pGEZM011 that carried expression cassettes designed to constitutively express the CRISPR RNA's (crRNA) m7GEP59-m7GEP65 (Table 9). A separate construct, pGEP837, with the maize optimized MAD7 gene linked to the Bd-Ubi promoter and double 35S promoter driven green fluorescent protein gene was co-transfected with each of the crRNA constructs. In this way, each protoplast sample had the combination of constructs for constitutive expression of MAD7 and a unique crRNA with cutting activity targeted to independent sites in the Zm-CPL3 gene. The fluorescent protein was used to determine the transfection efficiency by counting fluorescent cells using a flow cytometer. FIG. 9 shows data from these experiments and each bar is the result of samples that were replicated 3 times using independent co-transfections. Two target sites stood out as having the highest activity that were targeted by crRNA's m7GEP62 and m7GEP64 and expressed by constructs pGEZM008 and pGEZM010 respectively. Based on the position in the Zm-CPL3 gene, m7GEP62 (pGEZM008) was choosen for advancement into plant transformation work.

TABLE 9
List of Zm-CPL3 crRNA sequences and their corresponding constructs
used for both protoplast transfection and plant transformation.
CRISPR nuclease activity, MAD7 included, requires a protospacer
adjacent motif (PAM) sequence in addition to the protospacer
sequence that directs where in the Zm-CPL3 that the double stranded
break occurs.

| crRNA | PAM | Protospacer | SEQ ID | |
|---|---|---|---|---|
| Name | Sequence | Sequence (Target) | NO: | Construct |
| m7GEP59 | TTTC | CTCGTCCTTGGGCGTGACCGT | 25 | pGEZM005 |
| m7GEP60 | TTTG | GTCACTGCTGCCGGGGGCGGG | 26 | pGEZM006 |
| m7GEP61 | TTTC | GCTATGCCTTCAATAGCTTTG | 27 | pGEZM007 |
| m7GEP62 | TTTG | CGTGGTCGCAGGCCGTGCGGA | 28 | pGEZM008 |
| m7GEP63 | TTTG | GACTCCGACGCCCCGGAGAAG | 29 | pGEZM009 |
| m7GEP64 | TTTC | AGGTGTCTGAGAAAACCAGTT | 30 | pGEZM010 |
| m7GEP65 | TTTG | TCAGACACCTGAAACAAAGCC | 31 | pGEZM011 |

Maize (A188) transformation for genome editing was done by using a rapid regeneration protocol based on the RBP2 gene (WO 2019/238909) that promotes de-novo embryogenesis from differentiated recipient cells in immature maize embryos. Particle bombardment was used to introduce pGEMT129, pGEZM008, and pGEMT128 into recipient cells. The construct pGEMT129 has the same constitutive MAD7 gene as pGEP837, but includes the tdTomato gene which is useful in indicating how efficiently the DNA was delivered to embryos by particle bombardment. The RBP2 expression cassette with Bd-EF1 promoter is included in pGEMT128. Plates of maize immature embryos (50 ct) are bombarded 3 times with 0.6 μM gold particles (BioRad) coated by these plasmids and associated using the CaCl₂+spermidine protocol. Bombardments were done using the 450 PSI rupture discs in order to try to minimize cell damage. Plants were regenerated using a series of tissue culture medium changes and finally recovered in plastic containers as small, rooted corn plants. The young corn plants were sampled for molecular analysis at this stage.

TABLE 10
Transformation and genome editing frequencies of maize A188 immature embryos.
Maize plants were generated from immature embryos following pGEP1054 +
pGEZM008 particle bombardment. Plant leaf samples were taken and used for DNA
extraction followed by PCR amplification around the target sequence. Amplicons were
Sanger sequenced to identify the presence of INDEL at the m7GEP62 target site
and the results of plants still in medium are indicated in the column labelled Assay 1.
Plants were then transplanted to soil and recovered to the greenhouse.
Surviving plants were assayed again (Assay 2) for the presence of INDEL.

| Experiment | Imm. Embryo (ct.) | Regeneration (ct.) | Assay 1 (ct.) | Assay 2 (ct.) |
|---|---|---|---|---|
| GEZM054-5 | 150 | 211 (141%) | 7 (3.3%) | 3 (1.4%) |
| GEZM054-6 | 150 | 89 (59%) | 3 (3.3%) | 2 (2.2%) |
| GEZM054-7 | 150 | 460 (307%) | 20 (6.5%) | 10 (2.1%) |
| GEZM054-8 | 150 | 159 (106%) | 7 (4.4%) | 5 (3.1%) |

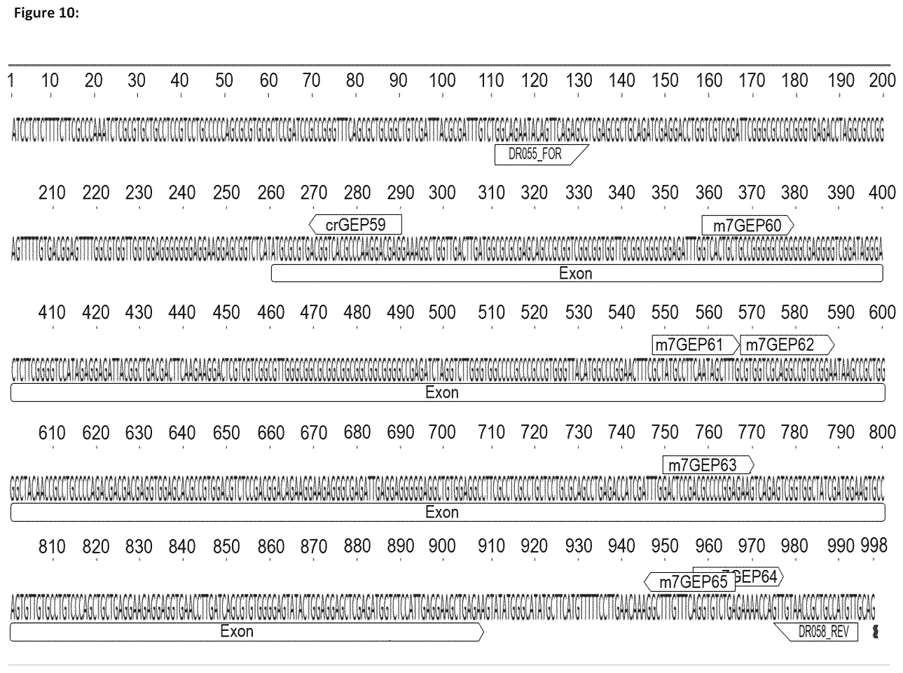

Maize shoots regenerated from bombarded immature embryos were recovered into Phytatrays™ (Sigma) on medium to promote their growth and development. Containers were maintained in the Conviron growth room under long day lighting regimes and at constant temperature and humidity. Transformed maize tissue usually regenerated 1 plant per event but at a low to moderate frequency multiple plants per event were regenerated. In the case of multiple shoots per event, all of the leaf tips were sampled and pooled as one for DNA extraction. Extracted DNA was used for PCR amplification of the sequence flanking the target site (primers, FIG. 10). A portion of the PCR reactions were run on a gel to visually confirm that the amplicon was present and running at the expected size. The remaining portion of the PCR reaction was cleaned up for primer and reagents using ExoSAP-It reagent and submitted directly for Sanger sequencing. Sequence alignments to an unmodified reference sequence were used to identify the candidates with INDEL because those would demonstrate mixed trace results originating from the type of INDEL and whether it was present on one or both alleles of the Zm-CPL3 gene. A mixed trace is the region in the sequence trace file where multiple base calls are present in each position making the base calls ambiguous. Table 10 shows the results of the process from 4 independent experiments. In each of these experiments there were 150 immature embryos bombarded and initiated in tissue culture. Regeneration of maize plants varied between 59% and 307% with the latter regenerating multiple plants per initiated embryo. The Assay 1 column shows results of the 4 experiments and the frequency of events with detected INDEL that were available to be advanced to the greenhouse. The column Assay 2 contains the final numbers at the TO stage and includes the plants that survived the transfer to the greenhouse and then passed a second screen by PCR and Sanger sequencing. These will be the T0 plants most likely to yield T1 progeny with the Zm-CPL3 edits.

Sanger sequence trace files (ABI files) provide an indication of which plants to select for advancement to the greenhouse but they do not offer the resolution to provide the precise sequence at one or both Zm-CPL3 alleles in the DNA sample of the plant genome. There are a variety of methods that could be employed to demonstrate the sequence of each allele including analysis of the T1 progeny following genetic segregation of the alleles. Finally, a variety of new edits has been created (Table 11). Most of the edits were deletions as is typical for MAD7 and Cpf1 nucleases. Many of the edits shown disrupt the open reading frame of the CPL3 gene and thus the gene function will be eliminated or knocked out.

TABLE 11
Amplicon sequencing of selected maize A188 events targeting the CPL3 gene. DNA
extracted from T0 plants were PCR amplified for the targeted sites in exon 1 of
Zm-CPL3. Analysis resolves the INDEL's represented by the mixed trace and showed
a wide variety of edits produced by this genome targeting approach. A subset of
the total events generated is shown in the table.

| | | | SEQ ID |
|---|---|---|---|
| Experiment | T0 Event Name | Sequence | NO: |
| na | (A188 reference) | TAGCCCCAGCGGCTTAT\|TCCGCA\|CGGCCTGCGACCACGCAAA | 32 |
| GEZM054-5 | GEZM054-T811 | TAGCCCCAGCGGCTT--\|------\|CGGCCTGCGACCACGCAAA | 33 |
| GEZM054-5 | GEZM054-T821 | TAGCCCCAGCGGCTTAT\|TCC---\|-GGCCTGCGACCACGCAAA | 34 |
| GEZM054-5 | GEZM054-T868 | TAGCCCCAGCGG-----\|------\|-----TGCGACCACGCAAA | 35 |
| | | TAGCCCCAGCGGCTTAT\|TC--CA\|CGGCCTGCGACCACGCAAA | 36 |
| GEZM054-6 | GEZM054-T929 | TAGCCCCAGCGGCTT--\|------\|--GCCTGCGACCACGCAAA | 37 |
| | | TAGCCCCAGCGGCTT--\|------\|CGGCCTGCGACCACGCAAA | 38 |
| GEZM054-6 | GEZM054-T941 | TAGCCCCAG--------\|------\|CGGCCTGCGACCACGCAAA | 39 |
| GEZM054-7 | GEZM054-T1207 | TAGCCCCAGCGGC----\|------\|-GGCCTGCGACCACGCAAA | 40 |
| | | TAGCCCCAGCGGCTTAT\|TC----\|CGGCCTGCGACCACGCAAA | 41 |
| GEZM054-7 | GEZM054-T1260 | TAGCCC-----------\|------\|CGGCCTGCGACCACGCAAA | 42 |
| | | TAGCCCCAGCGGCTTAT\|TC----\|CGGCCTGCGACCACGCAAA | 43 |
| GEZM054-7 | GEZM054-T1305 | TAGCCCCAGCGGCTTAT\|TCC---\|--GCCTGCGACCACGCAAA | 44 |
| GEZM054-8 | GEZM054-T592 | TAGCCCCAGCGGC----\|------\|-GGCCTGCGACCACGCAAA | 45 |
| GEZM054-8 | GEZM054-T602 | TAGCCCCAGCGGCTT--\|------\|--GCCTGCGACCACGCAAA | 46 |
| GEZM054-8 | GEZM054-T626 | TAGCCCCAGCGGCTTA-\|------\|----------CCACGCAAA | 47 |

### Example 3: CPL3 Downregulation in Dicots, namely Beta Vulgaris

To confirm the relevance of the CPL3 gene in sugar beet (Beta vulgaris) (SEQ ID NO: 4 and 14) for fungal resistance the inventors intend to search for (knock-out) mutations in this gene by a TILLING approach using EMS or ENU as mutagen. The selected plants will subsequently self-pollinated to create homozygous mutants. The homozygous CPL3 mutants will be analyzed for fungal resistance, for example against Cercospora beticola. The resistance assay with sugar beet and Cercospora beticola could be performed as described by Schmidt et al. (Plant Mol Biol, (2004). Suppression of phenylalanine ammonia lyase expression in sugar beet by the fungal pathogen Cercospora beticola is mediated at the core promoter of the gene. Plant molecular biology, 55(6), 835-852.). Without wishing to be bound by theory, it is expected that CPL3 knock-out sugar beet plants will show increased resistance. To test potential side-effects as growth retardation, it is intended to rely on full knock-outs of the respective genes, or a strategy relying on a transient modulation by a silencing construct, or a further strategy relying on the creation and/or provision of a dominant negative CPL3 allele to test the different outcomes on resistance in a targeted way.

### Example 4: CPL3 Downregulation—Effect in Pathogen Resistance

Given the central role of CPL3 identified herein, the potential of CPL3 modulation in different target plants was addressed. First, relevant target crop plants of economic and agronomic interest were defined. As a next step, the most severe pathogens from all taxa, in part very specific for certain target plants, were defined. To test whether CPL3 modulation can be advantageous for enhanced pathogen resistance against a variety of pathogens, including viral, bacterial, oomycete, nematode, insect or fungal pathogens, target pathogen types and the correlated diseases will thus be studied in various plant models to define the extent of CPL3 modulation needed and the specific way of CPL3 modulation needed (i.e., on RNA level and/or DNA level or even protein level) to achieve enhanced pathogen resistance by biological rather than chemical means for a several plant pathogens in the respective target plant causing major losses in harvest and crop production.

1-15. (canceled)

16. A wheat plant having pathogen resistance, wherein pathogen resistance is conferred or increased by downregulation of a nucleotide sequence encoding an endogenous C-terminal domain phosphatase-like 3 (CPL3) protein or a regulatory sequence thereof, or by modulation of the transcription of an endogenous CPL3 protein, wherein modulation is achieved by
(i) one or more mutation(s) of the nucleotide sequence encoding a CPL3 protein, wherein the one or more mutation(s) has/have a dominant negative effect, preferably wherein the one or more mutation(s) cause(s) an alteration of the amino acid sequence of the conserved catalytic domain of the CPL3 protein comprising the DXDXT/V motif; and/or
(ii) one or more silencing construct(s) directed to one or more endogenous nucleotide sequence(s) encoding a CPL3 protein, preferably directed to all endogenous nucleotide sequences encoding a CPL3 protein; and/or
(iii) a modification of the native regulatory sequence(s) of one or more nucleotide sequence(s) encoding an endogenous CPL3 protein, preferably of all native regulatory sequence(s) of the nucleotide sequences encoding an endogenous CPL3 protein, wherein the modification causes a reduced expression rate of the one or more nucleotide sequence(s) encoding an endogenous CPL3 protein,
wherein the nucleotide sequence encoding the CPL3 protein is selected from the group consisting of:
(a) a nucleotide sequence set forth in SEQ ID NO: 5 or a homologous, orthologous or paralogous sequence thereof;
(b) a nucleotide sequence having at least 80% identity to the one of nucleotide sequences as defined in (a);
(c) a nucleotide sequence encoding for the amino acid sequence set forth in SEQ ID NO: 15 or for an amino acid sequence which have at least 80% identity to the one sequence as set forth in SEQ ID NO: 15;
(d) a nucleotide sequence encoding for an amino acid sequence having at least 80% identity to one of the sequences set forth in SEQ ID NO: 15, or
(d) a nucleotide sequence hybridizing with a nucleotide sequence complementary to the sequence as defined in (a)-(d) under stringent conditions.

17. The plant according to claim 16, wherein the pathogen is at least one of a fungal pathogen, an oomycete pathogen, a bacterial pathogen, a virus, a nematode pathogen, or an insect, preferably wherein the pathogen is a hemibiotrophic fungus selected from the group consisting of: Zymoseptoria tritici, Setosphaeria turcica, Fusarium spp. Fusarium graminearum, Colletotrichum spp. such as Colletotrichum graminicola, Magnaporthe grisea, Magnaporthe oryzae, Phytophthora infestans, or wherein the pathogen is a fungus selected from Cercospora spp., preferably Cercospora beticola or Cercospora zeae-mayidis.

18. A wheat plant according to claim 16, wherein the one or more mutation(s) has/have a dominant negative effect and is/are present in the heterozygous state in the plant.

19. A wheat plant according to claim 16, wherein the one or more mutation(s) is/are (a) mutation(s) of the nucleotide sequence encoding a CPL3 protein causes the substitution of Asp by Ala at position 938 referenced to SEQ ID NO: 15.

20. The wheat plant according to claim 16, wherein the one or more silencing construct(s) comprise(s):
I. an RNAi molecule directed against, targeting, or hybridizing with the nucleotide sequence encoding the CPL3 protein, or a polynucleotide sequence encoding said RNAi molecule; or
II. an RNA-specific CRISPR/Cas system directed against or targeting the nucleotide sequence encoding the CPL3 protein, or a polynucleotide sequence encoding said RNA-specific CRISPR/Cas system, preferably wherein the RNAi molecule is selected from a dsRNA molecule, a shRNA molecule, a miRNA molecule or a siRNA molecule which comprises at least 15, 16, 17, 18, 19, 20, 21, 22, 23, 24, 25, 26, 27, 28, 29, 30, 31, 32, 33, 34, 35, 36, 37, 38, 39, 40, 45 or 50 contiguous nucleotides of the coding nucleotide sequence of the CPL3 protein or the complementary sequence thereof in sense or antisense direction, more preferably wherein the RNAi molecule is selected from a sequence of SEQ ID NOs: 21 to 24, or a sequence having at least 95%, 96%, 97%, 98% or 99% sequence identity thereto.

21. The wheat plant according to claim 20, wherein the RNAi molecule does not share substantial sequence identity with other genomic regions in the genome of the wheat plant.

22. The wheat plant according to claim 16, wherein the modification of the native regulatory sequence(s) is a transient or stable modification of a regulatory sequence, preferably wherein
(i) the modification is introduced by a site-directed DNA modifying enzyme, or
(ii) a modified site-directed DNA modifying enzyme mediates the modification, preferably the inhibition, of a regulatory sequence, or
(iii) the modification is introduced by random mutagenesis, preferably wherein the random mutagenesis is selected from chemical-induced mutatgenesis or irradiation-induced mutagenesis.

23. A cell, tissue, organ, seed or material of a wheat plant according to claim 16.

24. A nucleic acid molecule comprising a nucleotide sequence encoding for a C-terminal domain phosphatase-like 3 (CPL3) protein, wherein the nucleotide sequence is selected from the group consisting of:
(a) a nucleotide sequence set forth in SEQ ID NO: 5;
(b) a nucleotide sequence having at least 80% identity to the nucleotide sequences as defined in (a),
(c) a nucleotide sequence encoding for an amino acid sequence set forth in SEQ ID NO: 15; or
(d) a nucleotide sequence hybridizing with a nucleotide sequence complementary to the sequence as defined in (a)-(c) under stringent conditions,
wherein the nucleotide sequence comprises at least one mutation capable of conferring or increasing resistance to a pathogen in plant in which the nucleic acid molecule is expressed, preferably wherein the pathogen is at least one of a fungal pathogen, an oomycete pathogen, a bacterial pathogen, a virus, a nematode pathogen, or an insect, preferably wherein the pathogen is a hemibiotrophic fungus selected from the group consisting of: Zymoseptoria tritici, Setosphaeria turcica, or wherein the pathogen is a fungus selected from Cercospora spp., preferably Cercospora beticola or Cercospora zeae-mayidis.

25. The nucleic acid molecule according to claim 24, wherein the mutation is a mutation of the nucleotide sequence encoding a CPL3 protein, having a dominant negative effect, preferably wherein the mutation causes an alteration of the amino acid sequence of the conserved catalytic domain of the CPL3 protein comprising the DXDXT/V motif, more preferably wherein the mutation is a mutation of the nucleotide sequence encoding a CPL3 protein causes the substitution of Asp by Ala at position 938 referenced to SEQ ID NO: 15.

26. A method of generating a wheat plant having pathogen resistance or a cell, tissue, organ, seed, or material thereof, comprising the steps of:
(i) providing one or more silencing construct(s) as defined in claim 16, or one or more sequences encoding the same;
(ii) modifying the cell, tissue, organ, plant, seed, or material by introducing the one or more silencing construct(s) or the sequence encoding the same of (i), into the genome of said cell, tissue, organ, plant, seed, or material; and
(iii) obtaining the modified cell, tissue, organ, plant, seed or material,
(iv) optionally, regenerating a wheat plant from the cell, tissue, organ or material or growing a seed on a wheat plant obtained in (iii),
wherein the cell, tissue, organ, plant, seed or material obtained in (iii), the plant regenerated in (iv) or the seed grown in (iv) comprises the introduced one or more silencing construct(s) or the sequence encoding the same and thereby has pathogen resistance.

27. A method of generating a wheat plant having pathogen resistance or a cell, tissue, organ, seed, or material thereof, comprising the steps of:
(i) providing at least one site-directed DNA modifying enzyme, or a sequence encoding the same, and optionally at least one DNA repair template, wherein the at least one site-directed DNA modifying enzyme and optionally the at least one DNA repair template:
(a) is/are directed or targeted to the nucleotide sequence encoding the CPL3 protein as defined in claim 16; or
(b) is/are directed or targeted to the regulatory sequence of at least one CPL3 protein encoding nucleotide sequence as defined in claim 16;
(ii) introducing the at least one site-directed DNA modifying enzyme or a sequence encoding the same, and optionally the at least one DNA repair template into the cell, tissue, organ, plant, or material;
(iii) mutating or modifying the nucleotide sequence encoding the CPL3 protein or the regulatory sequence thereof in the genome of the cell, tissue, organ, plant, or material and obtaining a mutant or modified population of cells, tissues, organs, plants, or plant materials;
(iv) optionally: screening the population for a dominant negative mutation, thereby conferring or increasing pathogen resistance, or screening the population for a mutation or modification in the nucleotide sequence encoding the CPL3 protein or the regulatory sequence thereof; and
(v) identifying and thereby obtaining a plant cell, tissue, organ, plant, or plant material having pathogen resistance.

28. A method of generating a wheat plant having pathogen resistance or a cell, tissue, organ, seed, or material thereof, comprising the steps of:
(i) subjecting the cell, tissue, organ, plant, or material, preferably seeds of a wheat plant, to an efficient amount of a mutagenic agent, preferably ethylmethane sulfonate, N-ethyl-N-nitrosourea, or radiation,
(ii) obtaining a mutagenized population of cells, tissues, organs, plants, or materials, optionally by growing wheat plants from the mutagenized population;
(iii) screening the mutagenized population for pathogen resistance, optionally by isolating and analyzing genomic DNA from the plants having pathogen resistance;
(iv) identifying and obtaining a modified cell, tissue, organ, plant, or material having pathogen resistance.
